(19) United States
(12) Patent Application Publication
(10) Pub. No.: US 2026/0055368 A1
(43) Pub. Date: Feb. 26, 2026

(54) METHODS FOR GENERATING GAMMA DELTA T-CELLS AND RELATED COMPOSITIONS

(71) Applicant: KIROMIC BIOPHARMA, INC., Houston, TX (US)

(72) Inventors: Leonardo MIRANDOLA, Plano, TX (US); Alan GUERRERO, Katy, TX (US); Joshua Nathan KELLNER, Richmond, TX (US)

(21) Appl. No.: 18/876,630

(22) PCT Filed: Jun. 20, 2023
(86) PCT No.: PCT/US2023/068753

§ 371 Date: Dec. 18, 2024

Related U.S. Application Data
(60) Provisional application No. 63/354,027, filed on Jun. 21, 2022.

(51) Int. Cl. C12N 5/0783; C07K 14/725; C07K 16/28; C07K 16/30; C12N 5/00; C12N 15/86

(52) U.S. Cl. CPC C12N 5/0636; C07K 14/7051; C07K 16/2818; C07K 16/30; C12N 5/525; C12N 5/562; C12N 15/86; C07K 2317/92; C12N 2501/2302; C12N 2501/2307; C12N 2501/999; C12N 2740/10043

(57) ABSTRACT

Provided herein, inter alia, are compositions and methods including T-cell cultures enriched for gdT cells, the gdT cells expressing a CAR, and related methods for generating said cells. In an aspect provided herein is a method for generating a T-cell culture enriched for gamma delta T-cells (gd T-cells or T cells). In another aspect, a method for generating a gdT-cell expressing a Chimeric Antigen Receptor (CAR) is provided. The method includes introducing a nuclei acid encoding a CAR to a gdT-cell obtained as provided herein, including embodiments thereof.

### CROSS-REFERENCE TO RELATED APPLICATIONS

This International Application claims the benefit of priority under 35 U.S.C. § 119(e) of U.S. Provisional Application No. 63/354,027, filed on Jun. 21, 2022, which is hereby incorporated by reference in its entirety and for all purposes.

### REFERENCE TO A SEQUENCE LISTING

The material in the accompanying Sequence Listing is hereby incorporated by reference in its entirety. The accompanying file, named “060851-512001WO_SL_ST26.xml” was created on Jun. 20, 2023 and is 233,472 bytes.

### BACKGROUND OF THE INVENTION

Methods for generating a T-cell culture enriched for gamma delta T-cells have been described, but can have other cells contaminating the culture. Consequently, there is a need for compositions and methods that result in greater proportion of gamma delta T-cells in such cultures. In addition, T-cell therapies are being used to treat a wide variety of cancers, but some cancers can be resistant to such therapies. Consequently, there is a need for compositions and methods that allow for more effective cancer therapies.

### BRIEF SUMMARY OF THE INVENTION

In an aspect provided herein is a method for generating a T-cell culture enriched for gamma delta T-cells (gd T-cells or γδ T cells). The method includes: (a) contacting a population of immune cells with a first media composition thereby forming an initial cell culture, wherein the population of immune cells includes alpha beta T-cells (abT-cells), gdT-cells, and monocytes, wherein the ratio of abT-cells to gdT-cells in the population of immune cells is at least about 16:1, wherein the ratio of monocytes to gdT-cells is at least about 2:1, and wherein the first media composition comprises a bisphosphonate, interleukin-7 (IL-7) and interleukin-2 (IL-2); (b) incubating the initial cell culture for a first time period of about one to about three days, thereby forming a monocyte-depleted immune cell culture; (c) after the first time period, contacting the monocyte-depleted cell culture with interleukin-7 (IL-7) and interleukin-2 (IL-2), but not a bisphosphonate, thereby forming a monocyte-depleted cell culture in contact with a second media composition; (d) incubating said monocyte-depleted cell culture in contact with the second media composition for a second time period of about one to about three days; thereby forming an expanded gdT cell culture, wherein the ratio of abT-cells to gdT-cells in the expanded gdT cell culture is less than about 10:1; (e) removing abT-cells from the expanded gdT cell culture thereby forming an abT-cell-depleted culture; (f) replacing said second media composition with a third media composition comprising interleukin-7 (IL-7) and interleukin-2 (IL-2), wherein the third media composition does not comprise a bisphosphonate; and, (g) incubating the abT-cell-depleted culture for at least one day in contact with the third media composition, thereby forming a T-cell culture enriched for gdT-cells, wherein the ratio of abT-cells to gdT-cells in the T-cell culture enriched for gdT-cells is less than about 1:2.

In another aspect a method for generating a gdT-cell expressing a Chimeric Antigen Receptor (CAR) is provided. The method includes introducing a nuclei acid encoding a CAR to a gdT-cell obtained as provided herein, including embodiments thereof.

In another aspect is provided a T-cell culture enriched for gdT-cells as provided herein, including embodiments thereof.

In another aspect is provided a T-cell population expressing a Chimeric Antigen Receptor (CAR), including the T-cell culture enriched for gdT-cells as provided herein, including embodiments thereof. The gdT-cells include a nucleic acid encoding the CAR.

### BRIEF DESCRIPTION OF THE DRAWINGS

FIG. 1 shows the cell viability of the gdT-cell population using the DeltaCel Process harvested at Day 8 (mean: 88.6%) vs. Day 9 (mean 89.2%) N=3.

FIG. 2 shows the total fold expansion of the gdT-cell population using the DeltaCel Process harvested at Day 8 (mean: 71.1%) vs. Day 9 (mean: 117.6%). N=3.

FIG. 3 shows cell viability of the gdT-cell population using the DeltaCel Process harvested at either Day 8 or Day 9, cryopreserved in CS10 versus CS5. n=1. Day 8: CS10, 89%; CS5, 89%. Day 9: CS10, 57%; CS5, 66%. N=1.

FIG. 4 shows cell viability of the gdT-cell population using the DeltaCel Process harvested at Day 8. Cells were cryopreserved in either CS10 or CS5 at 50e6. (50e6 cells/ml: CS10, mean=72%, CS5, mean=89%; 20e6 cells/ml: CS10, mean=72%, CS5, mean=77%). N=2.

FIG. 5 shows cell cytoxicity/potency of DeltaCel Process harvested at Day 8 and cryopreserved in either CS10 or CS5 at 50e6 cells/ml and 20e6 cells/ml. (50e6 cells/ml: CS10, mean=18.2%, CS5, mean=24.5%; 20e6 cells/ml: CS10, mean=16.8%, CS5, mean=37.7%). N=3.

FIGS. 6A-6C show data collected from the DeltaCel Process. FIG. 6A shows post-thaw cell viability. FIGS. 6B-6C show cytoxicity/potency of DeltaCel Process harvested at Day 8. N=2.

FIGS. 7A-7C show transduced human gamma delta T cells expressing chPD1 receptor and expanded in vitro. FIG. 7A shows fold-expansion of non-transduced (squares), or chPD1-expressing γδ T cells (triangles), was measured in vitro. FIGS. 7B-7C show human γδ T cells were transduced to express the chPD1 receptor. Purity of chPD1 γδ T cells (FIG. 7B) and cell surface expression of PD1 (FIG. 7C) was measured by flow cytometry. Cells were stained with anti-PD-1 antibodies or isotype control and were analyzed using flow cytometry. Non-transduced γδ T cells were used as a control. Data are representative of three separate donors.

FIG. 8 shows expression of PD-L1 on human cancer cell lines and healthy cells. Expression of PD-L1 was determined on human cancer cell lines and healthy cells using anti-PD-L1 or isotype control antibodies. Cells were analyzed using flow cytometry. SKOV-3 cells were incubated with TNFα or without TNFα for 48 hours before flow cytometry analysis was performed. Data are representative of one experiment.

FIGS. 9A-9H shows human gamma delta chPD1-expressing T cells lyse tumor cells. Non-transduced (circles) and chPD1 γδ T cells (squares) were used as effector cells with tumor targets at the indicated E:T ratios (1:1, 5:1, 25:1; FIGS. 9A-9D) and cell lysis was measured after 5 hours using an LDH assay or (4:1, 0.5:1, 0.25:1; FIGS. 9E-9H) and cell lysis was measured after 60 hours using flow cytometry. Specific lysis data is shown from HCC827 cells (FIGS. 9A and 9E), NCI-H226 cells (FIGS. 9B and 9F), and SKOV3 cells in the presence (FIGS. 9C and 9G) and absence (FIGS. 9D and 9H) of TNFα. ChPD1 T cells had significantly higher specific lysis of tumor cell lines at all E:T ratios compared to non-transduced T cells (*p<0.001). Data are presented as mean+SD and are representative of three separate donors.

FIG. 10 shows expression of PD-L1 on NCI-H226 tumor cells and primary cells from normal tissues. Anti-PD-L1 antibodies or isotype control antibodies show high expression of PD-L1 in the NCI-H226 cancer cell line. PD-L1 expression in normal healthy cells was variable with human bone marrow mononuclear cells, peripheral blood mononuclear cells, peripheral blood CD14+ monocytes, CD19+ B cells, hepatocytes, skeletal muscle cells, and normal astrocytes showing moderate cell surface expression of PD-L1 and intestinal epithelial cells and myofibroblasts showing low cell surface expression of PD-L1.

FIG. 11 shows human gamma delta chPD1-expressing T cells do not lyse normal human cells. Non-transduced and chPD1-GDT were used as effector cells with NCI-H226 tumor cells and healthy human cell targets at 4:1 ET ratio. Cell lysis was measured using the eFluor-based flow-cytometry assay 60 hours post incubation. Data are presented as mean±SD of triplicate values from three donors. *, p<0.05. There was no difference in the lysis of normal primary cells by chPD1 gdT cells, non-transduced gdT cells, or the spontaneous cell death rate (measured with target cells without effectors).

FIGS. 12A-12B show scheme of the bioinformatic analysis for the detection of cancer-associated isoforms. FIG. 12A: a cancer tissue distribution of the IsoMSLN transcripts per million reads (TPM) values is presented, showing the greatest upregulation in ovarian cancer (OC) followed by malignant pleural mesothelioma (MPM). FIG. 12B: CancerSplice multi sequence alignment tool (MSA) tool predicts the protein products of the detected transcripts variants and shows an alignment to highlight the amino acid differences that are targetable by antibodies or CAR molecules (bolded sequence=canonical form, underlined sequence=cancer-associated isoform, other sequences=protein products of minor transcripts with low TPM values).

FIGS. 13A-13C show detection of cell surface expression of MSLN isoform 1 and 2 on the tumor cell surface by anti-isoMSLN-specific antibodies. FIG. 13A: the binding curve of different MSLN antibody clones in MSLN isoform 1 or isoform 2 overexpressing 293T cells. MSLN isoform 1 or 2 were transiently expressed in Lenti-X 293T cells; 48 hrs after transfection the cells were harvested and stained for different anti-MSLN antibody or mIgG1 isotype control (with different antibody concentration). MSLN-expressing cells were gated based on EGFP expression. The antibody binding MFI is plot along with the antibody concentration. FIG. 13B: flow cytometry staining of NCI H226 and Hela tumor cell lines with different anti-MSLN antibody clones. FIG. 13C: binding curve of increasing concentration of anti-IsoMSLN antibody clones to NCI H226 cells. The positive percentage of MSLN is plotted versus the antibody concentration.

FIG. 14 shows detection of MSLN isoforms in mesothelioma and ovarian cancer TMA.

FIGS. 15A-15C shows anti-IsoMSLN CAR transduction in human γδ T cells. FIG. 15A shows a schematic overview of anti-IsoMSLN CAR design. The CAR binder is scFv sequence derived from anti-IsoMSLN antibody, with the human CD8 membrane signal peptide and human CD34 epitope peptide tag in the N-terminal and CD8a stalk region, CD8a transmembrane domain and the CD28 cytoplasmatic region, and the CD3z cytoplasmatic region in the C-terminal. The constructs were cloned into the pSFG retroviral vector backbone. FIG. 15B shows human 76 T purity and expansion. Purity of γδ T cells and cell surface expression of chimeric antigen receptor was measured by flow cytometry. Cells were stained with anti-CD34 antibodies or isotype control. Non-transduced γδ T cells were used as a control. FIG. 15C shows fold-expansion of IsoMSLN-γδ T cells measured during in vitro culture.

FIG. 16 shows target-specific killing of MSLN isoform 2-specific tumor cells by CAR-γδ T cells in vitro. Non-transduced (circles) and IsoMSLN-FA T cells (squares) were used as effector cells with tumor cell targets at the indicated E:T ratios and cell lysis was measured using flow cytometry. IsoMSLN-γδ T had significantly higher specific lysis of tumor cell lines at all E:T ratios compared to non-transduced T cells (* p<0.001). Data are presented as mean+SD and are representative of one experiment.

### DETAILED DESCRIPTION OF THE INVENTION

Provided herein are methods of making an enriched population of immune cells, such as gamma delta T-cells (gdT-cells). In aspects, the enriched population of immune cells can be modified, e.g., by mutations, insertions or deletions in one or more endogenous genes, by adding one or more exogenous genes. In aspects, the one or more exogenous genes express one or more of the binding molecules provided herein.

### I. Definitions

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The term “bisphosphonate” as used herein refers to a member of the class of pharmaceutical compounds that contain two phosphonate (PO(OH)₂) groups. The term bisphosphonate may be used interchangeably with diphosphonate. In embodiments, bisphosphonate may include, but not limited to zoledronate (zoledronic acid), clodronate, etidronate, alendronate, pamidronate, or neridronate.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. In embodiments, the nonspecific nucleic acid sequence does not encode a biological function. In embodiments, the nonspecific nucleic acid sequence is a scrambled nucleic acid sequence. A scrambled nucleic acid sequence as provided herein is a recombinant nucleic acid sequence that includes nucleotides randomly linked to each other in vitro. Scrambled nucleic acid sequences are commonly used in the art as control or reference sequences relative to the activity (biological function) of test nucleic acid sequences.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91% 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, 7-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-TUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., IsoMSLN) in other proteins with different numbering systems. For example, by performing a simple sequence alignment with a protein (e.g., IsoMSLN) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the residue to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence (e.g., a scrambled or non-specific nucleic acid sequence), to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3. In embodiments, the CDRs of the light chain are referred to as CDR1, CDR2, and CDR3 of VL and the CDRs of the heavy chain are referred to as CDR1, CDR2, and CDR3 of VH. See, for example the tables as provided herein.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. The term “light chain” is used according to its ordinary meaning in the biological arts, and refers to the polypeptide formed by a light chain variable domain (VL) and a light chain constant domain (CL). Likewise, the term “heavy chain” is used according to its ordinary meaning in the biological arts, and refers to the polypeptide formed by a heavy chain variable domain (VH) and one or more heavy chain constant domains (CH1, CH2, CH3).

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable domains of the heavy (VH) and light chain (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (e.g, variable region including domain VH and VL) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

As used herein, the term “binding molecule” is used in accordance with its plain and ordinary meaning and refers to a an agent, e.g., a polypeptide, antibody, antibody variant, antibody region or fragment thereof, capable of binding to another polypeptide or other molecule.

A “ligand” refers to an agent, e.g., a polypeptide or other molecule, capable of binding to a receptor or antibody, antibody variant, antibody region or fragment thereof.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “interleukin 7”, “IL7” or “IL-7” as used herein includes any of the recombinant or naturally-occurring forms of the interleukin 7 protein, or variants or homologs thereof that maintain IL-7 biological or structural function (e.g. within at least 50%, 80%, 90%, 95%, 96%, 97%, 98%, 99% or 100% biological or structural function compared to IL-7). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-7 protein. In embodiments, IL-7 is encoded by the gene IL7 (NCBI Gene ID: 3574). In embodiments, the IL-7 is substantially identical to the protein identified by the UniProt reference number Q5FBY3 or a variant or homolog having substantial identity thereto.

The term “interleukin 2”, “IL2” or “IL-2” as used herein includes any of the recombinant or naturally-occurring forms of the interleukin 2 protein, or variants or homologs thereof that maintain IL-2 biological or structural function (e.g. within at least 50%, 80%, 90%, 95%, 96%, 97%, 98%, 99% or 100% biological or structural function compared to IL-2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 protein. In embodiments, IL-2 is encoded by the gene IL2 (NCBI Gene ID: 3558). In embodiments, the IL-2 is substantially identical to the protein identified by the UniProt reference number P60568 or a variant or homolog having substantial identity thereto.

The term “isomesothelin” or “IsoMSLN” as used herein includes any of the recombinant or naturally-occurring forms of the mesothelin isoform-2 protein, or variants or homologs thereof that maintain IsoMSLN biological or structural function (e.g. within at least 50%, 80%, 90%, 95%, 96%, 97%, 98%, 99% or 100% biological or structural function compared to IsoMSLN). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IsoMSLN protein. In embodiments, the IsoMSLN is substantially identical to the protein identified by the UniProt reference number Q13421-3 or a variant or homolog having substantial identity thereto.

The term “PD1” or “PD-1” as used herein includes any of the recombinant or naturally-occurring forms of the programmed cell death protein 1, or variants or homologs thereof that maintain PD1 biological or structural function (e.g. within at least 50%, 80%, 90%, 95%, 96%, 97%, 98%, 99% or 100% biological or structural function compared to PD1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD1 protein. In embodiments, the PD1 is substantially identical to the protein identified by the UniProt reference number Q15116 or a variant or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids. A vector” may be any agent capable of delivering or maintaining nucleic acid in a host cell, and includes viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art. Transcription of the DNA encoding the polypeptides of the present invention by higher eukaryotes is increased by inserting an enhancer sequence into the vector. Enhancers are cis-acting elements of DNA, usually about from 10 to 300 by that act on a promoter to increase its transcription. Examples including the SV40 enhancer on the late side of the replication origin by 100 to 270, a cytomegalovirus early promoter enhancer, the polyoma enhancer on the late side of the replication origin, and adenovirus enhancers.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “cell surface molecule” as used herein, refers to a molecule wherein at least a portion of the molecule is expressed on the surface of a cell. In embodiments, the cell surface molecule spans the membrane of a cell including an extracellular portion and a transmembrane portion.

The term “peripheral blood mononuclear cell” or “PMBC” as used herein refers to a cell or cells that are typically found in the peripheral blood with a round nucleus. Any cell with a rounded nucleus normally found within the peripheral blood is considered a “peripheral blood mononuclear cell” or “PMBC”. In one embodiment, a PBMC is a “leukocyte”.

The term “leukocyte” or “white blood cell” as used herein refers to a cell or cells that are mononuclear and found in the peripheral blood. These cells are part of the immune system and fight against infection and disease. In embodiments, a leukocyte is a “lymphocyte”. In embodiments, a leukocyte is a “monocyte”.

The term “lymphocyte” as used herein refers to a cell or cells that are immune cells with a large nucleus found in lymphatic system of the body. In embodiments a lymphocyte is a “T-cell”, a “B cell” or a “Natural Killer (NK) cell”.

The term “T-cell” as used herein refers to a cell or cells that are part of the adaptive immune response and express a T-cell receptor (TCR) on their cell surface. In embodiments, a T-cell is an “alpha beta T-cell” or a “gamma delta T-cell”.

The term “alpha beta T-cell”, “abT-cell” or “αβ T-cell” as used herein refers to a cell or cells that express a TCR that includes an alpha TCR glycoprotein chain and a beta TCR glycoprotein chain. The term “gamma delta T-cell”, “gdT-cell” or “γδ T-cell” as used herein refers to a cell or cells that express a TCR that includes a gamma TCR glycoprotein chain and a delta TCR glycoprotein chain.

The term “monocyte” as used herein refers to a cell or cells that are amoeboid in appearance with nongranulated cytoplasms and part of the immune system.

The cells can be from any animal, including but not limited to any mammal, such as mouse, rat, canine, feline, bovine, equine, porcine, non-human and human primates. Mammalian cells particularly suitable for cultivation in the present media include peripheral blood mononuclear cells (PBMCs) of human origin, which may be primary cells derived from peripheral blood. In addition, transformed cells or established PMBC lines can also be used. The cells used herein can be normal, healthy cells. The cells can be from donor with a healthy immune system. In embodiments, the cells are not primary cells, such as cells from an established cell line, transformed cells, thawed cells from a previously frozen collection and the like.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution.

Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

As used herein, “chimeric antigen receptor” is used according to its conventional meaning in the art refers to a recombinant protein including an antibody region and a transmembrane region.

An “antibody region” as provided herein refers to a monovalent or multivalent protein moiety that forms part of an antibody. A person of ordinary skill in the art would therefor immediately recognize that the antibody region is a protein moiety capable of binding an antigen (epitope). Thus, the antibody region provided herein may include a domain of an antibody or fragment (e.g., Fab) thereof. In embodiments, the antibody region includes a variable light chain domain and a variable heavy chain domain. A “variable light chain domain” as provided herein refers to a polypeptide including a light chain variable (VL) region. In embodiments, the variable light chain domain is a light chain variable (VL) region. A “variable heavy chain domain” as provided herein refers to a polypeptide including a heavy chain variable (VH) region. In embodiments, the variable heavy chain domain is a heavy chain variable (VH) region.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein (e.g., the chimeric antigen receptor) including embodiments thereof are contemplated. Non-limiting examples of transmembrane domains include the transmembrane domains of CD28, CD8, CD4 or CD3-zeta.

In embodiments, the chimeric antigen receptor further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results expression by said T cell of proteins known in the art to characteristic of activated T cell (e.g., CTLA-4, PD-1, CD28, CD69). In embodiments, the intracellular T-cell signaling domain is a CD3 ζ intracellular T-cell signaling domain.

By “cell culture” or “culture” is meant the maintenance of the cells in an artificial, in vitro environment. The term “cell culture” also encompasses cultivating individual cells and tissues. The cells being cultured according to the present invention, whether primary or not, can be cultured and plated or suspended according to the disclosed conditions. The examples herein demonstrate at least one functional set of culture conditions that can be used in conjunction with the methods described herein. If not known, plating or suspension and culture conditions for a given animal cell type can be determined by one of ordinary skill in the art using only routine experimentation. Cells may or may not be plated onto the surface of culture vessels, and, if plated, attachment factors can be used to plate the cells onto the surface of culture vessels. If attachment factors are used, the culture vessels can be precoated with a natural, recombinant or synthetic attachment factor or factors or peptide fragments thereof, such as but not limited to collagen, fibronectin and natural or synthetic fragments thereof.

As used herein, the term “culturing T-cells” is used in accordance with its plain and ordinary meaning and refers to the process by which cells are grown under controlled conditions, generally outside their natural environment. After the cells of interest, herein T-cells, have been isolated from living tissue, they can subsequently be maintained under carefully controlled conditions. These conditions vary for each cell type, but generally consist of a suitable vessel with a substrate or medium that supplies the essential nutrients.

As used herein, the term “incubating” is used in accordance with its plain and ordinary meaning and refers to a process of contacting one or more components of a reaction with another component or components, under conditions and for sufficient time such that a desired reaction product is formed. The term “incubating” is used to describe a particular step in which a cell or group of cells is regulated. The incubating process may include regulating a particular temperature, reagent, or condition of the cell or group of cells.

As used herein, the term “media composition” is used in accordance with its plain and ordinary meaning and encompasses any gel or liquid created to support cellular growth in an artificial environment. A media composition plays an integral role in cell culture technology, supporting in vitro cellular research. It is the medium that supplies the nutrients necessary for cell cultures to survive and proliferate. The media composition also provides the correct osmolality and pH. There are a variety of different types of cell culture media that accommodate cells from mammals, plants, insects, bacteria, yeast, viruses, and more. The term media composition may be used interchangeably with cell culture medium, cell medium, or culture medium. In embodiments, media composition may include, but not limited to, any of the bisphosphonates, IL-7, and/or IL-2.

As used herein, the term “passaging cells” is used in accordance with its plain and ordinary meaning and refers to a process of removing cell culture medium and transfer of cells from a previous culture into fresh cell culture medium. This procedure allows for further propagation of the cell line. The term passaging cells may be used interchangeably with subculturing cells.

As used herein, the term “expanded culture” is used in accordance with its plain and ordinary meaning and refers to a population of cells that has been serially passaged from smaller cultures or volumes of cells into larger quantities of cells. In embodiments, the expanded culture is an expanded gd T-cell culture.

As used herein, the term “depleted culture” is used in accordance with its plain and ordinary meaning and refers to a cell culture in which a specified cell population is removed from the culture resulting in a population of cells that lacks the specified cell. In embodiments, the depleted culture is a monocyte-depleted culture. In embodiments, the depleted culture is an ab T-cell-depleted culture.

As used herein, the term “removing cells from a cell culture” is used in accordance with its plain and ordinary meaning and refers to a process in which a specified first subpopulation of cells is removed from a cell culture. There are a variety of different processes to remove a specified first subpopulation of cells from a cell culture which may include, but not limited to, addition of a pharmaceutical compound or tagged microbeads to the media composition. In embodiments, the pharmaceutical compound inhibits growth of the specified first subpopulation of cells. In embodiments, the pharmaceutical compound activates a second subpopulation of cells to target and kill the specified first subpopulation of cells. In embodiments, the pharmaceutical compound is a bisphosphonate. In further embodiments, the bisphosphonate is zoledronic acid. In embodiments, the tagged microbeads are magnetic and target the specified first population of cells. In embodiments, the magnetic microbeads bound to the specified first population of cells are removed from the cell culture using magnetism.

As used herein, the term “enriched culture” is used in accordance with its plain and ordinary meaning and refers to cell culture in which a population of cells has been cultured in conditions that inhibit or remove a non-preferred subpopulation of cells and results in the enrichment of a preferred subpopulation of cells. In embodiments, the enriched culture is a gd T-cell enriched culture.

As used herein, the term “suspending” is used in accordance with its plain and ordinary meaning and refers to the placement of cells in a liquid media for storage or growth. In embodiments, the cells are not adherent to a surface. In embodiments, the liquid media is cell culture media or cryopreservation media.

As used herein, “cancer antigen” refers to a molecule expressed on a cancer cell. In embodiments, the cancer antigen is expressed at a higher level relative to a standard control. IN embodiments, the cancer antigen is expressed on a healthy cell. A “standard control” can be the level of expression of the cancer antigen of a healthy cell. The standard control may be the expression level of the cancer antigen in a cell from a healthy subject (i.e. a subject that does not have cancer). The standard control may be the expression level of a non-cancerous cell derived from the same subject as the cancer antigen expressing cancer. In embodiments, the standard control is an expression level of a low cancer antigen or cancer antigen negative cancer cell. For example, the standard control can be the expression level of a biological sample comprising healthy cells (i.e. non-cancer cells). The standard control can be the expression level of cells from a subject that has already been treated for a cancer antigen expressing cancer. In instances, the control value can be obtained from the same subject (i.e. from a later-obtained sample, subsequent to treatment of the cancer antigen expressing cancer). The standard control can also represent an average expression level gathered from a population of similar subjects (i.e. healthy individuals with a similar medical background, same age, weight, etc.). In embodiments, the expression level of a cancer antigen is at least 2-fold higher than the expression level of a standard control. In embodiments, the expression level of a cancer antigen is at least 5, 10, 50, 100, 150, 200, 250, 300, 350, 400, 450, 500, or 1,000-fold higher than the expression level of a standard control. In embodiments, the expression level of a cancer antigen is 5, 10, 50, 100, 200, 300, 400, 500, 1,000, 10,000 or 100,000-fold higher than the expression level of a standard control.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. IsoMSLN protein). Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. IsoMSLN protein) relative to the activity or function of the protein in the absence of the inhibitor. In embodiments inhibition means negatively affecting (e.g. decreasing) the concentration or levels of IsoMSLN relative to the concentration or level of the protein in the absence of the inhibitor. In embodiments inhibition refers to reduction of a disease or symptoms of disease. In embodiments, inhibition refers to a reduction in the activity of IsoMSLN. Thus, inhibition includes, at least in part, partially or totally blocking stimulation, decreasing, preventing, or delaying activation, or inactivating, desensitizing, or down-regulating signal transduction or enzymatic activity or the amount of IsoMSLN. In embodiments, inhibition refers to a reduction of activity of v resulting from a direct interaction (e.g. an inhibitor binds to IsoMSLN). In embodiments, inhibition refers to a reduction of activity of IsoMSLN from an indirect interaction (e.g. an inhibitor binds to a protein that activates IsoMSLN, thereby preventing target protein activation).

Thus, the terms “inhibitor,” “repressor” or “antagonist” or “downregulator” interchangeably refer to a substance capable of detectably decreasing the expression or activity of a given gene or protein (e.g. IsoMSLN protein). The antagonist can decrease IsoMSLN expression or activity 10%, 20%, 30%, 40%, 50%, 60%, 70%, 80%, 90% or more in comparison to a control in the absence of the antagonist. In certain instances, IsoMSLN expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The cancer may refer to a solid tumor malignancy. Solid tumor malignancies include malignant tumors that may be devoid of fluids or cysts. For example, the solid tumor malignancy may include breast cancer, ovarian cancer, pancreatic cancer, cervical cancer, gastric cancer, renal cancer, head and neck cancer, bone cancer, skin cancer or prostate cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer,

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., breast cancer, lung cancer)) means that the disease (e.g. cancer) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

### II. Methods

Provided herein are, inter alia, methods for generating a T-cell culture enriched for gamma delta T-cells (gdT-cells). An example of a method of preparing a composition enriched in gdT cells, as provided herein, includes exposing a sample containing a mixed population of immune cells, such as white blood cells, to a bisphosphonate (e.g. zoledronic acid), IL-2 and IL-7, thereby obtaining an expanded population of gdT cells. In aspects, the expanded population can further be treated to deplete alpha beta T-cells in the population, thereby further enriching for the gdT-cells. The resulting gdT-cell composition can be used in immunotherapy or can be transduced to obtain a genetically modified gdT-cell as described elsewhere herein. Generally described methods are disclosed in International PCT application PCT/US2021/040365, filed Jul. 2, 2021, and in International PCT application PCT/US2021/059652, filed Dec. 2, 2021, and are incorporated by reference herein in their entirety.

In an aspect a method for generating a T-cell culture enriched for gamma delta T-cells (gd T-cells) is provided. The method includes: (a) contacting a population of immune cells with a first media composition thereby forming an initial cell culture, wherein the population of immune cells includes alpha beta T-cells (abT-cells), gdT-cells, and monocytes, wherein the ratio of abT-cells to gdT-cells in the population of immune cells is at least about 16:1, wherein the ratio of monocytes to gdT-cells is at least about 2:1, and wherein the first media composition comprises a bisphosphonate, interleukin-7 (IL-7) and interleukin-2 (IL-2); (b) incubating the initial cell culture for a first time period of about one to about three days, thereby forming a monocyte-depleted immune cell culture; (c) after the first time period, contacting the monocyte-depleted cell culture with interleukin-7 (IL-7) and interleukin-2 (IL-2), but not a bisphosphonate, thereby forming a monocyte-depleted cell culture in contact with a second media composition; (d) incubating said monocyte-depleted cell culture in contact with the second media composition for a second time period of about one to about three days; thereby forming an expanded gdT cell culture, wherein the ratio of abT-cells to gdT-cells in the expanded gdT cell culture is less than about 10:1; (e) removing abT-cells from the expanded gdT cell culture thereby forming an abT-cell-depleted culture; (f) replacing said second media composition with a third media composition comprising interleukin-7 (IL-7) and interleukin-2 (IL-2), wherein the third media composition does not comprise a bisphosphonate; and, (g) incubating the abT-cell-depleted culture for at least one day in contact with the third media composition, thereby forming a T-cell culture enriched for gdT-cells, wherein the ratio of abT-cells to gdT-cells in the T-cell culture enriched for gdT-cells is less than about 1:2.

The methods of manufacturing enriched gdT cell compositions, as provided herein, include conditions in which the cells are exposed to one or more cytokines whose activity is respectively mediated by all or a portion of IL-7 and IL-2 receptors and a bisphosphonate (e.g. zoledronic acid). Any source of immune cells can be used in the method provided herein. In certain aspects, the conditions include exposure to IL-7, IL-2 and a bisphosphonate (e.g. zoledronic acid). Without being bound by theory, it is believed that IL-7 and IL-2 whose activity is respectively mediated by all or a portion of IL-7 and IL-2 receptors can preserve the potential of the gdT cells by reducing exhaustion of the cells. Without being bound by theory, it is believed that a bisphosphonate (e.g. zoledronic acid) can cause monocytes to activate gdT-cells to expand in number. Without being bound by theory, it is believed that because the activated gdT-cells target and kill monocytes within three days, it is unnecessary to provide additional bisphosphonate to the expanded gdT-cell culture after the first time period. Furthermore, without being bound by theory, because bisphosphonate may be inhibitory to gdT-cell expansion, it is believed that by avoiding introducing bisphosphonate at later times a gdT-cell viability and expansion is improved.

In aspects, the methods provided herein do not include the use of feeder cells.

In embodiments, the ratio of abT-cells to gdT-cells in the population of immune cells is at least about 16:1 In embodiments, the ratio of abT-cells to gdT-cells in the population of immune cells is at least about 17:1 In embodiments, the ratio of abT-cells to gdT-cells in the population of immune cells is at least about 18:1 In embodiments, the ratio of abT-cells to gdT-cells in the population of immune cells is at least about 19:1 In embodiments, the ratio of abT-cells to gdT-cells in the population of immune cells is at least about 20:1 In embodiments, the ratio of abT-cells to gdT-cells in the population of immune cells is at least about 25:1 In embodiments, the ratio of abT-cells to gdT-cells in the population of immune cells is at least about 50:1 In embodiments, the ratio of abT-cells to gdT-cells in the population of immune cells is at least about 100:1 In embodiments, the ratio of abT-cells to gdT-cells in the population of immune cells is at least about 150:1

In embodiments, the ratio of abT-cells to gdT-cells in the population of immune cells is at least 16:1 In embodiments, the ratio of abT-cells to gdT-cells in the population of immune cells is at least 17:1 In embodiments, the ratio of abT-cells to gdT-cells in the population of immune cells is at least 18:1 In embodiments, the ratio of abT-cells to gdT-cells in the population of immune cells is at least 19:1 In embodiments, the ratio of abT-cells to gdT-cells in the population of immune cells is at least 20:1 In embodiments, the ratio of abT-cells to gdT-cells in the population of immune cells is at least 25:1 In embodiments, the ratio of abT-cells to gdT-cells in the population of immune cells is at least 50:1 In embodiments, the ratio of abT-cells to gdT-cells in the population of immune cells is at least 100:1 In embodiments, the ratio of abT-cells to gdT-cells in the population of immune cells is at least 150:1

In embodiments, the ratio of abT-cells to gdT-cells in the population of immune cells is 16:1 In embodiments, the ratio of abT-cells to gdT-cells in the population of immune cells is 17:1 In embodiments, the ratio of abT-cells to gdT-cells in the population of immune cells is 18:1 In embodiments, the ratio of abT-cells to gdT-cells in the population of immune cells is 19:1 In embodiments, the ratio of abT-cells to gdT-cells in the population of immune cells is at least about 20:1 In embodiments, the ratio of abT-cells to gdT-cells in the population of immune cells is 25:1 In embodiments, the ratio of abT-cells to gdT-cells in the population of immune cells is 50:1 In embodiments, the ratio of abT-cells to gdT-cells in the population of immune cells is 100:1 In embodiments, the ratio of abT-cells to gdT-cells in the population of immune cells is 150:1

In embodiments, the ratio of monocytes to gdT-cells is at least about 2:1. In embodiments, the ratio of monocytes to gdT-cells is at least about 3:1. In embodiments, the ratio of monocytes to gdT-cells is at least about 5:1. In embodiments, the ratio of monocytes to gdT-cells is at least about 5:1. In embodiments, the ratio of monocytes to gdT-cells is at least about 10:1. In embodiments, the ratio of monocytes to gdT-cells is at least about 20:1. In embodiments, the ratio of monocytes to gdT-cells is at least about 30:1. In embodiments, the ratio of monocytes to gdT-cells is at least about 40:1. In embodiments, the ratio of monocytes to gdT-cells is at least about 50:1.

In embodiments, the ratio of monocytes to gdT-cells is at least 2:1. In embodiments, the ratio of monocytes to gdT-cells is at least 3:1. In embodiments, the ratio of monocytes to gdT-cells is at least 5:1. In embodiments, the ratio of monocytes to gdT-cells is at least 5:1. In embodiments, the ratio of monocytes to gdT-cells is at least 10:1. In embodiments, the the ratio of monocytes to gdT-cells is at least 0:1. In embodiments, the ratio of monocytes to gdT-cells is at least 30:1. In embodiments, the ratio of monocytes to gdT-cells is at least 40:1. In embodiments, the ratio of monocytes to gdT-cells is at least 50:1.

In embodiments, the ratio of monocytes to gdT-cells is 2:1. In embodiments, the ratio of monocytes to gdT-cells is 3:1. In embodiments, the ratio of monocytes to gdT-cells is 5:1. In embodiments, the ratio of monocytes to gdT-cells is 5:1. In embodiments, the ratio of monocytes to gdT-cells is 10:1. In embodiments, the ratio of monocytes to gdT-cells is at least about 20:1. In embodiments, the ratio of monocytes to gdT-cells is 30:1. In embodiments, the ratio of monocytes to gdT-cells is 40:1. In embodiments, the ratio of monocytes to gdT-cells is 50:1.

In embodiments, the concentration of bisphosphonate is about 1 μM to about 5 μM. In embodiments, the concentration of bisphosphonate is about 2 μM to about 5 μM. In embodiments, the concentration of bisphosphonate is about 3 M to about 5 μM. In embodiments, the concentration of bisphosphonate is about 4 μM to about 5 μM.

In embodiments, the concentration of bisphosphonate is about 1 μM to about 4 μM. In embodiments, the concentration of bisphosphonate is about 1 μM to about 3 μM. In embodiments, the concentration of bisphosphonate is about 1 μM to about 2 μM.

In embodiments, the concentration of bisphosphonate is 1 μM to 5 μM. In embodiments, the concentration of bisphosphonate is 2 μM to 5 μM. In embodiments, the concentration of bisphosphonate is 3 μM to 5 μM. In embodiments, the concentration of bisphosphonate is 4 μM to 5 M.

In embodiments, the concentration of bisphosphonate is 1 M to 4 μM. In embodiments, the concentration of bisphosphonate is 1 μM to 3 μM. In embodiments, the concentration of bisphosphonate is 1 μM to 2 μM.

In embodiments, the concentration of IL-7 is about 1 U/mL to about 250 U/mL. In embodiments, the concentration of IL-7 is about 2 U/mL to about 250 U/mL. In embodiments, the concentration of IL-7 is about 5 U/mL to about 250 U/mL. In embodiments, the concentration of IL-7 is about 10 U/mL to about 250 U/mL. In embodiments, the concentration of IL-7 is about 20 U/mL to about 250 U/mL. In embodiments, the concentration of IL-7 is about 30 U/mL to about 250 U/mL. In embodiments, the concentration of IL-7 is about 40 U/mL to about 250 U/mL. In embodiments, the concentration of IL-7 is about 50 U/mL to about 250 U/mL. In embodiments, the concentration of IL-7 is about 100 U/mL to about 250 U/mL. In embodiments, the concentration of IL-7 is about 150 U/mL to about 250 U/mL. In embodiments, the concentration of IL-7 is about 200 U/mL to about 250 U/mL.

In embodiments, the concentration of IL-7 is about 1 U/mL to about 200 U/mL. In embodiments, the concentration of IL-7 is about 1 U/mL to about 150 U/mL. In embodiments, the concentration of IL-7 is about 1 U/mL to about 100 U/mL. In embodiments, the concentration of IL-7 is about 1 U/mL to about 50 U/mL. In embodiments, the concentration of IL-7 is about 1 U/mL to about 40 U/mL. In embodiments, the concentration of IL-7 is about 1 U/mL to about 30 U/mL. In embodiments, the concentration of IL-7 is about 1 U/mL to about 20 U/mL. In embodiments, the concentration of IL-7 is about 1 U/mL to about 10 U/mL. In embodiments, the concentration of IL-7 is about 1 U/mL to about 5 U/mL. In embodiments, the concentration of IL-7 is about 1 U/mL to about 2 U/mL.

In embodiments, the concentration of IL-7 is 1 U/mL to 250 U/mL. In embodiments, the concentration of IL-7 is 2 U/mL to 250 U/mL. In embodiments, the concentration of IL-7 is 5 U/mL to 250 U/mL. In embodiments, the concentration of IL-7 is 10 U/mL to 250 U/mL. In embodiments, the concentration of IL-7 is 20 U/mL to 250 U/mL. In embodiments, the concentration of IL-7 is 30 U/mL to 250 U/mL. In embodiments, the concentration of IL-7 is 40 U/mL to 250 U/mL. In embodiments, the concentration of IL-7 is 50 U/mL to 250 U/mL. In embodiments, the concentration of IL-7 is 100 U/mL to 250 U/mL. In embodiments, the concentration of IL-7 is 150 U/mL to 250 U/mL. In embodiments, the concentration of IL-7 is 200 U/mL to 250 U/mL.

In embodiments, the concentration of IL-7 is 1 U/mL to 200 U/mL. In embodiments, the concentration of IL-7 is 1 U/mL to 150 U/mL. In embodiments, the concentration of IL-7 is 1 U/mL to 100 U/mL. In embodiments, the concentration of IL-7 is 1 U/mL to 50 U/mL. In embodiments, the concentration of IL-7 is 1 U/mL to 40 U/mL. In embodiments, the concentration of IL-7 is 1 U/mL to 30 U/mL. In embodiments, the concentration of IL-7 is 1 U/mL to 20 U/mL. In embodiments, the concentration of IL-7 is 1 U/mL to 10 U/mL. In embodiments, the concentration of IL-7 is 1 U/mL to 5 U/mL. In embodiments, the concentration of IL-7 is 1 U/mL to 2 U/mL.

In embodiments, the concentration of IL-2 is about 1 U/mL to about 300 U/mL. In embodiments, the concentration of IL-2 is about 2 U/mL to about 300 U/mL. In embodiments, the concentration of IL-2 is about 5 U/mL to about 300 U/mL. In embodiments, the concentration of IL-2 is about 10 U/mL to about 300 U/mL. In embodiments, the concentration of IL-2 is about 20 U/mL to about 300 U/mL. In embodiments, the concentration of IL-2 is about 30 U/mL to about 300 U/mL. In embodiments, the concentration of IL-2 is about 40 U/mL to about 300 U/mL. In embodiments, the concentration of IL-2 is about 50 U/mL to about 300 U/mL. In embodiments, the concentration of IL-2 is about 100 U/mL to about 300 U/mL. In embodiments, the concentration of IL-2 is about 150 U/mL to about 300 U/mL. In embodiments, the concentration of IL-2 is about 200 U/mL to about 300 U/mL. In embodiments, the concentration of IL-2 is about 250 U/mL to about 300 U/mL.

In embodiments, the concentration of IL-2 is about 1 U/mL to about 250 U/mL. In embodiments, the concentration of IL-2 is about 1 U/mL to about 200 U/mL. In embodiments, the concentration of IL-2 is about 1 U/mL to about 150 U/mL. In embodiments, the concentration of IL-2 is about 1 U/mL to about 100 U/mL. In embodiments, the concentration of IL-2 is about 1 U/mL to about 50 U/mL. In embodiments, the concentration of IL-2 is about 1 U/mL to about 40 U/mL. In embodiments, the concentration of IL-2 is about 1 U/mL to about 30 U/mL. In embodiments, the concentration of IL-2 is about 1 U/mL to about 20 U/mL. In embodiments, the concentration of IL-2 is about 1 U/mL to about 10 U/mL. In embodiments, the concentration of IL-2 is about 1 U/mL to about 5 U/mL. In embodiments, the concentration of IL-2 is about 1 U/mL to about 2 U/mL.

In embodiments, the concentration of IL-2 is 1 U/mL to 300 U/mL. In embodiments, the concentration of IL-2 is 1 U/mL to 300 U/mL. In embodiments, the concentration of IL-2 is 2 U/mL to 250 U/mL. In embodiments, the concentration of IL-2 is 5 U/mL to 300 U/mL. In embodiments, the concentration of IL-2 is 10 U/mL to 300 U/mL. In embodiments, the concentration of IL-2 is 20 U/mL to 300 U/mL. In embodiments, the concentration of IL-2 is 30 U/mL to 300 U/mL. In embodiments, the concentration of IL-2 is 40 U/mL to 300 U/mL. In embodiments, the concentration of IL-2 is 50 U/mL to 300 U/mL. In embodiments, the concentration of IL-2 is 100 U/mL to 2300 50 U/mL. In embodiments, the concentration of IL-2 is 150 U/mL to 250 U/mL. In embodiments, the concentration of IL-2 is 200 U/mL to 300 U/mL. In embodiments, the concentration of IL-2 is 250 U/mL to 300 U/mL.

In embodiments, the concentration of IL-2 is 1 U/mL to 250 U/mL. In embodiments, the concentration of IL-2 is 1 U/mL to 200 U/mL. In embodiments, the concentration of IL-2 is 1 U/mL to 150 U/mL. In embodiments, the concentration of IL-2 is 1 U/mL to 100 U/mL. In embodiments, the concentration of IL-2 is 1 U/mL to 50 U/mL. In embodiments, the concentration of IL-2 is 1 U/mL to 40 U/mL. In embodiments, the concentration of IL-2 is 1 U/mL to 30 U/mL. In embodiments, the concentration of IL-2 is 1 U/mL to 20 U/mL. In embodiments, the concentration of IL-2 is 1 U/mL to 10 U/mL. In embodiments, the concentration of IL-2 is 1 U/mL to 5 U/mL. In embodiments, the concentration of IL-2 is 1 U/mL to 2 U/mL.

In embodiments, the initial cell culture is incubated for a first time period of about one to about three days. In embodiments, the initial cell culture is incubated for a first time period of one to three days. In embodiments, the initial cell culture is incubated for a first time period of about one day. In embodiments, the initial cell culture is incubated for a first time period of one day. In embodiments, the initial cell culture is incubated for a first time period of about two days. In embodiments, the initial cell culture is incubated for a first time period of two days. In embodiments, the initial cell culture is incubated for a first time period of about three days. In embodiments, the initial cell culture is incubated for a first time period of three days.

In embodiments, the monocyte-depleted culture is incubated in contact with the second media composition for a second time period of about one to about three days. In embodiments, the monocyte-depleted culture is incubated in contact with the second media composition for a second time period of one to three days. In embodiments, the monocyte-depleted culture is incubated in contact with the second media composition for a second time period of about one day. In embodiments, the monocyte-depleted culture is incubated in contact with the second media composition for a second time period of one day. In embodiments, the monocyte-depleted culture is incubated in contact with the second media composition for a second time period of about two days. In embodiments, the monocyte-depleted culture is incubated in contact with the second media composition for a second time period of two days. In embodiments, the monocyte-depleted culture is incubated in contact with the second media composition for a second time period of about three days. In embodiments, the monocyte-depleted culture is incubated in contact with the second media composition for a second time period of three days.

In embodiments, the ratio of abT-cells to gdT-cells in the expanded gdT cell culture is less than about 10:1. In embodiments, the ratio of abT-cells to gdT-cells in the expanded gdT cell culture is less than about 5:1. In embodiments, the ratio of abT-cells to gdT-cells in the expanded gdT cell culture is less than about 1:1. In embodiments, the ratio of abT-cells to gdT-cells in the expanded gdT cell culture is less than about 1:5. In embodiments, the ratio of abT-cells to gdT-cells in the expanded gdT cell culture is less than about 1:10. In embodiments, the ratio of abT-cells to gdT-cells in the expanded gdT cell culture is less than about 1:50. In embodiments, the ratio of abT-cells to gdT-cells in the expanded gdT cell culture is less than about 1:100. In embodiments, the ratio of abT-cells to gdT-cells in the expanded gdT cell culture is less than about 1:500. In embodiments, the ratio of abT-cells to gdT-cells in the expanded gdT cell culture is less than about 1:1000.

In embodiments, the ratio of abT-cells to gdT-cells in the expanded gdT cell culture is less than 10:1. In embodiments, the ratio of abT-cells to gdT-cells in the expanded gdT cell culture is less than 5:1. In embodiments, the ratio of abT-cells to gdT-cells in the expanded gdT cell culture is less than 1:1. In embodiments, the ratio of abT-cells to gdT-cells in the expanded gdT cell culture is less than 1:5. In embodiments, the ratio of abT-cells to gdT-cells in the expanded gdT cell culture is less than 1:10. In embodiments, the ratio of abT-cells to gdT-cells in the expanded gdT cell culture is less than 1:50. In embodiments, the ratio of abT-cells to gdT-cells in the expanded gdT cell culture is less than 1:100. In embodiments, the ratio of abT-cells to gdT-cells in the expanded gdT cell culture is less than 1:500. In embodiments, the ratio of abT-cells to gdT-cells in the expanded gdT cell culture is less than 1:1000.

In embodiments, the population of immune cells is obtained from peripheral blood. In embodiments, the population of immune cells is obtained by fresh leukapheresis.

In embodiments, the bisphosphonate is zoledronate (zoledronic acid), clodronate, etidronate, alendronate, pamidronate, or neridronate. In embodiments, the bisphosphonate is clodronate. In embodiments, the bisphosphonate is etidronate. In embodiments, the bisphosphonate is alendronate. In embodiments, the bisphosphonate is pamidronate. In embodiments, the bisphosphonate is neridronate.

In embodiments, the abT-cells are not removed prior to step (e).

In embodiments, the concentration of IL-7 is about 250 U/mL. In embodiments, the concentration of IL-7 is 250 U/mL.

In embodiments, the ratio of abT-cells to gdT-cells of the expanded gdT cell culture is less than about 5:1. In embodiments, the ratio of abT-cells to gdT-cells of the expanded gdT cell culture is less than 5:1.

In embodiments, the ratio of abT-cells to gdT-cells of the expanded gdT cell culture is less than about 1:1. In embodiments, the ratio of abT-cells to gdT-cells of the expanded gdT cell culture is less than 1:1.

In embodiments, the ratio of abT-cells to gdT-cells of the T-cell culture enriched for gdT-cells is less than about 1:5. In embodiments, the ratio of abT-cells to gdT-cells of the T-cell culture enriched for gdT-cells is less than 1:5.

In embodiments, the ratio of abT-cells to gdT-cells of the T-cell culture enriched for gdT-cells is less than about 1:10. In embodiments, the ratio of abT-cells to gdT-cells of the T-cell culture enriched for gdT-cells is less than 1:10.

In embodiments, the ratio of abT-cells to gdT-cells of the T-cell culture enriched for gdT-cells is less than about 1:50. In embodiments, the ratio of abT-cells to gdT-cells of the T-cell culture enriched for gdT-cells is less than 1:50.

In embodiments, the ratio of abT-cells to gdT-cells of the T-cell culture enriched for gdT-cells is less than about 1:100. In embodiments, the ratio of abT-cells to gdT-cells of the T-cell culture enriched for gdT-cells is less than 1:100.

In embodiments, the ratio of abT-cells to gdT-cells of the T-cell culture enriched for gdT-cells is less than about 1:500. In embodiments, the ratio of abT-cells to gdT-cells of the T-cell culture enriched for gdT-cells is less than 1:500.

In embodiments, the ratio of abT-cells to gdT-cells of the T-cell culture enriched for gdT-cells is less than about 1:1000. In embodiments, the ratio of abT-cells to gdT-cells of the T-cell culture enriched for gdT-cells is less than 1:1000.

In embodiments, the percentage of abT-cells of total cells in the T-cell culture enriched for gdT-cells is about 1% or less. In embodiments, the percentage of abT-cells of total cells in the T-cell culture enriched for gdT-cells is 1% or less.

In embodiments, the percentage of abT-cells of total cells in the T-cell culture enriched for gdT-cells is about 0% to about 1%. In embodiments, the percentage of abT-cells of total cells in the T-cell culture enriched for gdT-cells is about 0.1% to about 1%. In embodiments, the percentage of abT-cells of total cells in the T-cell culture enriched for gdT-cells is about 0.2% to about 1%. In embodiments, the percentage of abT-cells of total cells in the T-cell culture enriched for gdT-cells is about 0.3% to about 1%. In embodiments, the percentage of abT-cells of total cells in the T-cell culture enriched for gdT-cells is about 0.4% to about 1%. In embodiments, the percentage of abT-cells of total cells in the T-cell culture enriched for gdT-cells is about 0.5% to about 1%. In embodiments, the percentage of abT-cells of total cells in the T-cell culture enriched for gdT-cells is about 0.6% to about 1%. In embodiments, the percentage of abT-cells of total cells in the T-cell culture enriched for gdT-cells is about 0.7% to about 1%. In embodiments, the percentage of abT-cells of total cells in the T-cell culture enriched for gdT-cells is about 0.8% to about 1%. In embodiments, the percentage of abT-cells of total cells in the T-cell culture enriched for gdT-cells is about 0.9% to about 1%.

In embodiments, the percentage of abT-cells of total cells in the T-cell culture enriched for gdT-cells is about 0% to about 0.9%. In embodiments, the percentage of abT-cells of total cells in the T-cell culture enriched for gdT-cells is about 0% to about 0.8%. In embodiments, the percentage of abT-cells of total cells in the T-cell culture enriched for gdT-cells is about 0% to about 0.7%. In embodiments, the percentage of abT-cells of total cells in the T-cell culture enriched for gdT-cells is about 0% to about 0.6%. In embodiments, the percentage of abT-cells of total cells in the T-cell culture enriched for gdT-cells is about 0% to about 0.5%. In embodiments, the percentage of abT-cells of total cells in the T-cell culture enriched for gdT-cells is about 0% to about 0.4%. In embodiments, the percentage of abT-cells of total cells in the T-cell culture enriched for gdT-cells is about 0% to about 0.3%. In embodiments, the percentage of abT-cells of total cells in the T-cell culture enriched for gdT-cells is about 0% to about 0.2%. In embodiments, the percentage of abT-cells of total cells in the T-cell culture enriched for gdT-cells is about 0% to about 0.1%.

In embodiments, the percentage of abT-cells of total cells in the T-cell culture enriched for gdT-cells is 0% to 1%. In embodiments, the percentage of abT-cells of total cells in the T-cell culture enriched for gdT-cells is 0.1% to 1%. In embodiments, the percentage of abT-cells of total cells in the T-cell culture enriched for gdT-cells is 0.2% to 1%. In embodiments, the percentage of abT-cells of total cells in the T-cell culture enriched for gdT-cells is 0.3% to 1%. In embodiments, the percentage of abT-cells of total cells in the T-cell culture enriched for gdT-cells is 0.4% to 1%. In embodiments, the percentage of abT-cells of total cells in the T-cell culture enriched for gdT-cells is 0.5% to 1%. In embodiments, the percentage of abT-cells of total cells in the T-cell culture enriched for gdT-cells is 0.6% to 1%. In embodiments, the percentage of abT-cells of total cells in the T-cell culture enriched for gdT-cells is 0.7% to 1%. In embodiments, the percentage of abT-cells of total cells in the T-cell culture enriched for gdT-cells is 0.8% to 1%. In embodiments, the percentage of abT-cells of total cells in the T-cell culture enriched for gdT-cells is 0.9% to 1%.

In embodiments, the percentage of abT-cells of total cells in the T-cell culture enriched for gdT-cells is 0% to 0.9%. In embodiments, the percentage of abT-cells of total cells in the T-cell culture enriched for gdT-cells is 0% to 0.8%. In embodiments, the percentage of abT-cells of total cells in the T-cell culture enriched for gdT-cells is 0% to 0.7%. In embodiments, the percentage of abT-cells of total cells in the T-cell culture enriched for gdT-cells is 0% to 0.6%. In embodiments, the percentage of abT-cells of total cells in the T-cell culture enriched for gdT-cells is 0% to 0.5%. In embodiments, the percentage of abT-cells of total cells in the T-cell culture enriched for gdT-cells is 0% to 0.4%. In embodiments, the percentage of abT-cells of total cells in the T-cell culture enriched for gdT-cells is 0% to 0.3%. In embodiments, the percentage of abT-cells of total cells in the T-cell culture enriched for gdT-cells is 0% to 0.2%. In embodiments, the percentage of abT-cells of total cells in the T-cell culture enriched for gdT-cells is 0% to 0.1%.

In embodiments, the method further includes incubating the abT-cell-depleted culture 2, 3, 4, 5, or 6 days. In embodiments, the method further includes incubating the abT-cell-depleted culture 2 days. In embodiments, the method further includes incubating the abT-cell-depleted culture 3 days. In embodiments, the method further includes incubating the abT-cell-depleted culture 4 days. In embodiments, the method further includes incubating the abT-cell-depleted culture 5 days. In embodiments, the method further includes incubating the abT-cell-depleted culture 6 days.

In embodiments, the method further includes: (h) harvesting and cryopreserving said T-cell culture enriched for gdT-cells. In embodiments, the cryopreserving includes suspending the T-cell culture enriched for gdT-cells in a cryopreservation medium.

In embodiments, the cryopreservation medium includes between about 5% and about 10% dimethylsulfoxide (DMSO). In embodiments, the cryopreservation medium includes between about 6% and about 10% DMSO. In embodiments, the cryopreservation medium includes between about 7% and about 10% DMSO. In embodiments, the cryopreservation medium includes between about 8% and about 10% DMSO. In embodiments, the cryopreservation medium includes between about 9% and about 10% DMSO.

In embodiments, the cryopreservation medium includes between about 5% and about 9% DMSO. In embodiments, the cryopreservation medium includes between about 5% and about 8% DMSO. In embodiments, the cryopreservation medium includes between about 5% and about 7% DMSO. In embodiments, the cryopreservation medium includes between about 5% and about 6% DMSO.

In embodiments, the cryopreservation medium includes between 5% and 10% dimethylsulfoxide (DMSO). In embodiments, the cryopreservation medium includes between 6% and 10% DMSO. In embodiments, the cryopreservation medium includes between 7% and 10% DMSO. In embodiments, the cryopreservation medium includes between 8% and 10% DMSO. In embodiments, the cryopreservation medium includes between 9% and 10% DMSO.

In embodiments, the cryopreservation medium includes between 5% and 9% DMSO. In embodiments, the cryopreservation medium includes between 5% and 8% DMSO. In embodiments, the cryopreservation medium includes between 5% and 7% DMSO. In embodiments, the cryopreservation medium includes between 5% and 6% DMSO.

In another aspect a method for generating a gdT-cell expressing a Chimeric Antigen Receptor (CAR) is provided. The method includes introducing a nuclei acid encoding a CAR to a gdT-cell obtained as provided herein, including embodiments thereof.

In embodiments, the CAR includes an anti-IsoMSLN binding molecule. In embodiments, the CAR includes a chimeric PD1 binding molecule. Methods for generating a gdT-cell expressing a CARs including an anti-IsoMSLN binding molecule or generating a gdT-cell expressing including a chimeric PD1 binding molecule are disclosed in International PCT application PCT/US2021/040348, filed Jul. 2, 2021, in International PCT application PCT/US2021/040365, filed Jul. 2, 2021, and in International PCT application PCT/US2021/059652, filed Dec. 2, 2021, and are incorporated by reference herein in their entirety.

In certain implementations, a binding molecule can include a Heavy Chain Variable (VH) Domain and a Light Chain Variable (VL) Domain, or components thereof (e.g., complementarity-determining regions (CDRs) and/or framework (FR) regions) from the 1B6 antibody, depicted in the structural formula associated with SEQ ID NO:2 and SEQ ID NO:11 as recited for “Binding Molecule A” shown in the Sequence Table. Binding Molecule A can include the VH Domain of SEQ ID NO:2 and the VL Domain of SEQ ID NO:11, or CDRs thereof defined in the Sequence Table. A VH Domain of SEQ ID NO:2 when present in a Binding Molecule A polypeptide sometimes is encoded by a polynucleotide of SEQ ID NO:20 in a nucleic acid. A VL Domain of SEQ ID NO:11 when present in a Binding Molecule A polypeptide sometimes is encoded by a polynucleotide of SEQ ID NO:29 in a nucleic acid. Binding Molecule A also is referred to herein as a “1B6 antibody,” a “1B6 monoclonal antibody (mAb),” a “1B6 monoclonal antibody clone” and a “1B6 clone.”

In certain implementations, a binding molecule can include a Heavy Chain Variable (VH) Domain and a Light Chain Variable (VL) Domain, or components thereof (e.g., complementarity-determining regions (CDRs) and/or framework (FR) regions) from the 11C11 antibody, depicted in the structural formula associated with SEQ ID NO:38 and SEQ ID NO:47 as recited for “Binding Molecule B” shown in the Sequence Table. Binding Molecule B can include the VH Domain of SEQ ID NO:38 and the VL Domain of SEQ ID NO:47, or CDRs thereof defined in the Sequence Table. A VH Domain of SEQ ID NO:38 when present in a Binding Molecule B polypeptide sometimes is encoded by a polynucleotide of SEQ ID NO:56 in a nucleic acid. A VL Domain of SEQ ID NO:47 when present in a Binding Molecule B polypeptide sometimes is encoded by a polynucleotide of SEQ ID NO:65 in a nucleic acid. Binding Molecule B also is referred to herein as a “11C11 antibody,” a “11C11 monoclonal antibody (mAb),” a “11C11 monoclonal antibody clone” and a “11C11 clone.”

In certain implementations, a binding molecule having a structure of Formula F can include one or more polypeptide regions described for “Binding Molecule C” shown in the Sequence Table. The CAR structure set forth for Binding Molecule C includes the VH and VL domains of the 1B6 antibody (i.e., a VH domain of SEQ ID NO:2 (e.g., equivalent to SEQ ID NO:83) and a VL domain of SEQ ID NO:11 (equivalent to SEQ ID NO:87). A CAR molecule according to Binding Molecule C can include or is the polypeptide of SEQ ID NO:73 or SEQ ID NO:196. A polypeptide region described for Binding Molecule C and depicted in Formula F can be encoded by an associated polynucleotide shown for Binding Molecule C in the Sequence Table. A Binding Molecule C polypeptide (e.g., SEQ ID NO:73 or SEQ ID NO:196) sometimes is encoded by a polynucleotide of SEQ ID NO:74, and can be encoded by a plasmid described herein (e.g., pKB113 plasmid (also referred to as “pKB0113” and “a plasmid construct expressing the 1B6 scFv”)). A CAR molecule defined under Binding Molecule C also is referred to herein as a “1B6” CAR herein.

In certain implementations, a binding molecule having a structure of Formula F can include one or more polypeptide regions described for “Binding Molecule D” shown in the Sequence Table. The CAR structure set forth for Binding Molecule D includes the VH and VL domains of the 11C11 antibody (i.e., a VH domain of SEQ ID NO:38 (e.g., equivalent to SEQ ID NO:111) and a VL domain of SEQ ID NO:47 (equivalent to SEQ ID NO: 115). A CAR according to Binding Molecule D can include or is the polypeptide of SEQ ID NO:101 (e.g., where “X” is valine) or SEQ ID NO:197. A polypeptide region described for Binding Molecule D and depicted in Formula F can be encoded by an associated polynucleotide shown for Binding Molecule D in the Sequence Table. A Binding Molecule D polypeptide (e.g., SEQ ID NO:101 or SEQ ID NO:197) can be encoded by a polynucleotide of SEQ ID NO:102 (e.g., where “Z” is guanine and “Y” is thymine), and can be encoded by a plasmid described herein (e.g., pKB115 plasmid (also referred to as “pKB0115” and “a plasmid construct expressing the 11C11 scFv”)). A CAR molecule defined under Binding Molecule D also is referred to herein as a “11C11” CAR herein.

In certain implementations, a binding molecule having a structure of Formula F can include one or more polypeptide regions described for “Binding Molecule E” shown in the Sequence Table. The CAR structure set forth for Binding Molecule E includes the VH and VL domains of the 11C11 antibody (i.e., a VH domain of SEQ ID NO:38 (e.g., equivalent to SEQ ID NO:178) and a VL domain of SEQ ID NO:47 (equivalent to SEQ ID NO:182). A CAR according to Binding Molecule E can include or is the polypeptide of SEQ ID NO: 168 or SEQ ID NO: 198. A polypeptide region described for Binding Molecule E and depicted in Formula F can be encoded by an associated polynucleotide shown for Binding Molecule E in the Sequence Table. In instances where “X” is valine in SEQ ID NO:101 and SEQ ID NO:115, and where “Z” is guanine and “Y” is thymine in SEQ ID NO:102 and SEQ ID NO:116, the sequences of SEQ ID NOs:101-128 for “Binding Molecule D” effectively are the same as the sequences of SEQ ID NOs:168-195 for “Binding Molecule E,” respectively. A Binding Molecule E polypeptide (e.g., SEQ ID NO:168 or SEQ ID NO:198) can be encoded by a polynucleotide of SEQ ID NO:169, and can be encoded by a plasmid described herein (e.g., pKB115 plasmid (also referred to as “pKB0115” and “a plasmid construct expressing the 11C11 scFv”)). A CAR molecule defined under Binding Molecule E also is referred to herein as a “11C11” CAR herein.

In certain implementations, a binding molecule having a structure of Formula H can include one or more polypeptide regions described for “Chimeric PD1 Molecule A” shown in the Sequence Table. Chimeric PD1 Molecule A can include or is the polypeptide of SEQ ID NO:147 or SEQ ID NO:199. A polypeptide region described for Chimeric PD1 Molecule A and depicted in Formula H can be encoded by an associated polynucleotide shown for Chimeric PD1 Molecule A in the Sequence Table. A Chimeric PD1 Molecule A polypeptide (e.g., SEQ ID NO:147 or SEQ ID NO:199) sometimes is encoded by a polynucleotide of SEQ ID NO:146, and can be encoded by a plasmid described herein (e.g., HchPD1.pSFG plasmid described in FIG. 17). Chimeric PD1 Molecule A also is referred to herein as “chPD1,” “chPD1-DAP10 receptor” and “chPD1-DAP10 CAR.”

In certain implementations, a binding molecule having a structure of Formula J can include one or more polypeptide regions described for “Chimeric PD1 Molecule B” shown in the Sequence Table. Chimeric PD1 Molecule B can include or is the polypeptide of SEQ ID NO:167 or SEQ ID NO:200. A polypeptide region described for Chimeric PD1 Molecule B and depicted in Formula J can be encoded by an associated polynucleotide shown for Chimeric PD1 Molecule B in the Sequence Table. A Chimeric PD1 Molecule B polypeptide (e.g., SEQ ID NO: 167 or SEQ ID NO:200) sometimes is encoded by a polynucleotide of SEQ ID NO:166. In certain instances, a polynucleotide encoding Chimeric PD1 Molecule B (e.g., a polynucleotide of SEQ ID NO:166) can replace a polynucleotide encoding Chimeric PD1 Molecule A (e.g., a polynucleotide of SEQ ID NO:146). A Chimeric PD1 Molecule B polypeptide can be encoded by a plasmid described herein (e.g., pKB0174, a version of the HchPD1.pSFG plasmid described in FIG. 17 in which the polynucleotide encoding the Chimeric PD1 Molecule A polypeptide is replaced with a polynucleotide encoding the Chimeric PD1 Molecule B polypeptide).

### III. Cell Compositions

In another aspect is provided a T-cell culture enriched for gdT-cells as provided herein, including embodiments thereof.

In another aspect is provided a T-cell population expressing a Chimeric Antigen Receptor (CAR), including the T-cell culture enriched for gdT-cells as provided herein, including embodiments thereof. The gdT-cells include a nucleic acid encoding the CAR.

In embodiments, the CAR includes an anti-IsoMSLN binding molecule. In embodiments, the CAR includes a chimeric PD1 binding molecule. CARs including an anti-IsoMSLN binding molecule are disclosed in International PCT application PCT/US2021/040348, filed Jul. 2, 2021, in International PCT application PCT/US2021/040365, filed Jul. 2, 2021, and in International PCT application PCT/US2021/059652, filed Dec. 2, 2021, and are incorporated by reference herein in their entirety. CARs including a chimeric PD1 binding molecule are disclosed in International PCT application PCT/US2021/040365, filed Jul. 2, 2021, and in International PCT application PCT/US2021/059652, filed Dec. 2, 2021, and are incorporated by reference herein in their entirety.

In certain implementations, a binding molecule can include a Heavy Chain Variable (VH) Domain and a Light Chain Variable (VL) Domain, or components thereof (e.g., complementarity-determining regions (CDRs) and/or framework (FR) regions) from the 1B6 antibody, depicted in the structural formula associated with SEQ ID NO:2 and SEQ ID NO:11 as recited for “Binding Molecule A” shown in the Sequence Table. Binding Molecule A can include the VH Domain of SEQ ID NO:2 and the VL Domain of SEQ ID NO:11, or CDRs thereof defined in the Sequence Table. A VH Domain of SEQ ID NO:2 when present in a Binding Molecule A polypeptide sometimes is encoded by a polynucleotide of SEQ ID NO:20 in a nucleic acid. A VL Domain of SEQ ID NO: 1 when present in a Binding Molecule A polypeptide sometimes is encoded by a polynucleotide of SEQ ID NO:29 in a nucleic acid. Binding Molecule A also is referred to herein as a “1B6 antibody,” a “1B6 monoclonal antibody (mAb),” a “1B6 monoclonal antibody clone” and a “1B6 clone.”

In certain implementations, a binding molecule can include a Heavy Chain Variable (VH) Domain and a Light Chain Variable (VL) Domain, or components thereof (e.g., complementarity-determining regions (CDRs) and/or framework (FR) regions) from the 11C11 antibody, depicted in the structural formula associated with SEQ ID NO:38 and SEQ ID NO:47 as recited for “Binding Molecule B” shown in the Sequence Table. Binding Molecule B can include the VH Domain of SEQ ID NO:38 and the VL Domain of SEQ ID NO:47, or CDRs thereof defined in the Sequence Table. A VH Domain of SEQ ID NO:38 when present in a Binding Molecule B polypeptide sometimes is encoded by a polynucleotide of SEQ ID NO:56 in a nucleic acid. A VL Domain of SEQ ID NO:47 when present in a Binding Molecule B polypeptide sometimes is encoded by a polynucleotide of SEQ ID NO:65 in a nucleic acid. Binding Molecule B also is referred to herein as a “11C11 antibody,” a “11C11 monoclonal antibody (mAb),” a “11C11 monoclonal antibody clone” and a “11C11 clone.”

In certain implementations, a binding molecule having a structure of Formula F can include one or more polypeptide regions described for “Binding Molecule C” shown in the Sequence Table. The CAR structure set forth for Binding Molecule C includes the VH and VL domains of the 1B6 antibody (i.e., a VH domain of SEQ ID NO:2 (e.g., equivalent to SEQ ID NO:83) and a VL domain of SEQ ID NO:11 (equivalent to SEQ ID NO:87). A CAR molecule according to Binding Molecule C can include or is the polypeptide of SEQ ID NO:73 or SEQ ID NO:196. A polypeptide region described for Binding Molecule C and depicted in Formula F can be encoded by an associated polynucleotide shown for Binding Molecule C in the Sequence Table. A Binding Molecule C polypeptide (e.g., SEQ ID NO:73 or SEQ ID NO:196) sometimes is encoded by a polynucleotide of SEQ ID NO:74, and can be encoded by a plasmid described herein (e.g., pKB113 plasmid (also referred to as “pKB0113” and “a plasmid construct expressing the 1B6 scFv”)). A CAR molecule defined under Binding Molecule C also is referred to herein as a “1B6” CAR herein.

In certain implementations, a binding molecule having a structure of Formula F can include one or more polypeptide regions described for “Binding Molecule D” shown in the Sequence Table. The CAR structure set forth for Binding Molecule D includes the VH and VL domains of the 11C11 antibody (i.e., a VH domain of SEQ ID NO:38 (e.g., equivalent to SEQ ID NO:111) and a VL domain of SEQ ID NO:47 (equivalent to SEQ ID NO: 115). A CAR according to Binding Molecule D can include or is the polypeptide of SEQ ID NO:101 (e.g., where “X” is valine) or SEQ ID NO:197. A polypeptide region described for Binding Molecule D and depicted in Formula F can be encoded by an associated polynucleotide shown for Binding Molecule D in the Sequence Table. A Binding Molecule D polypeptide (e.g., SEQ ID NO:101 or SEQ ID NO:197) can be encoded by a polynucleotide of SEQ ID NO:102 (e.g., where “Z” is guanine and “Y” is thymine), and can be encoded by a plasmid described herein (e.g., pKB115 plasmid (also referred to as “pKB0115” and “a plasmid construct expressing the 11C11 scFv”)). A CAR molecule defined under Binding Molecule D also is referred to herein as a “11C11” CAR herein.

In certain implementations, a binding molecule having a structure of Formula F can include one or more polypeptide regions described for “Binding Molecule E” shown in the Sequence Table. The CAR structure set forth for Binding Molecule E includes the VH and VL domains of the 11C11 antibody (i.e., a VH domain of SEQ ID NO:38 (e.g., equivalent to SEQ ID NO:178) and a VL domain of SEQ ID NO:47 (equivalent to SEQ ID NO:182). A CAR according to Binding Molecule E can include or is the polypeptide of SEQ ID NO: 168 or SEQ ID NO:198. A polypeptide region described for Binding Molecule E and depicted in Formula F can be encoded by an associated polynucleotide shown for Binding Molecule E in the Sequence Table. In instances where “X” is valine in SEQ ID NO:101 and SEQ ID NO:115, and where “Z” is guanine and “Y” is thymine in SEQ ID NO: 102 and SEQ ID NO:116, the sequences of SEQ ID NOs:101-128 for “Binding Molecule D” effectively are the same as the sequences of SEQ ID NOs:168-195 for “Binding Molecule E,” respectively. A Binding Molecule E polypeptide (e.g., SEQ ID NO:168 or SEQ ID NO:198) can be encoded by a polynucleotide of SEQ ID NO:169, and can be encoded by a plasmid described herein (e.g., pKB115 plasmid (also referred to as “pKB0115” and “a plasmid construct expressing the 11C11 scFv”)). A CAR molecule defined under Binding Molecule E also is referred to herein as a “11C11” CAR herein.

In certain implementations, a binding molecule having a structure of Formula H can include one or more polypeptide regions described for “Chimeric PD1 Molecule A” shown in the Sequence Table. Chimeric PD1 Molecule A can include or is the polypeptide of SEQ ID NO:147 or SEQ ID NO:199. A polypeptide region described for Chimeric PD1 Molecule A and depicted in Formula H can be encoded by an associated polynucleotide shown for Chimeric PD1 Molecule A in the Sequence Table. A Chimeric PD1 Molecule A polypeptide (e.g., SEQ ID NO:147 or SEQ ID NO:199) sometimes is encoded by a polynucleotide of SEQ ID NO:146, and can be encoded by a plasmid described herein (e.g., HchPD1.pSFG plasmid described in FIG. 17). Chimeric PD1 Molecule A also is referred to herein as “chPD1,” “chPD1-DAP10 receptor” and “chPD1-DAP10 CAR.”

In certain implementations, a binding molecule having a structure of Formula J can include one or more polypeptide regions described for “Chimeric PD1 Molecule B” shown in the Sequence Table. Chimeric PD1 Molecule B can include or is the polypeptide of SEQ ID NO:167 or SEQ ID NO:200. A polypeptide region described for Chimeric PD1 Molecule B and depicted in Formula J can be encoded by an associated polynucleotide shown for Chimeric PD1 Molecule B in the Sequence Table. A Chimeric PD1 Molecule B polypeptide (e.g., SEQ ID NO:167 or SEQ ID NO:200) sometimes is encoded by a polynucleotide of SEQ ID NO:166. In certain instances, a polynucleotide encoding Chimeric PD1 Molecule B (e.g., a polynucleotide of SEQ ID NO:166) can replace a polynucleotide encoding Chimeric PD1 Molecule A (e.g., a polynucleotide of SEQ ID NO:146). A Chimeric PD1 Molecule B polypeptide can be encoded by a plasmid described herein (e.g., pKB0174, a version of the HchPD1.pSFG plasmid described in FIG. 17 in which the polynucleotide encoding the Chimeric PD1 Molecule A polypeptide is replaced with a polynucleotide encoding the Chimeric PD1 Molecule B polypeptide).

The Sequence Table is provided hereafter.

Sequence Table

| | | |
|---|---|---|
| | SEQ | |
| | ID | |
| Element | NO | Sequence and applicable structural formula |

Binding Molecule A

| | | |
|---|---|---|
| Heavy Chain Variable | 1 | MNFGFSLIFLVLVLKGVQCEVKLVESGGGLVKPGGSLKLSCEASGFTFSRYAM |
| (VH) Domain with optional | | SWVRHTPEKRLAWVASITFGGDPYYPDGLKGRFTISRDNTRNILYLQMNSLRS |
| leader sequence | | EDTAMYYCVRHESWFAYWGQGTLVTVSA |
| polypeptide | | (leader sequence in italic text; CDRs in bold text) |
| | | Structural formula: Nterm-(leader sequence)-FR1-CDR1- |
| | | FR2-CDR2-FR3-CDR3-FR4-Cterm |
| Heavy Chain Variable | 2 | EVKLVESGGGLVKPGGSLKLSCEASGFTFSRYAMSWVRHTPEKRLAWVASITF |
| (VH) Domain | | GGDPYYPDGLKGRFTISRDNTRNILYLQMNSLRSEDTAMYYCVRHESWFAYWG |
| polypeptide | | QGTLVTVSA |
| | | (CDRs in bold text) |
| | | Structural formula: Nterm-FR1-CDR1-FR2-CDR2-FR3-CDR3- |
| | | FR4-Cterm |
| VH Domain CDR1 | 3 | RYAMS |
| polypeptide | | |
| VH Domain CDR2 | 4 | SITFGGDPYYPDGLKG |
| polypeptide | | |
| VH Domain CDR3 | 5 | HESWFAY |
| polypeptide | | |
| VH Domain FR1 | 6 | EVKLVESGGGLVKPGGSLKLSCEASGFTFS |
| polypeptide | | |
| VH Domain FR2 | 7 | WVRHTPEKRLAWVA |
| polypeptide | | |
| VH Domain FR3 | 8 | RFTISRDNTRNILYLQMNSLRSEDTAMYYCVR |
| polypeptide | | |
| VH Domain FR4 | 9 | WGQGTLVTVSA |
| polypeptide | | |
| Light Chain Variable (VL) | 10 | MKLPVRLLVLMFWIPASSSDVLMTQTPLSLPVSLGEQVSISCRSSQTIVHTDG |
| Domain with optional | | NIYLEWYLQNPGQSPRLLIYKISNRFSGVPDRFSGSGSGTDFTLKISRVEAED |
| leader sequence | | LGIYYCFQASHVPYTFGGGTKLEIK |
| polypeptide | | (leader sequence in italic text; CDRs in bold text) |
| | | Structural formula: Nterm-(leader sequence)-FR1-CDR1- |
| | | FR2-CDR2-FR3-CDR3-FR4-Cterm |
| Light Chain Variable (VL) | 11 | DVLMTQTPLSLPVSLGEQVSISCRSSQTIVHTDGNIYLEWYLQNPGQSPRLLI |
| Domain | | YKISNRFSGVPDRFSGSGSGTDFTLKISRVEAEDLGIYYCFQASHVPYTFGGG |
| polypeptide | | TKLEIK |
| | | (CDRs in bold text) |
| | | Structural formula: Nterm-FR1-CDR1-FR2-CDR2-FR3-CDR3- |
| | | FR4-Cterm |
| VL Domain CDR1 | 12 | RSSQTIVHTDGNIYLE |
| polypeptide | | |
| VL Domain CDR2 | 13 | KISNRFS |
| polypeptide | | |
| VL Domain CDR3 | 14 | FQASHVPYT |
| polypeptide | | |
| VL Domain FR1 | 15 | DVLMTQTPLSLPVSLGEQVSISC |
| polypeptide | | |
| VL Domain FR2 | 16 | WYLQNPGQSPRLLIY |
| polypeptide | | |
| VL Domain FR3 | 17 | GVPDRFSGSGSGTDFTLKISRVEAEDLGIYYC |
| polypeptide | | |
| VL Domain FR4 | 18 | FGGGTKLEIK |
| polypeptide | | |
| Heavy Chain Variable | 19 | ATGAACTTCGGGTTCAGCTTGATTTTCCTTGTCCTTGTTTTAAAAGGTGTCCA |
| (VH) Domain with optional | | GTGTGAAGTGAAGCTGGTGGAGTCTGGGGGAGGCTTAGTGAAGCCTGGGGGGT |
| leader sequence in italic | | CCCTGAAACTCTCCTGTGAGGCCTCTGGGTTCACTTTCAGTCGCTATGCCATG |
| text; CDRs in bold text | | TCTTGGGTTCGCCATACTCCAGAGAAGAGACTGGCCTGGGTCGCATCCATAAC |
| polynucleotide | | TTTTGGTGGTGACCCGTATTATCCCGACGGTTTGAAGGGCCGATTCACCATCT |
| | | CCAGAGATAATACCAGGAACATCCTGTACCTGCAAATGAACAGTCTGAGGTCT |
| | | GAAGACACGGCCATGTATTATTGTGTACGACACGAGTCATGGTTTGCTTACTG |
| | | GGGCCAAGGGACTCTGGTCACTGTCTCTGCA |
| | | Structural formula: 5′-(leader sequence)-FR1-CDR1- |
| | | FR2-CDR2-FR3-CDR3-FR4-3′ |
| Heavy Chain Variable | 20 | GAAGTGAAGCTGGTGGAGTCTGGGGGAGGCTTAGTGAAGCCTGGGGGGTCCCT |
| (VH) Domain (CDRs in | | GAAACTCTCCTGTGAGGCCTCTGGGTTCACTTTCAGTCGCTATGCCATGTCTT |
| bold text) | | GGGTTCGCCATACTCCAGAGAAGAGACTGGCCTGGGTCGCATCCATAACTTTT |
| polynucleotide | | GGTGGTGACCCGTATTATCCCGACGGTTTGAAGGGCCGATTCACCATCTCCAG |
| | | AGATAATACCAGGAACATCCTGTACCTGCAAATGAACAGTCTGAGGTCTGAAG |
| | | ACACGGCCATGTATTATTGTGTACGACACGAGTCATGGTTTGCTTACTGGGGC |
| | | CAAGGGACTCTGGTCACTGTCTCTGCA |
| | | Structural formula: 5′-FR1-CDR1-FR2-CDR2-FR3-CDR3- |
| | | FR4-3′ |
| VH Domain CDR1 | 21 | CGCTATGCCATGTCT |
| polynucleotide | | |
| VH Domain CDR2 | 22 | TCCATAACTTTTGGTGGTGACCCGTATTATCCCGACGGTTTGAAGGGC |
| polynucleotide | | |
| VH Domain CDR3 | 23 | CACGAGTCATGGTTTGCTTAC |
| polynucleotide | | |
| VH Domain FR1 | 24 | GAAGTGAAGCTGGTGGAGTCTGGGGGAGGCTTAGTGAAGCCTGGGGGGTCCCT |
| polynucleotide | | GAAACTCTCCTGTGAGGCCTCTGGGTTCACTTTCAGT |
| VH Domain FR2 | 25 | TGGGTTCGCCATACTCCAGAGAAGAGACTGGCCTGGGTCGCA |
| polynucleotide | | |
| VH Domain FR3 | 26 | CGATTCACCATCTCCAGAGATAATACCAGGAACATCCTGTACCTGCAAATGAA |
| polynucleotide | | CAGTCTGAGGTCTGAAGACACGGCCATGTATTATTGTGTACGA |
| VH Domain FR4 | 27 | TGGGGCCAAGGGACTCTGGTCACTGTCTCTGCA |
| polynucleotide | | |
| Light Chain Variable (VL) | 28 | ATGAAGTTGCCTGTTAGGCTGTTGGTGCTGATGTTCTGGATTCCTGCTTCCAG |
| Domain with optional | | CAGTGATGTTTTGATGACCCAAACTCCACTCTCCCTGCCTGTCAGTCTTGGAG |
| leader sequence in italic | | AACAAGTCTCCATCTCTTGCAGATCTAGTCAGACCATTGTACATACTGATGGC |
| text; CDRs in bold text | | AACATCTATTTAGAATGGTACCTCCAGAACCCAGGCCAGTCTCCTAGGCTCCT |
| polynucleotide | | GATCTACAAAATTTCCAACCGATTTTCTGGGGTCCCTGACAGGTTCAGTGGCA |
| | | GTGGATCAGGGACAGATTTCACACTCAAGATCAGCAGAGTGGAGGCTGAGGAT |
| | | CTGGGAATTTATTACTGCTTTCAAGCTTCACATGTTCCGTACACGTTCGGAGG |
| | | GGGGACCAAGCTGGAAATAAAA |
| | | Structural formula: 5′-(leader sequence)-FR1-CDR1- |
| | | FR2-CDR2-FR3-CDR3-FR4-3′ |
| Light Chain Variable (VL) | 29 | GATGTTTTGATGACCCAAACTCCACTCTCCCTGCCTGTCAGTCTTGGAGAACA |
| Domain (CDRs in bold | | AGTCTCCATCTCTTGCAGATCTAGTCAGACCATTGTACATACTGATGGCAACA |
| text) | | TCTATTTAGAATGGTACCTCCAGAACCCAGGCCAGTCTCCTAGGCTCCTGATC |
| polynucleotide | | TACAAAATTTCCAACCGATTTTCTGGGGTCCCTGACAGGTTCAGTGGCAGTGG |
| | | ATCAGGGACAGATTTCACACTCAAGATCAGCAGAGTGGAGGCTGAGGATCTGG |
| | | GAATTTATTACTGCTTTCAAGCTTCACATGTTCCGTACACGTTCGGAGGGGGG |
| | | ACCAAGCTGGAAATAAAA |
| | | Structural formula: 5′-FR1-CDR1-FR2-CDR2-FR3-CDR3- |
| | | FR4-3′ |
| VL Domain CDR1 | 30 | AGATCTAGTCAGACCATTGTACATACTGATGGCAACATCTATTTAGAA |
| polynucleotide | | |
| VL Domain CDR2 | 31 | AAAATTTCCAACCGATTTTCT |
| polynucleotide | | |
| VL Domain CDR3 | 32 | TTTCAAGCTTCACATGTTCCGTACACG |
| polynucleotide | | |
| VL Domain FR1 | 33 | GATGTTTTGATGACCCAAACTCCACTCTCCCTGCCTGTCAGTCTTGGAGAACA |
| polynucleotide | | AGTCTCCATCTCTTGC |
| VL Domain FR2 | 34 | TGGTACCTCCAGAACCCAGGCCAGTCTCCTAGGCTCCTGATCTAC |
| polynucleotide | | |
| VL Domain FR3 | 35 | GGGGTCCCTGACAGGTTCAGTGGCAGTGGATCAGGGACAGATTTCACACTCAA |
| polynucleotide | | GATCAGCAGAGTGGAGGCTGAGGATCTGGGAATTTATTACTGC |
| VL Domain FR4 | 36 | TTCGGAGGGGGGACCAAGCTGGAAATAAAA |
| polynucleotide | | |

Binding Molecule B

| | | |
|---|---|---|
| Heavy Chain Variable | 37 | MNFGFSLIFLVLVLKGVQCEVKLVESGGGLVKPGGSLKVSCTASGFTFSRYAM |
| (VH) Domain with optional | | SWVRQTPERRLEWVASITFGGSAYYLDSVKGRFTISRDNAQNILYLQMNSLVS |
| leader sequence | | EDTAIYNCARHQPWFDYWGQGTLVTVSA |
| polypeptide | | (leader sequence in italic text; CDRs in bold text) |
| | | Structural formula: Nterm-(leader sequence)-FR1-CDR1- |
| | | FR2-CDR2-FR3-CDR3-FR4-Cterm |
| Heavy Chain Variable | 38 | EVKLVESGGGLVKPGGSLKVSCTASGFTFSRYAMSWVRQTPERRLEWVASITF |
| (VH) Domain | | GGSAYYLDSVKGRFTISRDNAQNILYLQMNSLVSEDTAIYNCARHQPWFDYWG |
| polypeptide | | QGTLVTVSA |
| | | (CDRs in bold text) |
| | | Structural formula: Nterm-FR1-CDR1-FR2-CDR2-FR3-CDR3- |
| | | FR4-Cterm |
| VH Domain CDR1 | 39 | RYAMS |
| polypeptide | | |
| VH Domain CDR2 | 40 | SITFGGSAYYLDSVKG |
| polypeptide | | |
| VH Domain CDR3 | 41 | HQPWFDY |
| polypeptide | | |
| VH Domain FR1 | 42 | EVKLVESGGGLVKPGGSLKVSCTASGFTFS |
| polypeptide | | |
| VH Domain FR2 | 43 | WVRQTPERRLEWVA |
| polypeptide | | |
| VH Domain FR3 | 44 | RFTISRDNAQNILYLQMNSLVSEDTAIYNCAR |
| polypeptide | | |
| VH Domain FR4 | 45 | WGQGTLVTVSA |
| polypeptide | | |
| Light Chain Variable (VL) | 46 | MNLPIRLLVLMFWIPASSSDVLLTQTPLSLPVSLGDQASISCRSSQNIXHSDG |
| Domain with optional | | DTYLDWFLQKPGQSPNLLIYKVSNRFSGVPDRFSGSGSGTDFTLKISRVEADD |
| leader sequence | | LGVYYCFQASHVPYTFGGGTKLEVK |
| polypeptide | | (leader sequence in italic text; CDRs in bold text) |
| | | Structural formula: Nterm-(leader sequence)-FR1-CDR1- |
| | | FR2-CDR2-FR3-CDR3-FR4-Cterm leader sequence-FR1-CDR1- |
| | | FR2-CDR2-FR3-CDR3-FR4) |
| | | X is I or V |
| Light Chain Variable (VL) | 47 | DVLLTQTPLSLPVSLGDQASISCRSSQNIXHSDGDTYLDWFLQKPGQSPNLLI |
| Domain | | YKVSNRFSGVPDRFSGSGSGTDFTLKISRVEADDLGVYYCFQASHVPYTFGGG |
| polypeptide | | TKLEVK |
| | | (CDRs in bold text) |
| | | Structural formula: Nterm-FR1-CDR1-FR2-CDR2-FR3-CDR3- |
| | | FR4-Cterm |
| | | X is I or V |
| VL Domain CDR1 | 48 | RSSQNIXHSDGDTYLD |
| polypeptide | | X is I or V |
| VL Domain CDR2 | 49 | KVSNRFS |
| polypeptide | | |
| VL Domain CDR3 | 50 | FQASHVPYT |
| polypeptide | | |
| VL Domain FR1 | 51 | DVLLTQTPLSLPVSLGDQASISC |
| polypeptide | | |
| VL Domain FR2 | 52 | WFLQKPGQSPNLLIY |
| polypeptide | | |
| VL Domain FR3 | 53 | GVPDRFSGSGSGTDFTLKISRVEADDIGVYYC |
| polypeptide | | |
| VL Domain FR4 | 54 | FGGGTKLEVK |
| polypeptide | | |
| Heavy Chain Variable | 55 | ATGAACTTCGGGTTCAGCTTGATTTTCCTTGTCCTTGTTTTAAAAGGTGTCCA |
| (VH) Domain with optional | | GTGCGAAGTGAAGCTGGTGGAGTCTGGGGGAGGCTTAGTGAAGCCTGGAGGGT |
| leader sequence in italic | | CCCTGAAAGTCTCCTGTACAGCCTCTGGATTCACTTTCAGTCGTTATGCCATG |
| text; CDRs in bold text | | TCTTGGGTTCGCCAGACTCCAGAGAGGAGGCTGGAGTGGGTCGCATCCATTAC |
| polynucleotide | | TTTTGGTGGTAGCGCCTACTATTTAGACAGTGTGAAGGGCCGATTCACCATCT |
| | | CCAGAGATAATGCCCAGAACATCCTGTACCTGCAAATGAACAGTCTGGTGTCT |
| | | GAGGACACGGCCATTTATAACTGTGCAAGACACCAGCCCTGGTTTGATTACTG |
| | | GGGCCAAGGGACTCTGGTCACTGTCTCTGCA |
| | | Structural formula: 5′-(leader sequence)-FR1-CDR1- |
| | | FR2-CDR2-FR3-CDR3-FR4-3′ |
| Heavy Chain Variable | 56 | GAAGTGAAGCTGGTGGAGTCTGGGGGAGGCTTAGTGAAGCCTGGAGGGTCCCT |
| (VH) Domain (CDRs in | | GAAAGTCTCCTGTACAGCCTCTGGATTCACTTTCAGTCGTTATGCCATGTCTT |
| bold text) | | GGGTTCGCCAGACTCCAGAGAGGAGGCTGGAGTGGGTCGCATCCATTACTTTT |
| polynucleotide | | GGTGGTAGCGCCTACTATTTAGACAGTGTGAAGGGCCGATTCACCATCTCCAG |
| | | AGATAATGCCCAGAACATCCTGTACCTGCAAATGAACAGTCTGGTGTCTGAGG |
| | | ACACGGCCATTTATAACTGTGCAAGACACCAGCCCTGGTTTGATTACTGGGGC |
| | | CAAGGGACTCTGGTCACTGTCTCTGCA |
| | | Structural formula: 5′-FR1-CDR1-FR2-CDR2-FR3-CDR3- |
| | | FR4-3′ |
| VH Domain CDR1 | 57 | CGTTATGCCATGTCT |
| polynucleotide | | |
| VH Domain CDR2 | 58 | TCCATTACTTTTGGTGGTAGCGCCTACTATTTAGACAGTGTGAAGGGC |
| polynucleotide | | |
| VH Domain CDR3 | 59 | CACCAGCCCTGGTTTGATTAC |
| polynucleotide | | |
| VH Domain FR1 | 60 | GAAGTGAAGCTGGTGGAGTCTGGGGGAGGCTTAGTGAAGCCTGGAGGGTCCCT |
| polynucleotide | | GAAAGTCTCCTGTACAGCCTCTGGATTCACTTTCAGT |
| VH Domain FR2 | 61 | TGGGTTCGCCAGACTCCAGAGAGGAGGCTGGAGTGGGTCGCA |
| polynucleotide | | |
| VH Domain FR3 | 62 | CGATTCACCATCTCCAGAGATAATGCCCAGAACATCCTGTACCTGCAAATGAA |
| polynucleotide | | CAGTCTGGTGTCTGAGGACACGGCCATTTATAACTGTGCAAGA |
| VH Domain FR4 | 63 | TGGGGCCAAGGGACTCTGGTCACTGTCTCTGCA |
| polynucleotide | | |
| Light Chain Variable (VL) | 64 | ATGAACTTGCCTATTAGGCTGTTGGTGCTGATGTTCTGGATTCCTGCTTCCAG |
| Domain with optional | | CAGTGATGTCTTGCTGACCCAGACTCCACTCTCCCTGCCTGTCAGTCTTGGAG |
| leader sequence in italic | | ATCAGGCCTCCATCTCTTGCAGATCTAGTCAGAACATTZTYCATAGTGATGGA |
| text; CDRs in bold text | | GACACCTATTTAGACTGGTTCCTGCAGAAACCAGGCCAGTCTCCAAACCTCCT |
| polynucleotide | | AATCTACAAAGTTTCCAACCGATTTTCTGGGGTCCCAGACAGGTTCAGTGGCA |
| | | GTGGATCAGGGACAGATTTCACCCTCAAGATCAGCAGAGTGGAGGCTGACGAT |
| | | CTGGGAGTTTATTACTGCTTTCAAGCTTCACATGTTCCGTACACGTTCGGAGG |
| | | GGGGACCAAGCTGGAAGTAAAA |
| | | Structural formula: Nterm- (leader sequence) -FR1-CDR1- |
| | | FR2-CDR2-FR3-CDR3-FR4-Cterm |
| | | Z is A or G; Y is A or T |
| Light Chain Variable (VL) | 65 | GATGTCTTGCTGACCCAGACTCCACTCTCCCTGCCTGTCAGTCTTGGAGATCA |
| Domain (CDRs in bold | | GGCCTCCATCTCTTGCAGATCTAGTCAGAACATTZTYCATAGTGATGGAGACA |
| text) | | CCTATTTAGACTGGTTCCTGCAGAAACCAGGCCAGTCTCCAAACCTCCTAATC |
| polynucleotide | | TACAAAGTTTCCAACCGATTTTCTGGGGTCCCAGACAGGTTCAGTGGCAGTGG |
| | | ATCAGGGACAGATTTCACCCTCAAGATCAGCAGAGTGGAGGCTGACGATCTGG |
| | | GAGTTTATTACTGCTTTCAAGCTTCACATGTTCCGTACACGTTCGGAGGGGGG |
| | | ACCAAGCTGGAAGTAAAA |
| | | Structural formula: 5′-FR1-CDR1-FR2-CDR2-FR3-CDR3- |
| | | FR4-3′ |
| | | Z is A or G; Y is A or T |
| VL Domain CDR1 | 66 | AGATCTAGTCAGAACATTZTYCATAGTGATGGAGACACCTATTTAGAC |
| polynucleotide | | Z is A or G; Y is A or T |
| VL Domain CDR2 | 67 | AAAGTTTCCAACCGATTTTCT |
| polynucleotide | | |
| VL Domain CDR3 | 68 | TTTCAAGCTTCACATGTTCCGTACACG |
| polynucleotide | | |
| VL Domain FR1 | 69 | GATGTCTTGCTGACCCAGACTCCACTCTCCCTGCCTGTCAGTCTTGGAGATCA |
| polynucleotide | | GGCCTCCATCTCTTGC |
| VL Domain FR2 | 70 | TGGTTCCTGCAGAAACCAGGCCAGTCTCCAAACCTCCTAATCTAC |
| polynucleotide | | |
| VL Domain FR3 | 71 | GGGGTCCCAGACAGGTTCAGTGGCAGTGGATCAGGGACAGATTTCACCCTCAA |
| polynucleotide | | GATCAGCAGAGTGGAGGCTGACGATCTGGGAGTTTATTACTGC |
| VL Domain FR4 | 72 | TTCGGAGGGGGGACCAAGCTGGAAGTAAAA |
| polynucleotide | | |

Binding Molecule C

| | | |
|---|---|---|
| CAR polypeptide | 73 | MALPVTALLLPLALLLHAARPGSELPTQGTFSNVSTNVSSPEVKLVESGGGLV |
| | | KPGGSLKLSCEASGFTFSRYAMSWVRHTPEKRLAWVASITFGGDPYYPDGLKG |
| | | RFTISRDNTRNILYLQMNSLRSEDTAMYYCVRHESWFAYWGQGTLVTVSAGGG |
| | | GSGGGGSGGGGSDVLMTQTPLSLPVSLGEQVSISCRSSQTIVHTDGNIYLEWY |
| | | LQNPGQSPRLLIYKISNRFSGVPDRFSGSGSGTDFTLKISRVEAEDLGIYYCF |
| | | QASHVPYTFGGGTKLEIKRTTTPAPRPPTPAPTIASQPLSLRPEACRPAAGGA |
| | | VHTRGLDFACDIYIWAPLAGTCGVLLLSLVITLYCNHRNRRRVCKCPRVDRSK |
| | | RSRLLHSDYMNMTPRRPGPTRKHYQPYAPPRDFAAYRSRVKFSRSADAPAYQQ |
| | | GQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| | | Structural formula: Nterm-(CD8 signal)-(Linker 1)- |
| | | (CD34 tag)-(Linker 2)(VH Domain)-(Linker 3)-(VL |
| | | Domain)(Linker 4)(CD8 stalk region)(CD8 |
| | | transmembrane region)(Linker 5)(CD28 cytoplasmic |
| | | region)(CD3-zeta cytoplasmic region)-Cterm |
| CAR polynucleotide | 74 | ATGGCTCTTCCGGTAACCGCTCTGCTGCTTCCTTTGGCTCTGCTTCTTCACGC |
| | | GGCTAGGCCAGGGAGCGAATTGCCTACGCAGGGGACTTTTTCAAATGTAAGCA |
| | | CGAATGTgagCTCTCCGGAAGTGAAATTGGTAGAGTCTGGTGGGGGACTGGTT |
| | | AAACCTGGAGGCAGTCTGAAGCTCTCCTGCGAAGCCAGCGGCTTTACGTTCAG |
| | | TCGGTACGCTATGTCCTGGGTGCGACACACACCAGAAAAGCGACTCGCTTGGG |
| | | TAGCGAGCATAACGTTTGGGGGCGATCCTTATTACCCGGATGGTTTGAAAGGT |
| | | CGCTTCACTATCAGTCGAGATAATACGCGAAACATTCTTTATCTTCAAATGAA |
| | | TAGCCTGCGCTCTGAAGACACCGCGATGTATTATTGTGTTAGGCATGAGTCTT |
| | | GGTTTGCATACTGGGGTCAGGGTACTCTTGTCACTGTATCTGCCGGAGGTGGG |
| | | GGTAGCGGCGGCGGAGGTAGTGGGGGAGGCGGCAGCGACGTGTTGATGACTCA |
| | | AACTCCGCTTAGCTTGCCAGTGTCTCTTGGAGAACAGGTCTCCATATCATGTC |
| | | GATCCAGTCAAACTATCGTTCATACCGATGGGAATATCTATCTCGAATGGTAT |
| | | CTGCAAAATCCAGGGCAGAGTCCCCGGCTTCTGATaTATAAGATTAGCAATAG |
| | | ATTTAGTGGTGTTCCCGACaGGTTTTCAGGTTCTGGGTCCGGAACGGATTTTA |
| | | CCCTGAAGATAAGTCGAGTTGAAGCGGAAGACCTGGGCATATACTATTGCTTC |
| | | CAGGCGTCCCACGTCCCCTATACATTTGGGGGTGGGACTAAACTGGAAATTAA |
| | | ACGtACgACGACaCCAGCCCCAAGACCCCCAACTCCCGCTCCTACCATAGCTT |
| | | CCCAACCCCTGTCACTGAGGCCAGAGGCATGCAGGCCCGCTGCGGGTGGCGCG |
| | | GTACATACGCGGGGACTGGACTTTGCATGTGACATTTACATATGGGCGCCACT |
| | | GGCGGGAACATGTGGAGTTTTGTTGCTTAGCCTGGTCATAACACTGTATTGCA |
| | | ATCATCGCAACAGACGCCGCGTCTGCAAGTGTCCCAGGGTCGACAGATCTAAG |
| | | AGAAGTAGACTTCTTCACAGTGATTATATGAATATGACGCCTCGAAGACCCGG |
| | | CCCGACACGCAAACACTATCAGCCGTATGCCCCCCCTCGGGATTTTGCTGCCT |
| | | ACCGCAGCCGCGTTAAGTTCTCTAGGTCCGCTGACGCCCCTGCCTACCAGCAG |
| | | GGTCAAAACCAACTGTACAATGAATTGAATCTTGGGAGACGGGAGGAGTATGA |
| | | CGTACTCGACAAGCGGAGGGGGAGAGATCCTGAGATGGGTGGAAAGCCTCGAC |
| | | GAAAAAACCCACAAGAAGGGTTGTATAATGAACTGCAGAAAGACAAGATGGCT |
| | | GAGGCATATAGTGAAATCGGGATGAAGGGAGAACGCCGGCGCGGGAAAGGGCA |
| | | CGATGGTCTGTATCAAGGGCTTAGTACGGCTACGAAGGACACATATGACGCTT |
| | | TGCACATGCAGGCCCTTCCTCCTAGG |
| | | Structural formula: 5′-(CD8 signal)(Linker 1)(CD34 |
| | | tag)(Linker 2)(VH Domain)(Linker 3)(VL Domain)- |
| | | (Linker 4)(CD8 stalk region)(CD8 transmembrane |
| | | region)(Linker 5)(CD28 cytoplasmic region)(CD3- |
| | | zeta cytoplasmic region)-3′ |
| CD8 signal polypeptide | 75 | MALPVTALLLPLALLLHAARP |
| CD8 signal polynucleotide | 76 | ATGGCTCTTCCGGTAACCGCTCTGCTGCTTCCTTTGGCTCTGCTTCTTCACGC |
| | | GGCTAGGCCA |
| Linker 1 polypeptide | 77 | GS |
| Linker 1 polynucleotide | 78 | GGGAGC |
| CD34 tag polypeptide | 79 | ELPTQGTFSNVSTNVS |
| CD34 tag polynucleotide | 80 | GAATTGCCTACGCAGGGGACTTTTTCAAATGTAAGCACGAATGTGAGC |
| Linker 2 polypeptide | 81 | SP |
| Linker 2 polynucleotide | 82 | TCTCCG |
| VH Domain polypeptide | 83 | EVKLVESGGGLVKPGGSLKLSCEASGFTFSRYAMSWVRHTPEKRLAWVASITF |
| | | GGDPYYPDGLKGRFTISRDNTRNILYLQMNSLRSEDTAMYYCVRHESWFAYWG |
| | | QGTLVTVSA |
| VH Domain polynucleotide | 84 | GAAGTGAAATTGGTAGAGTCTGGTGGGGGACTGGTTAAACCTGGAGGCAGTCT |
| | | GAAGCTCTCCTGCGAAGCCAGCGGCTTTACGTTCAGTCGGTACGCTATGTCCT |
| | | GGGTGCGACACACACCAGAAAAGCGACTCGCTTGGGTAGCGAGCATAACGTTT |
| | | GGGGGCGATCCTTATTACCCGGATGGTTTGAAAGGTCGCTTCACTATCAGTCG |
| | | AGATAATACGCGAAACATTCTTTATCTTCAAATGAATAGCCTGCGCTCTGAAG |
| | | ACACCGCGATGTATTATTGTGTTAGGCATGAGTCTTGGTTTGCATACTGGGGT |
| | | CAGGGTACTCTTGTCACTGTATCTGCC |
| Linker 3 polypeptide | 85 | GGGGSGGGGSGGGGS |
| Linker 3 polynucleotide | 86 | GGAGGTGGGGGTAGCGGCGGCGGAGGTAGTGGGGGAGGCGGCAGC |
| VL Domain polypeptide | 87 | DVLMTQTPLSLPVSLGEQVSISCRSSQTIVHTDGNIYLEWYLQNPGQSPRLLI |
| | | YKISNRFSGVPDRFSGSGSGTDFTLKISRVEAEDLGIYYCFQASHVPYTFGGG |
| | | TKLEIK |
| VL Domain polynucleotide | 88 | GACGTGTTGATGACTCAAACTCCGCTTAGCTTGCCAGTGTCTCTTGGAGAACA |
| | | GGTCTCCATATCATGTCGATCCAGTCAAACTATCGTTCATACCGATGGGAATA |
| | | TCTATCTCGAATGGTATCTGCAAAATCCAGGGCAGAGTCCCCGGCTTCTGATA |
| | | TATAAGATTAGCAATAGATTTAGTGGTGTTCCCGACAGGTTTTCAGGTTCTGG |
| | | GTCCGGAACGGATTTTACCCTGAAGATAAGTCGAGTTGAAGCGGAAGACCTGG |
| | | GCATATACTATTGCTTCCAGGCGTCCCACGTCCCCTATACATTTGGGGGTGGG |
| | | ACTAAACTGGAAATTAAA |
| Linker 4 polypeptide | 89 | R |
| Linker 4 polynucleotide | 90 | CGT |
| CD8 stalk polypeptide | 91 | TTTPAPRPPTPAPTIASQPLSLRPEACRPAAGGAVHTRGLDFACD |
| CD8 stalk polynucleotide | 92 | ACGACGACACCAGCCCCAAGACCCCCAACTCCCGCTCCTACCATAGCTTCCCA |
| | | ACCCCTGTCACTGAGGCCAGAGGCATGCAGGCCCGCTGCGGGTGGCGCGGTAC |
| | | ATACGCGGGGACTGGACTTTGCATGTGAC |
| CD8 transmembrane | 93 | IYIWAPLAGTCGVLLLSLVITLYCNHRNRRRVCKCPR |
| polypeptide | | |
| CD8 transmembrane | 94 | ATTTACATATGGGCGCCACTGGCGGGAACATGTGGAGTTTTGTTGCTTAGCCT |
| polynucleotide | | GGTCATAACACTGTATTGCAATCATCGCAACAGACGCCGCGTCTGCAAGTGTC |
| | | CCAGG |
| Linker 5 polypeptide | 95 | VD |
| Linker 5 polynucleotide | 96 | GTCGAC |
| CD28 cytoplasmic | 97 | RSKRSRLLHSDYMNMTPRRPGPTRKHYQPYAPPRDFAAYRS |
| polypeptide | | |
| CD28 cytoplasmic | 98 | AGATCTAAGAGAAGTAGACTTCTTCACAGTGATTATATGAATATGACGCCTCG |
| polynucleotide | | AAGACCCGGCCCGACACGCAAACACTATCAGCCGTATGCCCCCCCTCGGGATT |
| | | TTGCTGCCTACCGCAGC |
| CD3-zeta cytoplasmic | 99 | RVKFSRSADAPAYQQGQNQLYNELNLGRREEYDVLDKRRGRDPEMGGKPRRKN |
| polypeptide | | PQEGLYNELQKDKMAEAYSEIGMKGERRRGKGHDGLYQGLSTATKDTYDALHM |
| | | QALPPR |
| CD3-zeta cytoplasmic | 100 | CGCGTTAAGTTCTCTAGGTCCGCTGACGCCCCTGCCTACCAGCAGGGTCAAAA |
| polynucleotide | | CCAACTGTACAATGAATTGAATCTTGGGAGACGGGAGGAGTATGACGTACTCG |
| | | ACAAGCGGAGGGGGAGAGATCCTGAGATGGGTGGAAAGCCTCGACGAAAAAAC |
| | | CCACAAGAAGGGTTGTATAATGAACTGCAGAAAGACAAGATGGCTGAGGCATA |
| | | TAGTGAAATCGGGATGAAGGGAGAACGCCGGCGCGGGAAAGGGCACGATGGTC |
| | | TGTATCAAGGGCTTAGTACGGCTACGAAGGACACATATGACGCTTTGCACATG |
| | | CAGGCCCTTCCTCCTAGG |

Binding Molecule D

| | | |
|---|---|---|
| CAR polypeptide | 101 | MALPVTALLLPLALLLHAARPGSELPTQGTFSNVSTNVSSPEVKLVESGGGLV |
| | | KPGGSLKVSCTASGFTFSRYAMSWVRQTPERRLEWVASITFGGSAYYLDSVKG |
| | | RFTISRDNAQNILYLQMNSLVSEDTAIYNCARHQPWFDYWGQGTLVTVSAGGG |
| | | GSGGGGSGGGGSDVLLTQTPLSLPVSLGDQASISCRSSQNIXHSDGDTYLDWF |
| | | LQKPGQSPNLLIYKVSNRFSGVPDRFSGSGSGTDFTLKISRVEADDLGVYYCF |
| | | QASHVPYTFGGGTKLEVKRTTTPAPRPPTPAPTIASQPLSLRPEACRPAAGGA |
| | | VHTRGLDFACDIYIWAPLAGTCGVLLLSLVITLYCNHRNRRRVCKCPRVDRSK |
| | | RSRLLHSDYMNMTPRRPGPTRKHYQPYAPPRDFAAYRSRVKFSRSADAPAYQQ |
| | | GQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| | | Structural formula: Nterm-(CD8 signal)(Linker 1)- |
| | | (CD34 tag)(Linker 2)(VH Domain)(Linker 3)(VL |
| | | Domain)(Linker 4)(CD8 stalk region)(CD8 |
| | | transmembrane region)(Linker 5)(CD28 cytoplasmic |
| | | region)(CD3-zeta cytoplasmic region) -Cterm |
| | | X is I or V |
| CAR polynucleotide | 102 | ATGGCTCTTCCGGTAACCGCTCTGCTGCTTCCTTTGGCTCTGCTTCTTCACGC |
| | | GGCTAGGCCAGGGAGCGAATTGCCTACGCAGGGGACTTTTTCAAATGTAAGCA |
| | | CGAATGTGAGCTCTCCGGAAGTCAAATTGGTGGAGAGTGGCGGTGGGCTCGTA |
| | | AAACCAGGAGGCAGCTTGAAAGTAAGTTGCACAGCGTCTGGGTTCACTTTCAG |
| | | CCGATACGCTATGAGTTGGGTGCGGCAAACGCCCGAAAGACGATTGGAGTGGG |
| | | TAGCTAGTATTACTTTTGGGGGTTCCGCGTATTACTTGGATAGCGTTAAAGGG |
| | | AGATTCACCATATCACGAGATAATGCCCAAAACATACTCTACCTCCAGATGAA |
| | | CTCATTGGTGTCTGAAGATACTGCAATTTACAACTGCGCCAGGCACCAGCCAT |
| | | GGTTTGACTATTGGGGTCAAGGTACGTTGGTCACAGTCTCAGCCGGCGGTGGC |
| | | GGATCTGGAGGGGGAGGCAGTGGAGGAGGTGGTTCAGATGTTCTCCTGACGCA |
| | | AACTCCTTTGTCATTGCCGGTAAGTCTGGGCGACCAAGCGAGCATTTCCTGTC |
| | | GCAGCAGCCAGAACATAZTYCATAGTGATGGCGACACTTACTTGGATTGGTTC |
| | | CTCCAGAAACCGGGGCAATCTCCGAACCTCTTGATATATAAGGTCTCAAATCG |
| | | CTTTAGCGGGGTTCCCGACCGCTTTTCAGGTTCTGGCTCAGGTACCGATTTCA |
| | | CGCTGAAAATTTCTAGAGTAGAGGCTGACGATCTGGGTGTTTACTATTGTTTT |
| | | CAAGCTTCACACGTTCCTTATACGTTCGGAGGGGGGACTAAACTGGAGGTTAA |
| | | ACGTACGACGACACCAGCCCCAAGACCCCCAACTCCCGCTCCTACCATAGCTT |
| | | CCCAACCCCTGTCACTGAGGCCAGAGGCATGCAGGCCCGCTGCGGGTGGCGCG |
| | | GTACATACGCGGGGACTGGACTTTGCATGTGACATTTACATATGGGCGCCACT |
| | | GGCGGGAACATGTGGAGTTTTGTTGCTTAGCCTGGTCATAACACTGTATTGCA |
| | | ATCATCGCAACAGACGCCGCGTCTGCAAGTGTCCCAGGGTCGACAGATCTAAG |
| | | AGAAGTAGACTTCTTCACAGTGATTATATGAATATGACGCCTCGAAGACCCGG |
| | | CCCGACACGCAAACACTATCAGCCGTATGCCCCCCCTCGGGATTTTGCTGCCT |
| | | ACCGCAGCCGCGTTAAGTTCTCTAGGTCCGCTGACGCCCCTGCCTACCAGCAG |
| | | GGTCAAAACCAACTGTACAATGAATTGAATCTTGGGAGACGGGAGGAGTATGA |
| | | CGTACTCGACAAGCGGAGGGGGAGAGATCCTGAGATGGGTGGAAAGCCTCGAC |
| | | GAAAAAACCCACAAGAAGGGTTGTATAATGAACTGCAGAAAGACAAGATGGCT |
| | | GAGGCATATAGTGAAATCGGGATGAAGGGAGAACGCCGGCGCGGGAAAGGGCA |
| | | CGATGGTCTGTATCAAGGGCTTAGTACGGCTACGAAGGACACATATGACGCTT |
| | | TGCACATGCAGGCCCTTCCTCCTAGG |
| | | Structural formula: 5′-(CD8 signal)(Linker 1)(CD34 |
| | | tag)(Linker 2)(VH Domain)(Linker 3)(VL Domain)- |
| | | (Linker 4)(CD8 stalk region)(CD8 transmembrane |
| | | region)(Linker 5)(CD28 cytoplasmic region)(CD3- |
| | | zeta cytoplasmic region)-3′ |
| | | Z is A or G; Y is A or T |
| CD8 signal polypeptide | 103 | MALPVTALLLPLALLLHAARP |
| CD8 signal polynucleotide | 104 | ATGGCTCTTCCGGTAACCGCTCTGCTGCTTCCTTTGGCTCTGCTTCTTCACGC |
| | | GGCTAGGCCA |
| Linker 1 polypeptide | 105 | GS |
| Linker 1 polynucleotide | 106 | GGGAGC |
| CD34 tag polypeptide | 107 | ELPTQGTFSNVSTNVS |
| CD34 tag polynucleotide | 108 | GAATTGCCTACGCAGGGGACTTTTTCAAATGTAAGCACGAATGTGAGC |
| Linker 2 polypeptide | 109 | SP |
| Linker 2 polynucleotide | 110 | TCTCCG |
| VH Domain polypeptide | 111 | EVKLVESGGGLVKPGGSLKVSCTASGFTFSRYAMSWVRQTPERRLEWVASITF |
| | | GGSAYYLDSVKGRFTISRDNAQNILYLQMNSLVSEDTAIYNCARHQPWFDYWG |
| | | QGTLVTVSA |
| VH Domain polynucleotide | 112 | GAAGTCAAATTGGTGGAGAGTGGCGGTGGGCTCGTAAAACCAGGAGGCAGCTT |
| | | GAAAGTAAGTTGCACAGCGTCTGGGTTCACTTTCAGCCGATACGCTATGAGTT |
| | | GGGTGCGGCAAACGCCCGAAAGACGATTGGAGTGGGTAGCTAGTATTACTTTT |
| | | GGGGGTTCCGCGTATTACTTGGATAGCGTTAAAGGGAGATTCACCATATCACG |
| | | AGATAATGCCCAAAACATACTCTACCTCCAGATGAACTCATTGGTGTCTGAAG |
| | | ATACTGCAATTTACAACTGCGCCAGGCACCAGCCATGGTTTGACTATTGGGGT |
| | | CAAGGTACGTTGGTCACAGTCTCAGCC |
| Linker 3 polypeptide | 113 | GGGGSGGGGSGGGGS |
| Linker 3 polynucleotide | 114 | GGCGGTGGCGGATCTGGAGGGGGAGGCAGTGGAGGAGGTGGTTCA |
| VL Domain polypeptide | 115 | DVLLTQTPLSLPVSLGDQASISCRSSQNIXHSDGDTYLDWFLQKPGQSPNLLI |
| | | YKVSNRFSGVPDRFSGSGSGTDFTLKISRVEADDLGVYYCFQASHVPYTFGGG |
| | | TKLEVK |
| | | X is I or V |
| VL Domain polynucleotide | 116 | GATGTTCTCCTGACGCAAACTCCTTTGTCATTGCCGGTAAGTCTGGGCGACCA |
| | | AGCGAGCATTTCCTGTCGCAGCAGCCAGAACATAZTYCATAGTGATGGCGACA |
| | | CTTACTTGGATTGGTTCCTCCAGAAACCGGGGCAATCTCCGAACCTCTTGATA |
| | | TATAAGGTCTCAAATCGCTTTAGCGGGGTTCCCGACCGCTTTTCAGGTTCTGG |
| | | CTCAGGTACCGATTTCACGCTGAAAATTTCTAGAGTAGAGGCTGACGATCTGG |
| | | GTGTTTACTATTGTTTTCAAGCTTCACACGTTCCTTATACGTTCGGAGGGGGG |
| | | ACTAAACTGGAGGTTAAA |
| | | Z is A or G; Y is A or T |
| Linker 4 polypeptide | 117 | R |
| Linker 4 polynucleotide | 118 | CGT |
| CD8 stalk polypeptide | 119 | TTTPAPRPPTPAPTIASQPLSLRPEACRPAAGGAVHTRGLDFACD |
| CD8 stalk polynucleotide | 120 | ACGACGACACCAGCCCCAAGACCCCCAACTCCCGCTCCTACCATAGCTTCCCA |
| | | ACCCCTGTCACTGAGGCCAGAGGCATGCAGGCCCGCTGCGGGTGGCGCGGTAC |
| | | ATACGCGGGGACTGGACTTTGCATGTGAC |
| CD8 transmembrane | 121 | IYIWAPLAGTCGVLLLSLVITLYCNHRNRRRVCKCPR |
| polypeptide | | |
| CD8 transmembrane | 122 | ATTTACATATGGGCGCCACTGGCGGGAACATGTGGAGTTTTGTTGCTTAGCCT |
| polynucleotide | | GGTCATAACACTGTATTGCAATCATCGCAACAGACGCCGCGTCTGCAAGTGTC |
| | | CCAGG |
| Linker 5 polypeptide | 123 | VD |
| Linker 5 polynucleotide | 124 | GTCGAC |
| CD28 cytoplasmic | 125 | RSKRSRLLHSDYMNMTPRRPGPTRKHYQPYAPPRDFAAYRS |
| polypeptide | | |
| CD28 cytoplasmic | 126 | AGATCTAAGAGAAGTAGACTTCTTCACAGTGATTATATGAATATGACGCCTCG |
| polynucleotide | | AAGACCCGGCCCGACACGCAAACACTATCAGCCGTATGCCCCCCCTCGGGATT |
| | | TTGCTGCCTACCGCAGC |
| CD3-zeta cytoplasmic | 127 | RVKFSRSADAPAYQQGQNQLYNELNLGRREEYDVLDKRRGRDPEMGGKPRRKN |
| polypeptide | | PQEGLYNELQKDKMAEAYSEIGMKGERRRGKGHDGLYQGLSTATKDTYDALHM |
| | | QALPPR |
| CD3-zeta cytoplasmic | 128 | CGCGTTAAGTTCTCTAGGTCCGCTGACGCCCCTGCCTACCAGCAGGGTCAAAA |
| polynucleotide | | CCAACTGTACAATGAATTGAATCTTGGGAGACGGGAGGAGTATGACGTACTCG |
| | | ACAAGCGGAGGGGGAGAGATCCTGAGATGGGTGGAAAGCCTCGACGAAAAAAC |
| | | CCACAAGAAGGGTTGTATAATGAACTGCAGAAAGACAAGATGGCTGAGGCATA |
| | | TAGTGAAATCGGGATGAAGGGAGAACGCCGGCGCGGGAAAGGGCACGATGGTC |
| | | TGTATCAAGGGCTTAGTACGGCTACGAAGGACACATATGACGCTTTGCACATG |
| | | CAGGCCCTTCCTCCTAGG |
| Mesothelin isoform 2 | 129 | MALPTARPLLGSCGTPALGSLLFLLFSLGWVQPSRTLAGETGQEAAPLDGVLA |
| polypeptide (unique | | NPPNISSLSPRQLLGFPCAEVSGLSTERVRELAVALAQKNVKLSTEQLRCLAH |
| sequence in bold text) | | RLSEPPEDLDALPLDLLLFLNPDAFSGPQACTRFFSRITKANVDLLPRGAPER |
| | | QRLLPAALACWGVRGSLLSEADVRALGGLACDLPGRFVAESAEVLLPRLVSCP |
| | | GPLDQDQQEAARAALQGGGPPYGPPSTWSVSTMDALRGLLPVLGQPIIRSIPQ |
| | | GIVAAWRQRSSRDPSWRQPERTILRPRFRREVEKTACPSGKKAREIDESLIFY |
| | | KKWELEACVDAALLATQMDRVNAIPFTYEQLDVLKHKLDELYPQGYPESVIQH |
| | | LGYLFLKMSPEDIRKWNVTSLETLKALLEVNKGHEMSPQAPRRPLPQVATLID |
| | | RFVKGRGQLDKDTLDTLTAFYPGYLCSLSPEELSSVPPSSIWAVRPQDLDTCD |
| | | PRQLDVLYPKARLAFQNMNGSEYFVKIQSFLGGAPTEDLKALSQQNVSMDLAT |
| | | FMKLRTDAVLPLTVAEVQKLLGPHVEGLKAEERHRPVRDWILRQRQDDLDTLG |
| | | LGLQGGIPNGYLVLDLSMQEALSGTPCLLGPGPVLTVLALLLASTLA |
| Mesothelin canonical | 130 | MALPTARPLLGSCGTPALGSLLFLLFSLGWVQPSRTLAGETGQEAAPLDGVLA |
| polypeptide | | NPPNISSLSPRQLLGFPCAEVSGLSTERVRELAVALAQKNVKLSTEQLRCLAH |
| | | RLSEPPEDLDALPLDLLLFLNPDAFSGPQACTRFFSRITKANVDLLPRGAPER |
| | | QRLLPAALACWGVRGSLLSEADVRALGGLACDLPGRFVAESAEVLLPRLVSCP |
| | | GPLDQDQQEAARAALQGGGPPYGPPSTWSVSTMDALRGLLPVLGQPIIRSIPQ |
| | | GIVAAWRQRSSRDPSWRQPERTILRPRFRREVEKTACPSGKKAREIDESLIFY |
| | | KKWELEACVDAALLATQMDRVNAIPFTYEQLDVLKHKLDELYPQGYPESVIQH |
| | | LGYLFLKMSPEDIRKWNVTSLETLKALLEVNKGHEMSPQVATLIDRFVKGRGQ |
| | | LDKDTLDTLTAFYPGYLCSLSPEELSSVPPSSIWAVRPQDLDTCDPRQLDVLY |
| | | PKARLAFQNMNGSEYFVKIQSFLGGAPTEDLKALSQQNVSMDLATFMKLRTDA |
| | | VLPLTVAEVQKLLGPHVEGLKAEERHRPVRDWILRQRQDDLDTLGLGLQGGIP |
| | | NGYLVLDLSMQEALSGTPCLLGPGPVLTVLALLLASTLA |
| Mesothelin isoform 2 | 131 | PQAPRRPL |
| polypeptide subsequence | | |
| Mesothelin isoform 2 | 132 | RPLPQVATLIDR |
| polypeptide subsequence | | |
| Mesothelin canonical | 133 | GHEMSPQVATLIDR |
| polypeptide subsequence | | |

Chimeric PD1 Molecule A

| | | |
|---|---|---|
| PD1 membrane signal | 134 | ATGCAGATCCCCCAGGCCCCCTGGCCCGTGGTGTGGGCCGTGCTGCAGCTGGG |
| polynucleotide | | CTGGAGGCCCGGCTGG |
| PD1 membrane signal | 135 | MQIPQAPWPVVWAVLQLGWRPGW |
| polypeptide | | |
| PD1 region polynucleotide | 136 | TTCCTGGACAGCCCCGACAGGCCCTGGAACCCCCCCACCTTCAGCCCCGCCCT |
| | | GCTGGTGGTGACCGAGGGCGACAACGCCACCTTCACCTGCAGCTTCAGCAACA |
| | | CCAGCGAGAGCTTCGTGCTGAACTGGTACAGGATGAGCCCCAGCAACCAGACC |
| | | GACAAGCTGGCCGCCTTCCCCGAGGACAGGAGCCAGCCCGGCCAGGACTGCAG |
| | | GTTCAGGGTGACCCAGCTGCCCAACGGCAGGGACTTCCACATGAGCGTGGTGA |
| | | GGGCCAGGAGGAACGACAGCGGCACCTACCTGTGCGGCGCCATCAGCCTGGCC |
| | | CCCAAGGCCCAGATCAAGGAGAGCCTGAGGGCCGAGCTGAGGGTGACCGAGAG |
| | | GAGGGCCGAGGTGCCCACCGCCCACCCCAGCCCCAGCCCCAGGCCCGCCGGCC |
| | | AGTTCCAGACCCTGGTG |
| PD1 region polypeptide | 137 | FLDSPDRPWNPPTFSPALLVVTEGDNATFTCSFSNTSESFVLNWYRMSPSNQT |
| | | DKLAAFPEDRSQPGQDCRFRVTQLPNGRDFHMSVVRARRNDSGTYLCGAISLA |
| | | PKAQIKESLRAELRVTERRAEVPTAHPSPSPRPAGQFQTLV |
| truncated CD28 | 138 | TGCCCCAGCCCCCTGTTCCCCGGCCCCAGCAAGCCC |
| region polynucleotide | | |
| truncated CD28 | 139 | CPSPLFPGPSKP |
| region polypeptide | | |
| CD28 transmembrane | 140 | TTCTGGGTGCTGGTGGTGGTGGGCGGCGTGCTGGCCTGCTACAGCCTGCTGGT |
| region polynucleotide | | GACCGTGGCCTTCATCATCTTCTGGGTG |
| CD28 transmembrane | 141 | FWVLVVVGGVLACYSLLVTVAFIIFWV |
| region polypeptide | | |
| DAP10 region | 142 | CTGTGCGCCAGGCCCAGGAGGAGCCCCGCCCAGGAGGACGGCAAGGTGTACAT |
| polynucleotide | | CAACATGCCCGGCAGGGGC |
| DAP10 region polypeptide | 143 | LCARPRRSPAQEDGKVYINMPGRG |
| CD3z region | 144 | AGGGTGAAGTTCAGCAGGAGCGCCGACGCCCCCGCCTACCAGCAGGGCCAGAA |
| polynucleotide | | CCAGCTGTACAACGAGCTGAACCTGGGCAGGAGGGAGGAGTACGACGTGCTGG |
| | | ACAAGAGGAGGGGCAGGGACCCCGAGATGGGCGGCAAGCCCCAGAGGAGGAAG |
| | | AACCCCCAGGAGGGCCTGTACAACGAGCTGCAGAAGGACAAGATGGCCGAGGC |
| | | CTACAGCGAGATCGGCATGAAGGGCGAGAGGAGGAGGGGCAAGGGCCACGACG |
| | | GCCTGTACCAGGGCCTGAGCACCGCCACCAAGGACACCTACGACGCCCTGCAC |
| | | ATGCAGGCCCTGCCCCCCAGG |
| CD3z region polypeptide | 145 | RVKFSRSADAPAYQQGQNQLYNELNLGRREEYDVLDKRRGRDPEMGGKPQRRK |
| | | NPQEGLYNELQKDKMAEAYSEIGMKGERRRGKGHDGLYQGLSTATKDTYDALH |
| | | MQALPPR |
| chimeric PD1-A | 146 | ATGCAGATCCCCCAGGCCCCCTGGCCCGTGGTGTGGGCCGTGCTGCAGCTGGG |
| polynucleotide | | CTGGAGGCCCGGCTGGTTCCTGGACAGCCCCGACAGGCCCTGGAACCCCCCCA |
| | | CCTTCAGCCCCGCCCTGCTGGTGGTGACCGAGGGCGACAACGCCACCTTCACC |
| | | TGCAGCTTCAGCAACACCAGCGAGAGCTTCGTGCTGAACTGGTACAGGATGAG |
| | | CCCCAGCAACCAGACCGACAAGCTGGCCGCCTTCCCCGAGGACAGGAGCCAGC |
| | | CCGGCCAGGACTGCAGGTTCAGGGTGACCCAGCTGCCCAACGGCAGGGACTTC |
| | | CACATGAGCGTGGTGAGGGCCAGGAGGAACGACAGCGGCACCTACCTGTGCGG |
| | | CGCCATCAGCCTGGCCCCCAAGGCCCAGATCAAGGAGAGCCTGAGGGCCGAGC |
| | | TGAGGGTGACCGAGAGGAGGGCCGAGGTGCCCACCGCCCACCCCAGCCCCAGC |
| | | CCCAGGCCCGCCGGCCAGTTCCAGACCCTGGTGTGCCCCAGCCCCCTGTTCCC |
| | | CGGCCCCAGCAAGCCCTTCTGGGTGCTGGTGGTGGTGGGCGGCGTGCTGGCCT |
| | | GCTACAGCCTGCTGGTGACCGTGGCCTTCATCATCTTCTGGGTGCTGTGCGCC |
| | | AGGCCCAGGAGGAGCCCCGCCCAGGAGGACGGCAAGGTGTACATCAACATGCC |
| | | CGGCAGGGGCAGGGTGAAGTTCAGCAGGAGCGCCGACGCCCCCGCCTACCAGC |
| | | AGGGCCAGAACCAGCTGTACAACGAGCTGAACCTGGGCAGGAGGGAGGAGTAC |
| | | GACGTGCTGGACAAGAGGAGGGGCAGGGACCCCGAGATGGGCGGCAAGCCCCA |
| | | GAGGAGGAAGAACCCCCAGGAGGGCCTGTACAACGAGCTGCAGAAGGACAAGA |
| | | TGGCCGAGGCCTACAGCGAGATCGGCATGAAGGGCGAGAGGAGGAGGGGCAAG |
| | | GGCCACGACGGCCTGTACCAGGGCCTGAGCACCGCCACCAAGGACACCTACGA |
| | | CGCCCTGCACATGCAGGCCCTGCCCCCCAGG |
| | | Structural formula: 5′-(PD1 signal)(PD1 region |
| | | (extracellular))(truncated CD28 region |
| | | (extracellular))(CD28 transmembrane region)(DAP10 |
| | | region (cytoplasmic))(CD3-zeta region |
| | | (cytoplasmic))-3′ |
| chimeric PD1-A | 147 | MQIPQAPWPVVWAVLQLGWRPGWFLDSPDRPWNPPTFSPALLVVTEGDNATFT |
| polypeptide | | CSFSNTSESFVLNWYRMSPSNQTDKLAAFPEDRSQPGQDCRFRVTQLPNGRDF |
| | | HMSVVRARRNDSGTYLCGAISLAPKAQIKESLRAELRVTERRAEVPTAHPSPS |
| | | PRPAGQFQTLVCPSPLFPGPSKPFWVLVVVGGVLACYSLLVTVAFIIFWVLCA |
| | | RPRRSPAQEDGKVYINMPGRGRVKFSRSADAPAYQQGQNQLYNELNLGRREEY |
| | | DVLDKRRGRDPEMGGKPQRRKNPQEGLYNELQKDKMAEAYSEIGMKGERRRGK |
| | | GHDGLYQGLSTATKDTYDALHMQALPPR |
| | | Structural formula: Nterm-(PD1 signal)(PD1 region |
| | | (extracellular))(truncated CD28 region |
| | | (extracellular))(CD28 transmembrane region)(DAP10 |
| | | region (cytoplasmic))(CD3-zeta region |
| | | (cytoplasmic))-Cterm |

Chimeric PD1 Molecule B

| | | |
|---|---|---|
| CD8 signal polynucleotide | 148 | ATGGCTCTTCCGGTAACCGCTCTGCTGCTTCCTTTGGCTCTGCTTCTTCACGC |
| | | GGCTAGGCCA |
| CD8 signal polypeptide | 149 | MALPVTALLLPLALLLHAARP |
| Linker 1 polynucleotide | 150 | GGGAGC |
| Linker 1 polypeptide | 151 | GS |
| CD34 tag polynucleotide | 152 | GAATTGCCTACGCAGGGGACTTTTTCAAATGTAAGCACGAATGTGAGC |
| CD34 tag polypeptide | 153 | ELPTQGTFSNVSTNVS |
| Linker 2 polynucleotide | 154 | TCTCCGGGATCCCCAGGATGG |
| Linker 2 polypeptide | 155 | SPGSPGW |
| PD1 region polynucleotide | 156 | TTCCTGGACAGCCCAGATCGGCCCTGGAATCCCCCTACCTTTTCCCCTGCCCT |
| | | GCTGGTGGTGACAGAGGGCGACAACGCCACCTTCACATGCAGCTTTTCCAACA |
| | | CCTCTGAGAGCTTCGTGCTGAATTGGTACAGAATGTCCCCATCTAACCAGACA |
| | | GATAAGCTGGCCGCATTTCCAGAGGACAGGTCCCAGCCTGGACAGGATTGCCG |
| | | CTTCCGGGTGACCCAGCTGCCCAATGGCAGAGACTTTCACATGAGCGTGGTGA |
| | | GGGCCCGGAGAAACGATTCCGGCACATACCTGTGCGGAGCCATCTCTCTGGCC |
| | | CCAAAGGCACAGATCAAGGAGAGCCTGAGGGCAGAGCTGAGGGTGACCGAGAG |
| | | GAGGGCAGAGGTGCCTACAGCACACCCAAGCCCTTCCCCAAGACCAGCAGGAC |
| | | AGTTCCAGACCCTGGTG |
| PD1 region polypeptide | 157 | FLDSPDRPWNPPTFSPALLVVTEGDNATFTCSESNTSESFVLNWYRMSPSNQT |
| | | DKLAAFPEDRSQPGQDCRFRVTQLPNGRDFHMSVVRARRNDSGTYLCGAISLA |
| | | PKAQIKESLRAELRVTERRAEVPTAHPSPSPRPAGQFQTLV |
| truncated CD28 | 158 | TGCCCTTCCCCACTGTTCCCAGGACCTTCTAAGCCC |
| region polynucleotide | | |
| truncated CD28 | 159 | FLDSPDRPWNPPTFSPALLVVTEGDNATFTCSFSNTSESFVLNWYRMSPSNQT |
| region polypeptide | | DKLAAFPEDRSQPGQDCRFRVTQLPNGRDFHMSVVRARRNDSGTYLCGAISLA |
| | | PKAQIKESLRAELRVTERRAEVPTAHPSPSPRPAGQFQTLV |
| CD28 transmembrane | 160 | TTTTGGGTGCTGGTGGTGGTGGGAGGCGTGCTGGCCTGCTACTCTCTGCTGGT |
| region polynucleotide | | GACAGTGGCCTTCATCATCTTTTGGGTG |
| CD28 transmembrane | 161 | FWVLVVVGGVLACYSLLVTVAFIIFWV |
| region polypeptide | | |
| DAP10 region | 162 | CTGTGCGCCCGCCCTCGGAGAAGCCCAGCACAGGAGGACGGCAAGGTGTATAT |
| polynucleotide | | CAATATGCCAGGCAGAGGC |
| DAP10 region polypeptide | 163 | LCARPRRSPAQEDGKVYINMPGRG |
| CD3z region | 164 | AGGGTGAAGTTTTCTCGGAGCGCCGATGCACCAGCATACCAGCAGGGACAGAA |
| polynucleotide | | CCAGCTGTATAACGAGCTGAATCTGGGCAGGCGCGAGGAGTACGACGTGCTGG |
| | | ATAAGCGGAGAGGCAGAGACCCAGAGATGGGAGGCAAGCCACAGAGGAGGAAG |
| | | AACCCTCAGGAGGGCCTGTACAATGAGCTGCAGAAGGACAAGATGGCCGAGGC |
| | | CTATTCCGAGATCGGCATGAAGGGAGAGCGGAGAAGGGGCAAGGGACACGATG |
| | | GCCTGTACCAGGGCCTGTCTACCGCCACAAAGGACACCTATGATGCCCTGCAC |
| | | ATGCAGGCCCTGCCACCCAGG |
| CD3z region polypeptide | 165 | RVKFSRSADAPAYQQGQNQLYNELNLGRREEYDVLDKRRGRDPEMGGKPQRRK |
| | | NPQEGLYNELQKDKMAEAYSEIGMKGERRRGKGHDGLYQGLSTATKDTYDALH |
| | | MQALPPR |
| chimeric PD1-B | 166 | ATGGCTCTTCCGGTAACCGCTCTGCTGCTTCCTTTGGCTCTGCTTCTTCACGC |
| polynucleotide | | GGCTAGGCCAGGGAGCGAATTGCCTACGCAGGGGACTTTTTCAAATGTAAGCA |
| | | CGAATGTgagCTCTCCGGGATCCCCAGGATGGTTCCTGGACAGCCCAGATCGG |
| | | CCCTGGAATCCCCCTACCTTTTCCCCTGCCCTGCTGGTGGTGACAGAGGGCGA |
| | | CAACGCCACCTTCACATGCAGCTTTTCCAACACCTCTGAGAGCTTCGTGCTGA |
| | | ATTGGTACAGAATGTCCCCATCTAACCAGACAGATAAGCTGGCCGCATTTCCA |
| | | GAGGACAGGTCCCAGCCTGGACAGGATTGCCGCTTCCGGGTGACCCAGCTGCC |
| | | CAATGGCAGAGACTTTCACATGAGCGTGGTGAGGGCCCGGAGAAACGATTCCG |
| | | GCACATACCTGTGCGGAGCCATCTCTCTGGCCCCAAAGGCACAGATCAAGGAG |
| | | AGCCTGAGGGCAGAGCTGAGGGTGACCGAGAGGAGGGCAGAGGTGCCTACAGC |
| | | ACACCCAAGCCCTTCCCCAAGACCAGCAGGACAGTTCCAGACCCTGGTGTGCC |
| | | CTTCCCCACTGTTCCCAGGACCTTCTAAGCCCTTTTGGGTGCTGGTGGTGGTG |
| | | GGAGGCGTGCTGGCCTGCTACTCTCTGCTGGTGACAGTGGCCTTCATCATCTT |
| | | TTGGGTGCTGTGCGCCCGCCCTCGGAGAAGCCCAGCACAGGAGGACGGCAAGG |
| | | TGTATATCAATATGCCAGGCAGAGGCAGGGTGAAGTTTTCTCGGAGCGCCGAT |
| | | GCACCAGCATACCAGCAGGGACAGAACCAGCTGTATAACGAGCTGAATCTGGG |
| | | CAGGCGCGAGGAGTACGACGTGCTGGATAAGCGGAGAGGCAGAGACCCAGAGA |
| | | TGGGAGGCAAGCCACAGAGGAGGAAGAACCCTCAGGAGGGCCTGTACAATGAG |
| | | CTGCAGAAGGACAAGATGGCCGAGGCCTATTCCGAGATCGGCATGAAGGGAGA |
| | | GCGGAGAAGGGGCAAGGGACACGATGGCCTGTACCAGGGCCTGTCTACCGCCA |
| | | CAAAGGACACCTATGATGCCCTGCACATGCAGGCCCTGCCACCCAGG |
| | | Structural formula: 5′-(CD8 signal)(linker 1)(CD34 |
| | | tag)(linker 2)(PD1 region (extracellular))- |
| | | (truncated CD28 region (extracellular))(CD28 |
| | | transmembrane region)(DAP10 region (cytoplasmic))- |
| | | (CD3-zeta region (cytoplasmic))-3′ |
| chimeric PD1-B | 167 | MALPVTALLLPLALLLHAARPGSELPTQGTFSNVSTNVSSPGSPGWELDSPDR |
| polypeptide | | PWNPPTFSPALLVVTEGDNATFTCSFSNTSESFVLNWYRMSPSNQTDKLAAFP |
| | | EDRSQPGQDCRFRVTQLPNGRDFHMSVVRARRNDSGTYLCGAISLAPKAQIKE |
| | | SLRAELRVTERRAEVPTAHPSPSPRPAGQFQTLVCPSPLFPGPSKPFWVLVVV |
| | | GGVLACYSLLVTVAFIIFWVLCARPRRSPAQEDGKVYINMPGRGRVKFSRSAD |
| | | APAYQQGQNQLYNELNLGRREEYDVLDKRRGRDPEMGGKPQRRKNPQEGLYNE |
| | | LQKDKMAEAYSEIGMKGERRRGKGHDGLYQGLSTATKDTYDALHMQALPPR |
| | | Nterm-(CD8 signal)(linker 1)(CD34 tag)(linker 2)- |
| | | (PD1 region (extracellular))(truncated CD28 region |
| | | (extracellular)(CD28 transmembrane region)(DAP10 |
| | | region (cytoplasmic))(CD3-zeta region |
| | | (cytoplasmic))-Cterm |

Binding Molecule E

| | | |
|---|---|---|
| CAR polypeptide | 168 | MALPVTALLLPLALLLHAARPGSELPTQGTFSNVSTNVSSPEVKLVESGGGLV |
| | | KPGGSLKVSCTASGFTFSRYAMSWVRQTPERRLEWVASITFGGSAYYLDSVKG |
| | | RFTISRDNAQNILYLQMNSLVSEDTAIYNCARHQPWFDYWGQGTLVTVSAGGG |
| | | GSGGGGSGGGGSDVLLTQTPLSLPVSLGDQASISCRSSQNIVHSDGDTYLDWF |
| | | LQKPGQSPNLLIYKVSNRFSGVPDRFSGSGSGTDFTLKISRVEADDLGVYYCF |
| | | QASHVPYTFGGGTKLEVKRTTTPAPRPPTPAPTIASQPLSLRPEACRPAAGGA |
| | | VHTRGLDFACDIYIWAPLAGTCGVLLLSLVITLYCNHRNRRRVCKCPRVDRSK |
| | | RSRLLHSDYMNMTPRRPGPTRKHYQPYAPPRDFAAYRSRVKFSRSADAPAYQQ |
| | | GQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| | | Structural formula: Nterm-(CD8 signal)(Linker 1)- |
| | | (CD34 tag)(Linker 2)(VH Domain)(Linker 3)(VL |
| | | Domain)(Linker 4)(CD8 stalk region)(CD8 |
| | | transmembrane region)(Linker 5)(CD28 cytoplasmic |
| | | region)(CD3-zeta cytoplasmic region)-Cterm |
| CAR polynucleotide | 169 | ATGGCTCTTCCGGTAACCGCTCTGCTGCTTCCTTTGGCTCTGCTTCTTCACGC |
| | | GGCTAGGCCAGGGAGCGAATTGCCTACGCAGGGGACTTTTTCAAATGTAAGCA |
| | | CGAATGTGAGCTCTCCGGAAGTCAAATTGGTGGAGAGTGGCGGTGGGCTCGTA |
| | | AAACCAGGAGGCAGCTTGAAAGTAAGTTGCACAGCGTCTGGGTTCACTTTCAG |
| | | CCGATACGCTATGAGTTGGGTGCGGCAAACGCCCGAAAGACGATTGGAGTGGG |
| | | TAGCTAGTATTACTTTTGGGGGTTCCGCGTATTACTTGGATAGCGTTAAAGGG |
| | | AGATTCACCATATCACGAGATAATGCCCAAAACATACTCTACCTCCAGATGAA |
| | | CTCATTGGTGTCTGAAGATACTGCAATTTACAACTGCGCCAGGCACCAGCCAT |
| | | GGTTTGACTATTGGGGTCAAGGTACGTTGGTCACAGTCTCAGCCGGCGGTGGC |
| | | GGATCTGGAGGGGGAGGCAGTGGAGGAGGTGGTTCAGATGTTCTCCTGACGCA |
| | | AACTCCTTTGTCATTGCCGGTAAGTCTGGGCGACCAAGCGAGCATTTCCTGTC |
| | | GCAGCAGCCAGAACATAGTTCATAGTGATGGCGACACTTACTTGGATTGGTTC |
| | | CTCCAGAAACCGGGGCAATCTCCGAACCTCTTGATATATAAGGTCTCAAATCG |
| | | CTTTAGCGGGGTTCCCGACCGCTTTTCAGGTTCTGGCTCAGGTACCGATTTCA |
| | | CGCTGAAAATTTCTAGAGTAGAGGCTGACGATCTGGGTGTTTACTATTGTTTT |
| | | CAAGCTTCACACGTTCCTTATACGTTCGGAGGGGGGACTAAACTGGAGGTTAA |
| | | ACGTACGACGACACCAGCCCCAAGACCCCCAACTCCCGCTCCTACCATAGCTT |
| | | CCCAACCCCTGTCACTGAGGCCAGAGGCATGCAGGCCCGCTGCGGGTGGCGCG |
| | | GTACATACGCGGGGACTGGACTTTGCATGTGACATTTACATATGGGCGCCACT |
| | | GGCGGGAACATGTGGAGTTTTGTTGCTTAGCCTGGTCATAACACTGTATTGCA |
| | | ATCATCGCAACAGACGCCGCGTCTGCAAGTGTCCCAGGGTCGACAGATCTAAG |
| | | AGAAGTAGACTTCTTCACAGTGATTATATGAATATGACGCCTCGAAGACCCGG |
| | | CCCGACACGCAAACACTATCAGCCGTATGCCCCCCCTCGGGATTTTGCTGCCT |
| | | ACCGCAGCCGCGTTAAGTTCTCTAGGTCCGCTGACGCCCCTGCCTACCAGCAG |
| | | GGTCAAAACCAACTGTACAATGAATTGAATCTTGGGAGACGGGAGGAGTATGA |
| | | CGTACTCGACAAGCGGAGGGGGAGAGATCCTGAGATGGGTGGAAAGCCTCGAC |
| | | GAAAAAACCCACAAGAAGGGTTGTATAATGAACTGCAGAAAGACAAGATGGCT |
| | | GAGGCATATAGTGAAATCGGGATGAAGGGAGAACGCCGGCGCGGGAAAGGGCA |
| | | CGATGGTCTGTATCAAGGGCTTAGTACGGCTACGAAGGACACATATGACGCTT |
| | | TGCACATGCAGGCCCTTCCTCCTAGG |
| | | Structural formula: 5′-(CD8 signal)(Linker 1)(CD34 |
| | | tag)(Linker 2)(VH Domain)(Linker 3)(VL Domain)- |
| | | (Linker 4)(CD8 stalk region)(CD8 transmembrane |
| | | region)(Linker 5)(CD28 cytoplasmic region)(CD3- |
| | | zeta cytoplasmic region)-3′ |
| CD8 signal polypeptide | 170 | MALPVTALLLPLALLLHAARP |
| CD8 signal polynucleotide | 171 | ATGGCTCTTCCGGTAACCGCTCTGCTGCTTCCTTTGGCTCTGCTTCTTCACGC |
| | | GGCTAGGCCA |
| Linker 1 polypeptide | 172 | GS |
| Linker 1 polynucleotide | 173 | GGGAGC |
| CD34 tag polypeptide | 174 | ELPTQGTFSNVSTNVS |
| CD34 tag polynucleotide | 175 | GAATTGCCTACGCAGGGGACTTTTTCAAATGTAAGCACGAATGTGAGC |
| Linker 2 polypeptide | 176 | SP |
| Linker 2 polynucleotide | 177 | TCTCCG |
| VH Domain polypeptide | 178 | EVKLVESGGGLVKPGGSLKVSCTASGFTFSRYAMSWVRQTPERRLEWVASITF |
| | | GGSAYYLDSVKGRFTISRDNAQNILYLQMNSLVSEDTAIYNCARHQPWFDYWG |
| | | QGTLVTVSA |
| VH Domain polynucleotide | 179 | GAAGTCAAATTGGTGGAGAGTGGCGGTGGGCTCGTAAAACCAGGAGGCAGCTT |
| | | GAAAGTAAGTTGCACAGCGTCTGGGTTCACTTTCAGCCGATACGCTATGAGTT |
| | | GGGTGCGGCAAACGCCCGAAAGACGATTGGAGTGGGTAGCTAGTATTACTTTT |
| | | GGGGGTTCCGCGTATTACTTGGATAGCGTTAAAGGGAGATTCACCATATCACG |
| | | AGATAATGCCCAAAACATACTCTACCTCCAGATGAACTCATTGGTGTCTGAAG |
| | | ATACTGCAATTTACAACTGCGCCAGGCACCAGCCATGGTTTGACTATTGGGGT |
| | | CAAGGTACGTTGGTCACAGTCTCAGCC |
| Linker 3 polypeptide | 180 | GGGGSGGGGSGGGGS |
| Linker 3 polynucleotide | 181 | GGCGGTGGCGGATCTGGAGGGGGAGGCAGTGGAGGAGGTGGTTCA |
| VL Domain polypeptide | 182 | DVLLTQTPLSLPVSLGDQASISCRSSQNIVHSDGDTYLDWFLQKPGQSPNLLI |
| | | YKVSNRFSGVPDRFSGSGSGTDFTLKISRVEADDLGVYYCFQASHVPYTFGGG |
| | | TKLEVK |
| VL Domain polynucleotide | 183 | GATGTTCTCCTGACGCAAACTCCTTTGTCATTGCCGGTAAGTCTGGGCGACCA |
| | | AGCGAGCATTTCCTGTCGCAGCAGCCAGAACATAGTTCATAGTGATGGCGACA |
| | | CTTACTTGGATTGGTTCCTCCAGAAACCGGGGCAATCTCCGAACCTCTTGATA |
| | | TATAAGGTCTCAAATCGCTTTAGCGGGGTTCCCGACCGCTTTTCAGGTTCTGG |
| | | CTCAGGTACCGATTTCACGCTGAAAATTTCTAGAGTAGAGGCTGACGATCTGG |
| | | GTGTTTACTATTGTTTTCAAGCTTCACACGTTCCTTATACGTTCGGAGGGGGG |
| | | ACTAAACTGGAGGTTAAA |
| Linker 4 polypeptide | 184 | R |
| Linker 4 polynucleotide | 185 | CGT |
| CD8 stalk polypeptide | 186 | TTTPAPRPPTPAPTIASQPLSLRPEACRPAAGGAVHTRGLDFACD |
| CD8 stalk polynucleotide | 187 | ACGACGACACCAGCCCCAAGACCCCCAACTCCCGCTCCTACCATAGCTTCCCA |
| | | ACCCCTGTCACTGAGGCCAGAGGCATGCAGGCCCGCTGCGGGTGGCGCGGTAC |
| | | ATACGCGGGGACTGGACTTTGCATGTGAC |
| CD8 transmembrane | 188 | IYIWAPLAGTCGVLLLSLVITLYCNHRNRRRVCKCPR |
| polypeptide | | |
| CD8 transmembrane | 189 | ATTTACATATGGGCGCCACTGGCGGGAACATGTGGAGTTTTGTTGCTTAGCCT |
| polynucleotide | | GGTCATAACACTGTATTGCAATCATCGCAACAGACGCCGCGTCTGCAAGTGTC |
| | | CCAGG |
| Linker 5 polypeptide | 190 | VD |
| Linker 5 polynucleotide | 191 | GTCGAC |
| CD28 cytoplasmic | 192 | RSKRSRLLHSDYMNMTPRRPGPTRKHYQPYAPPRDFAAYRS |
| polypeptide | | |
| CD28 cytoplasmic | 193 | AGATCTAAGAGAAGTAGACTTCTTCACAGTGATTATATGAATATGACGCCTCG |
| polynucleotide | | AAGACCCGGCCCGACACGCAAACACTATCAGCCGTATGCCCCCCCTCGGGATT |
| | | TTGCTGCCTACCGCAGC |
| CD3-zeta cytoplasmic | 194 | RVKFSRSADAPAYQQGQNQLYNELNLGRREEYDVLDKRRGRDPEMGGKPRRKN |
| polypeptide | | PQEGLYNELQKDKMAEAYSEIGMKGERRRGKGHDGLYQGLSTATKDTYDALHM |
| | | QALPPR |
| CD3-zeta cytoplasmic | 195 | CGCGTTAAGTTCTCTAGGTCCGCTGACGCCCCTGCCTACCAGCAGGGTCAAAA |
| polynucleotide | | CCAACTGTACAATGAATTGAATCTTGGGAGACGGGAGGAGTATGACGTACTCG |
| | | ACAAGCGGAGGGGGAGAGATCCTGAGATGGGTGGAAAGCCTCGACGAAAAAAC |
| | | CCACAAGAAGGGTTGTATAATGAACTGCAGAAAGACAAGATGGCTGAGGCATA |
| | | TAGTGAAATCGGGATGAAGGGAGAACGCCGGCGCGGGAAAGGGCACGATGGTC |
| | | TGTATCAAGGGCTTAGTACGGCTACGAAGGACACATATGACGCTTTGCACATG |
| | | CAGGCCCTTCCTCCTAGG |
| Binding Molecule C | 196 | GSELPTQGTFSNVSTNVSSPEVKLVESGGGLVKPGGSLKLSCEASGFTFSRYA |
| CAR polypeptide (without | | MSWVRHTPEKRLAWVASITFGGDPYYPDGLKGRFTISRDNTRNILYLQMNSLR |
| signal region) | | SEDTAMYYCVRHESWFAYWGQGTLVTVSAGGGGSGGGGSGGGGSDVLMTQTPL |
| | | SLPVSLGEQVSISCRSSQTIVHTDGNIYLEWYLQNPGQSPRLLIYKISNRFSG |
| | | VPDRFSGSGSGTDFTLKISRVEAEDLGIYYCFQASHVPYTFGGGTKLEIKRTT |
| | | TPAPRPPTPAPTIASQPLSLRPEACRPAAGGAVHTRGLDFACDIYIWAPLAGT |
| | | CGVLLLSLVITLYCNHRNRRRVCKCPRVDRSKRSRLLHSDYMNMTPRRPGPTR |
| | | KHYQPYAPPRDFAAYRSRVKESRSADAPAYQQGQNQLYNELNLGRREEYDVLD |
| | | KRRGRDPEMGGKPRRKNPQEGLYNELQKDKMAEAYSEIGMKGERRRGKGHDGL |
| | | YQGLSTATKDTYDALHMQALPPR |
| | | Structural formula: Nterm-(Linker 1)(CD34 tag)- |
| | | (Linker 2)(VH Domain)(Linker 3)(VL Domain)(Linker |
| | | 4)(CD8 stalk region)(CD8 transmembrane region)- |
| | | (Linker 5)(CD28 cytoplasmic region)(CD3-zeta |
| | | cytoplasmic region)-Cterm |
| Binding Molecule D | 197 | GSELPTQGTFSNVSTNVSSPEVKLVESGGGLVKPGGSLKVSCTASGFTFSRYA |
| CAR polypeptide (without | | MSWVRQTPERRLEWVASITFGGSAYYLDSVKGRFTISRDNAQNILYLQMNSLV |
| signal region) | | SEDTAIYNCARHQPWFDYWGQGTLVTVSAGGGGSGGGGSGGGGSDVLLTQTPL |
| | | SLPVSLGDQASISCRSSQNIXHSDGDTYLDWFLQKPGQSPNLLIYKVSNRFSG |
| | | VPDRFSGSGSGTDFTLKISRVEADDLGVYYCFQASHVPYTFGGGTKLEVKRTT |
| | | TPAPRPPTPAPTIASQPLSLRPEACRPAAGGAVHTRGLDFACDIYIWAPLAGT |
| | | CGVLLLSLVITLYCNHRNRRRVCKCPRVDRSKRSRLLHSDYMNMTPRRPGPTR |
| | | KHYQPYAPPRDFAAYRSRVKFSRSADAPAYQQGQNQLYNELNLGRREEYDVLD |
| | | KRRGRDPEMGGKPRRKNPQEGLYNELQKDKMAEAYSEIGMKGERRRGKGHDGL |
| | | YQGLSTATKDTYDALHMQALPPR |
| | | Structural formula: Nterm-(Linker 1)(CD34 tag)- |
| | | (Linker 2)(VH Domain)(Linker 3)(VL Domain)(Linker |
| | | 4)(CD8 stalk region)(CD8 transmembrane region)- |
| | | (Linker 5)(CD28 cytoplasmic region)(CD3-zeta |
| | | cytoplasmic region)-Cterm |
| | | X is I or V |
| Binding Molecule E | 198 | GSELPTQGTFSNVSTNVSSPEVKLVESGGGLVKPGGSLKVSCTASGFTFSRYA |
| CAR polypeptide (without | | MSWVRQTPERRLEWVASITFGGSAYYLDSVKGRFTISRDNAQNILYLQMNSLV |
| signal region) | | SEDTAIYNCARHQPWFDYWGQGTLVTVSAGGGGSGGGGSGGGGSDVLLTQTPL |
| | | SLPVSLGDQASISCRSSQNIVHSDGDTYLDWFLQKPGQSPNLLIYKVSNRFSG |
| | | VPDRFSGSGSGTDFTLKISRVEADDLGVYYCFQASHVPYTFGGGTKLEVKRTT |
| | | TPAPRPPTPAPTIASQPLSLRPEACRPAAGGAVHTRGLDFACDIYIWAPLAGT |
| | | CGVLLLSLVITLYCNHRNRRRVCKCPRVDRSKRSRLLHSDYMNMTPRRPGPTR |
| | | KHYQPYAPPRDFAAYRSRVKFSRSADAPAYQQGQNQLYNELNLGRREEYDVLD |
| | | KRRGRDPEMGGKPRRKNPQEGLYNELQKDKMAEAYSEIGMKGERRRGKGHDGL |
| | | YQGLSTATKDTYDALHMQALPPR |
| | | Structural formula: Nterm-(Linker 1)(CD34 tag)- |
| | | (Linker 2)(VH Domain)(Linker 3)(VL Domain)(Linker |
| | | 4)(CD8 stalk region)(CD8 transmembrane region)- |
| | | (Linker 5)(CD28 cytoplasmic region)(CD3-zeta |
| | | cytoplasmic region)-Cterm |
| chimeric PD1-A | 199 | FLDSPDRPWNPPTESPALLVVTEGDNATFTCSESNTSESFVLNWYRMSPSNQT |
| polypeptide (without signal | | DKLAAFPEDRSQPGQDCRFRVTQLPNGRDFHMSVVRARRNDSGTYLCGAISLA |
| region) | | PKAQIKESLRAELRVTERRAEVPTAHPSPSPRPAGQFQTLVCPSPLFPGPSKP |
| | | FWVLVVVGGVLACYSLLVTVAFIIFWVLCARPRRSPAQEDGKVYINMPGRGRV |
| | | KFSRSADAPAYQQGQNQLYNELNLGRREEYDVLDKRRGRDPEMGGKPQRRKNP |
| | | QEGLYNELQKDKMAEAYSEIGMKGERRRGKGHDGLYQGLSTATKDTYDALHMQ |
| | | ALPPR |
| | | Structural formula: Nterm-(PD1 region |
| | | (extracellular))(truncated CD28 region |
| | | (extracellular))(CD28 transmembrane region)(DAP10 |
| | | region (cytoplasmic))(CD3-zeta region |
| | | (cytoplasmic))-Cterm |
| chimeric PD1-B | 200 | GSELPTQGTFSNVSTNVSSPGSPGWFLDSPDRPWNPPTFSPALLVVTEGDNAT |
| polypeptide (without signal | | FTCSFSNTSESFVLNWYRMSPSNQTDKLAAFPEDRSQPGQDCRFRVTQLPNGR |
| region) | | DFHMSVVRARRNDSGTYLCGAISLAPKAQIKESLRAELRVTERRAEVPTAHPS |
| | | PSPRPAGQFQTLVCPSPLFPGPSKPFWVLVVVGGVLACYSLLVTVAFIIFWVL |
| | | CARPRRSPAQEDGKVYINMPGRGRVKFSRSADAPAYQQGQNQLYNELNLGRRE |
| | | EYDVLDKRRGRDPEMGGKPQRRKNPQEGLYNELQKDKMAEAYSEIGMKGERRR |
| | | GKGHDGLYQGLSTATKDTYDALHMQALPPR |
| | | Structural formula: Nterm-(CD34 tag)(linker)(PD1 |
| | | region (extracellular))(truncated CD28 region |
| | | (extracellular))(CD28 transmembrane region)(DAP10 |
| | | region (cytoplasmic))(CD3-zeta region |
| | | (cytoplasmic))-Cterm |
| uc002cjy | 201 | EDIRKWNVTSLETLKALLEVNKGHEMSPQVATLIDRFVKGRGQLDKDTLD |
| uc002cjv | 202 | EDIRKWNVTSLETLKALLEVNKGHEMSPQVATLIDRFVKGRGQLDKDTLD |
| uc010brd | 203 | EDIRKWNVTSLETLKALLEVNKGHEMSPQVATLIDRFVKGRGQLDKDTLD |
| uc002cjt | 204 | EDIRKWNVTSLETLKALLEVNKGHEMSPQVATLIDRFVKGRGQLDKDTLD |
| uc002cju | 205 | EDIRKWNVTSLETLKALLEVNKGHEMSPQVATLIDRFVKGRGQLDKDTLD |
| uc002cjx | 206 | EDIRKWNVTSLETLKALLEVNKGHEMSPQVATLIDRFVKGRGQLDKDTLD |
| uc002cjw | 207 | EDIRKWNVTSLETLKALLEVNKGHEMSPQAPRRPLPQVATLIDRFVKGRGQLD |
| | | KDTLD |

pKB113 plasmid (SEQ ID NO: 217):
TGCCCCGGCTCAGGGCCAAGAACAGATGGAACAGCTGAATATGGGCCAAACAGGATATCTGTGGTAAGCAGTTCCTG
CCCCGGCTCAGGGCCAAGAACAGATGGTCCCCAGATGCGGTCCAGCCCTCAGCAGTTTCTAGAGAACCATCAGATGT
TTCCAGGGTGCCCCAAGGACCTGAAATGACCCTGTGCCTTATTTGAACTAACCAATCAGTTCGCTTCTCGCTTCTGT
TCGCGCGCTTATGCTCCCCGAGCTCAATAAAAGAGCCCACAACCCCTCACTCGGGGCGCCAGTCCTCCGATTGACTG
AGTCGCCCGGGTACCCGTGTATCCAATAAACCCTCTTGCAGTTGCATCCGACTTGTGGTCTCGCTGTTCCTTGGGAG
GGTCTCCTCTGAGTGATTGACTACCCGTCAGCGGGGGTCTTTCATTTGGGGGCTCGTCCGGGATCGGGAGACCCCTG
CCCAGGGACCACCGACCCACCACCGGGAGGTAAGCTGGCCAGCAACTTATCTGTGTCTGTCCGATTGTCTAGTGTCT
ATGACTGATTTTATGCGCCTGCGTCGGTACTAGTTAGCTAACTAGCTCTGTATCTGGCGGACCCGTGGTGGAACTGA
CGAGTTCGGAACACCCGGCCGCAACCCTGGGAGACGTCCCAGGGACTTCGGGGGCCGTTTTTGTGGCCCGACCTGAG
TCCTAAAATCCCGATCGTTTAGGACTCTTTGGTGCACCCCCCTTAGAGGAGGGATATGTGGTTCTGGTAGGAGACGA
GAACCTAAAACAGTTCCCGCCTCCGTCTGAATTTTTGCTTTCGGTTTGGGACCGAAGCCGCGCCGCGCGTCTTGTCT
GCTGCAGCATCGTTCTGTGTTGTCTCTGTCTGACTGTGTTTCTGTATTTGTCTGAAAATATGGGCCCGGGCTAGCCT
GTTACCACTCCCTTAAGTTTGACCTTAGGTCACTGGAAAGATGTCGAGCGGATCGCTCACAACCAGTCGGTAGATGT
CAAGAAGAGACGTTGGGTTACCTTCTGCTCTGCAGAATGGCCAACCTTTAACGTCGGATGGCCGCGAGACGGCACCT
TTAACCGAGACCTCATCACCCAGGTTAAGATCAAGGTCTTTTCACCTGGCCCGCATGGACACCCAGACCAGGTGGGG
TACATCGTGACCTGGGAAGCCTTGGCTTTTGACCCCCCTCCCTGGGTCAAGCCCTTTGTACACCCTAAGCCTCCGCC
TCCTCTTCCTCCATCCGCCCCGTCTCTCCCCCTTGAACCTCCTCGTTCGACCCCGCCTCGATCCTCCCTTTATCCAG
CCCTCACTCCTTCTCTAGGCGCCCCCATATGGCCATATGAGATCTTATATGGGGCACCCCCGCCCCTTGTAAACTTC
CCTGACCCTGACATGACAAGAGTTACTAACAGCCCCTCTCTCCAAGCTCACTTACAGGCTCTCTACTTAGTCCAGCA
CGAAGTCTGGAGACCTCTGGCGGCAGCCTACCAAGAACAACTGGACCGACCGGTGGTACCTCACCCTTACCGAGTCG
GCGACACAGTGTGGGTCCGCCGACACCAGACTAAGAACCTAGAACCTCGCTGGAAAGGACCTTACACAGTCCTGCTG
ACCACCCCCACCGCCCTCAAAGTAGACGGCATCGCAGCTTGGATACGACGCCGCCCACGTGAAGGCTGCCGACCCCG
GGGGTGGACCATCCTCTAGACTGCCAGCGGCCGCTCGAGCCACCATGGCTCTTCCGGTAACCGCTCTGCTGCTTCCT
TTGGCTCTGCTTCTTCACGCGGCTAGGCCAGGGAGCGAATTGCCTACGCAGGGGACTTTTTCAAATGTAAGCACGAA
TGTGAGCTCTCCGGAAGTGAAATTGGTAGAGTCTGGTGGGGGACTGGTTAAACCTGGAGGCAGTCTGAAGCTCTCCT
GCGAAGCCAGCGGCTTTACGTTCAGTCGGTACGCTATGTCCTGGGTGCGACACACACCAGAAAAGCGACTCGCTTGG
GTAGCGAGCATAACGTTTGGGGGCGATCCTTATTACCCGGATGGTTTGAAAGGTCGCTTCACTATCAGTCGAGATAA
TACGCGAAACATTCTTTATCTTCAAATGAATAGCCTGCGCTCTGAAGACACCGCGATGTATTATTGTGTTAGGCATG
AGTCTTGGTTTGCATACTGGGGTCAGGGTACTCTTGTCACTGTATCTGCCGGAGGTGGGGGTAGCGGCGGCGGAGGT
AGTGGGGGAGGCGGCAGCGACGTGTTGATGACTCAAACTCCGCTTAGCTTGCCAGTGTCTCTTGGAGAACAGGTCTC
CATATCATGTCGATCCAGTCAAACTATCGTTCATACCGATGGGAATATCTATCTCGAATGGTATCTGCAAAATCCAG
GGCAGAGTCCCCGGCTTCTGATATATAAGATTAGCAATAGATTTAGTGGTGTTCCCGACAGGTTTTCAGGTTCTGGG
TCCGGAACGGATTTTACCCTGAAGATAAGTCGAGTTGAAGCGGAAGACCTGGGCATATACTATTGCTTCCAGGCGTC
CCACGTCCCCTATACATTTGGGGGTGGGACTAAACTGGAAATTAAACGTACGACGACACCAGCCCCAAGACCCCCAA
CTCCCGCTCCTACCATAGCTTCCCAACCCCTGTCACTGAGGCCAGAGGCATGCAGGCCCGCTGCGGGTGGCGCGGTA
CATACGCGGGGACTGGACTTTGCATGTGACATTTACATATGGGCGCCACTGGCGGGAACATGTGGAGTTTTGTTGCT
TAGCCTGGTCATAACACTGTATTGCAATCATCGCAACAGACGCCGCGTCTGCAAGTGTCCCAGGGTCGACAGATCTA
AGAGAAGTAGACTTCTTCACAGTGATTATATGAATATGACGCCTCGAAGACCCGGCCCGACACGCAAACACTATCAG
CCGTATGCCCCCCCTCGGGATTTTGCTGCCTACCGCAGCCGCGTTAAGTTCTCTAGGTCCGCTGACGCCCCTGCCTA
CCAGCAGGGTCAAAACCAACTGTACAATGAATTGAATCTTGGGAGACGGGAGGAGTATGACGTACTCGACAAGCGGA
GGGGGAGAGATCCTGAGATGGGTGGAAAGCCTCGACGAAAAAACCCACAAGAAGGGTTGTATAATGAACTGCAGAAA
GACAAGATGGCTGAGGCATATAGTGAAATCGGGATGAAGGGAGAACGCCGGCGCGGGAAAGGGCACGATGGTCTGTA
TCAAGGGCTTAGTACGGCTACGAAGGACACATATGACGCTTTGCACATGCAGGCCCTTCCTCCTAGGTGAACGCGTC
ATCATCGATCCGGATTAGGATCGATCCGTCCAATTTGTTAAAGACAGGATATCAGTGGTCCAGGCTCTAGTTTTGAC
TCAACAATATCACCAGCTGAAGCCTATAGAGTACGAGCCATAGATAAAATAAAAGATTTTATTTAGTCTCCAGAAAA
AGGGGGGAATGAAAGACCCCACCTGTAGGTTTGGCAAGCTAGCTTAAGTAACGCCATTTTGCAAGGCATGGAAAAAT
ACATAACTGAGAATAGAGAAGTTCAGATCAAGGTCAGGAACAGATGGAACAGCTGAATATGGGCCAAACAGGATATC
TGTGGTAAGCAGTTCCTGCCCCGGCTCAGGGCCAAGAACAGATGGAACAGCTGAATATGGGCCAAACAGGATATCTG
TGGTAAGCAGTTCCTGCCCCGGCTCAGGGCCAAGAACAGATGGTCCCCAGATGCGGTCCAGCCCTCAGCAGTTTCTA
GAGAACCATCAGATGTTTCCAGGGTGCCCCAAGGACCTGAAATGACCCTGTGCCTTATTTGAACTAACCAATCAGTT
CGCTTCTCGCTTCTGTTCGCGCGCTTCTGCTCCCCGAGCTCAATAAAAGAGCCCACAACCCCTCACTCGGGGCGCCA
GTCCTCCGATTGACTGAGTCGCCCGGGTACCCGTGTATCCAATAAACCCTCTTGCAGTTGCATCCGACTTGTGGTCT
CGCTGTTCCTTGGGAGGGTCTCCTCTGAGTGATTGACTACCCGTCAGCGGGGGTCTTTCACACATGCAGCATGTATC
AAAATTAATTTGGTTTTTTTTCTTAAGTATTTACATTAAATGGCCATAGTACTTAAAGTTACATTGGCTTCCTTGAA
ATAAACATGGAGTATTCAGAATGTGTCATAAATATTTCTAATTTTAAGATAGTATCTCCATTGGCTTTCTACTTTTT
CTTTTATTTTTTTTTGTCCTCTGTCTTCCATTTGTTGTTGTTGTTGTTTGTTTGTTTGTTTGTTGGTTGGTTGGTTA
ATTTTTTTTTAAAGATCCTACACTATAGTTCAAGCTAGACTATTAGCTACTCTGTAACCCAGGGTGACCTTGAAGTC
ATGGGTAGCCTGCTGTTTTAGCCTTCCCACATCTAAGATTACAGGTATGAGCTATCATTTTTGGTATATTGATTGAT
TGATTGATTGATGTGTGTGTGTGTGATTGTGTTTGTGTGTGTGACTGTGAAAATGTGTGTATGGGTGTGTGTGAATG
TGTGTATGTATGTGTGTGTGTGAGTGTGTGTGTGTGTGTGTGCATGTGTGTGTGTGTGACTGTGTCTATGTGTATGA
CTGTGTGTGTGTGTGTGTGTGTGTGTGTGTGTGTGTGTGTGTGTGTGTTGTGAAAAAATATTCTATGGTAGTGAGAG
CCAACGCTCCGGCTCAGGTGTCAGGTTGGTTTTTGAGACAGAGTCTTTCACTTAGCTTGGAATTCACTGGCCGTCGT
TTTACAACGTCGTGACTGGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAATGGCGAATGGCGCCTGATG
CGGTATTTTCTCCTTACGCATCTGTGCGGTATTTCACACCGCATATGGTGCACTCTCAGTACAATCTGCTCTGATGC
CGCATAGTTAAGCCAGCCCCGACACCCGCCAACACCCGCTGACGCGCCCTGACGGGCTTGTCTGCTCCCGGCATCCG
CTTACAGACAAGCTGTGACCGTCTCCGGGAGCTGCATGTGTCAGAGGTTTTCACCGTCATCACCGAAACGCGCGATG
ACGAAAGGGCCTCGTGATACGCCTATTTTTATAGGTTAATGTCATGATAATAATGGTTTCTTAGACGTCAGGTGGCA
CTTTTCGGGGAAATGTGCGCGGAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAGTAAAAGATGCTGAAGATC
AGTTGGGTGCACGAGTGGGTTACATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCCGTATTGACGCCGGGCAAGAGCA
ACTCGGTCGCCGCATACACTATTCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAACTCGCCTTGATCGTTGGGAACC
GGAGCTGAATGAAGCCATACCAAACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGTGGGTCTCGCGG
TATCATTGCAGCACTGGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTGAAGATCCTTTTTGATAATCT
CATGACCAAAATCCCTTAACGTGAGTTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACTGAGATACCTACAGCG
TGAGCATTGAGAAAGCGCCACGCTTCCCGAAGGGAGAAAGGCGGACAGGTATCCGGTAAGCGGCAGGGTCGGAACAG
GAGAGCGCACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTTTACGGTT
CCTGGCCTTTTGCTGGCCTTTTGCTCACATGTTCTTTCCTGCGTTATCCCCTGATTCTGTGGATAACCGTATTACCG
CCTTTGAGTGAGCTGATACCGCTCGCCGCAGCCGAACGACCGAGCGCAGCGAGTCAGTGAGCGAGGAAGCGGAAGAG
CGCCCAATACGCAAACCGCCTCTCCCCGCGCGTTGGCCGATTCATTAATGCAGCTGGCACGACAGGTTTCCCGACTG
GAAAGCGGGCAGTGAGCGCAACGCAATTAATGTGAGTTAGCTCACTCATTAGGCACCCCAGGCTTTACACTTTATGC
TTCCGGCTCGTATGTTGTGTGGAATTGTGAGCGGATAACAATTTCACACAGGAAACAGCTATGACCATGATTACGCC
AAGCTTTGCTCTTAGGAGTTTCCTAATACATCCCAAACTCAAATATATAAAGCATTTGACTTGTTCTATGCCCTAGG
GGGCGGGGGGAAGCTAAGCCAGCTTTTTTTAACATTTAAAATGTTAATTCCATTTTAAATGCACAGATGTTTTTATT
TCATAAGGGTTTCAATGTGCATGAATGCTGCAATATTCCTGTTACCAAAGCTAGTATAAATAAAAATAGATAAACGT
GGAAATTACTTAGAGTTTCTGTCATTAACGTTTCCTTCCTCAGTTGACAACATAAATGCGCTGCTGAGCAAGCCAGT
TTGCATCTGTCAGGATCAATTTCCCATTATGCCAGTCATATTAATTACTAGTCAATTAGTTGATTTTTATTTTTGAC
ATATACATGTGATGAAAGACCCCACCTGTAGGTTTGGCAAGCTAGCTTAAGTAACGCCATTTTGCAAGGCATGGAAA
AATACATAACTGAGAATAGAAAAGTTCAGATCAAGGTCAGGAACAGATGGAACAGCTGAATATGGGCCAAACAGGAT
ATCTGTGGTAAGCAGTTCC
pKB115 plasmid (SEQ ID NO: 218):
TGCCCCGGCTCAGGGCCAAGAACAGATGGAACAGCTGAATATGGGCCAAACAGGATATCTGTGGTAAGCAGTTCCTG
CCCCGGCTCAGGGCCAAGAACAGATGGTCCCCAGATGCGGTCCAGCCCTCAGCAGTTTCTAGAGAACCATCAGATGT
TTCCAGGGTGCCCCAAGGACCTGAAATGACCCTGTGCCTTATTTGAACTAACCAATCAGTTCGCTTCTCGCTTCTGT
TCGCGCGCTTATGCTCCCCGAGCTCAATAAAAGAGCCCACAACCCCTCACTCGGGGCGCCAGTCCTCCGATTGACTG
AGTCGCCCGGGTACCCGTGTATCCAATAAACCCTCTTGCAGTTGCATCCGACTTGTGGTCTCGCTGTTCCTTGGGAG
GGTCTCCTCTGAGTGATTGACTACCCGTCAGCGGGGGTCTTTCATTTGGGGGCTCGTCCGGGATCGGGAGACCCCTG
CCCAGGGACCACCGACCCACCACCGGGAGGTAAGCTGGCCAGCAACTTATCTGTGTCTGTCCGATTGTCTAGTGTCT
ATGACTGATTTTATGCGCCTGCGTCGGTACTAGTTAGCTAACTAGCTCTGTATCTGGCGGACCCGTGGTGGAACTGA
CGAGTTCGGAACACCCGGCCGCAACCCTGGGAGACGTCCCAGGGACTTCGGGGGCCGTTTTTGTGGCCCGACCTGAG
TCCTAAAATCCCGATCGTTTAGGACTCTTTGGTGCACCCCCCTTAGAGGAGGGATATGTGGTTCTGGTAGGAGACGA
GAACCTAAAACAGTTCCCGCCTCCGTCTGAATTTTTGCTTTCGGTTTGGGACCGAAGCCGCGCCGCGCGTCTTGTCT
GCTGCAGCATCGTTCTGTGTTGTCTCTGTCTGACTGTGTTTCTGTATTTGTCTGAAAATATGGGCCCGGGCTAGCCT
GTTACCACTCCCTTAAGTTTGACCTTAGGTCACTGGAAAGATGTCGAGCGGATCGCTCACAACCAGTCGGTAGATGT
CAAGAAGAGACGTTGGGTTACCTTCTGCTCTGCAGAATGGCCAACCTTTAACGTCGGATGGCCGCGAGACGGCACCT
TTAACCGAGACCTCATCACCCAGGTTAAGATCAAGGTCTTTTCACCTGGCCCGCATGGACACCCAGACCAGGTGGGG
TACATCGTGACCTGGGAAGCCTTGGCTTTTGACCCCCCTCCCTGGGTCAAGCCCTTTGTACACCCTAAGCCTCCGCC
TCCTCTTCCTCCATCCGCCCCGTCTCTCCCCCTTGAACCTCCTCGTTCGACCCCGCCTCGATCCTCCCTTTATCCAG
CCCTCACTCCTTCTCTAGGCGCCCCCATATGGCCATATGAGATCTTATATGGGGCACCCCCGCCCCTTGTAAACTTC
CCTGACCCTGACATGACAAGAGTTACTAACAGCCCCTCTCTCCAAGCTCACTTACAGGCTCTCTACTTAGTCCAGCA
CGAAGTCTGGAGACCTCTGGCGGCAGCCTACCAAGAACAACTGGACCGACCGGTGGTACCTCACCCTTACCGAGTCG
GCGACACAGTGTGGGTCCGCCGACACCAGACTAAGAACCTAGAACCTCGCTGGAAAGGACCTTACACAGTCCTGCTG
ACCACCCCCACCGCCCTCAAAGTAGACGGCATCGCAGCTTGGATACGACGCCGCCCACGTGAAGGCTGCCGACCCCG
GGGGTGGACCATCCTCTAGACTGCCAGCGGCCGCTCGAGCCACCATGGCTCTTCCGGTAACCGCTCTGCTGCTTCCT
TTGGCTCTGCTTCTTCACGCGGCTAGGCCAGGGAGCGAATTGCCTACGCAGGGGACTTTTTCAAATGTAAGCACGAA
TGTGAGCTCTCCGGAAGTCAAATTGGTGGAGAGTGGCGGTGGGCTCGTAAAACCAGGAGGCAGCTTGAAAGTAAGTT
GCACAGCGTCTGGGTTCACTTTCAGCCGATACGCTATGAGTTGGGTGCGGCAAACGCCCGAAAGACGATTGGAGTGG
GTAGCTAGTATTACTTTTGGGGGTTCCGCGTATTACTTGGATAGCGTTAAAGGGAGATTCACCATATCACGAGATAA
TGCCCAAAACATACTCTACCTCCAGATGAACTCATTGGTGTCTGAAGATACTGCAATTTACAACTGCGCCAGGCACC
AGCCATGGTTTGACTATTGGGGTCAAGGTACGTTGGTCACAGTCTCAGCCGGCGGTGGCGGATCTGGAGGGGGAGGC
AGTGGAGGAGGTGGTTCAGATGTTCTCCTGACGCAAACTCCTTTGTCATTGCCGGTAAGTCTGGGCGACCAAGCGAG
CATTTCCTGTCGCAGCAGCCAGAACATAGTTCATAGTGATGGCGACACTTACTTGGATTGGTTCCTCCAGAAACCGG
GGCAATCTCCGAACCTCTTGATATATAAGGTCTCAAATCGCTTTAGCGGGGTTCCCGACCGCTTTTCAGGTTCTGGC
TCAGGTACCGATTTCACGCTGAAAATTTCTAGAGTAGAGGCTGACGATCTGGGTGTTTACTATTGTTTTCAAGCTTC
ACACGTTCCTTATACGTTCGGAGGGGGGACTAAACTGGAGGTTAAACGTACGACGACACCAGCCCCAAGACCCCCAA
CTCCCGCTCCTACCATAGCTTCCCAACCCCTGTCACTGAGGCCAGAGGCATGCAGGCCCGCTGCGGGTGGCGCGGTA
CATACGCGGGGACTGGACTTTGCATGTGACATTTACATATGGGCGCCACTGGCGGGAACATGTGGAGTTTTGTTGCT
TAGCCTGGTCATAACACTGTATTGCAATCATCGCAACAGACGCCGCGTCTGCAAGTGTCCCAGGGTCGACAGATCTA
AGAGAAGTAGACTTCTTCACAGTGATTATATGAATATGACGCCTCGAAGACCCGGCCCGACACGCAAACACTATCAG
CCGTATGCCCCCCCTCGGGATTTTGCTGCCTACCGCAGCCGCGTTAAGTTCTCTAGGTCCGCTGACGCCCCTGCCTA
CCAGCAGGGTCAAAACCAACTGTACAATGAATTGAATCTTGGGAGACGGGAGGAGTATGACGTACTCGACAAGCGGA
GGGGGAGAGATCCTGAGATGGGTGGAAAGCCTCGACGAAAAAACCCACAAGAAGGGTTGTATAATGAACTGCAGAAA
GACAAGATGGCTGAGGCATATAGTGAAATCGGGATGAAGGGAGAACGCCGGCGCGGGAAAGGGCACGATGGTCTGTA
TCAAGGGCTTAGTACGGCTACGAAGGACACATATGACGCTTTGCACATGCAGGCCCTTCCTCCTAGGTGAACGCGTC
ATCATCGATCCGGATTAGGATCGATCCGTCCAATTTGTTAAAGACAGGATATCAGTGGTCCAGGCTCTAGTTTTGAC
TCAACAATATCACCAGCTGAAGCCTATAGAGTACGAGCCATAGATAAAATAAAAGATTTTATTTAGTCTCCAGAAAA
AGGGGGGAATGAAAGACCCCACCTGTAGGTTTGGCAAGCTAGCTTAAGTAACGCCATTTTGCAAGGCATGGAAAAAT
ACATAACTGAGAATAGAGAAGTTCAGATCAAGGTCAGGAACAGATGGAACAGCTGAATATGGGCCAAACAGGATATC
TGTGGTAAGCAGTTCCTGCCCCGGCTCAGGGCCAAGAACAGATGGAACAGCTGAATATGGGCCAAACAGGATATCTG
TGGTAAGCAGTTCCTGCCCCGGCTCAGGGCCAAGAACAGATGGTCCCCAGATGCGGTCCAGCCCTCAGCAGTTTCTA
GAGAACCATCAGATGTTTCCAGGGTGCCCCAAGGACCTGAAATGACCCTGTGCCTTATTTGAACTAACCAATCAGTT
CGCTTCTCGCTTCTGTTCGCGCGCTTCTGCTCCCCGAGCTCAATAAAAGAGCCCACAACCCCTCACTCGGGGCGCCA
GTCCTCCGATTGACTGAGTCGCCCGGGTACCCGTGTATCCAATAAACCCTCTTGCAGTTGCATCCGACTTGTGGTCT
CGCTGTTCCTTGGGAGGGTCTCCTCTGAGTGATTGACTACCCGTCAGCGGGGGTCTTTCACACATGCAGCATGTATC
AAAATTAATTTGGTTTTTTTTCTTAAGTATTTACATTAAATGGCCATAGTACTTAAAGTTACATTGGCTTCCTTGAA
ATAAACATGGAGTATTCAGAATGTGTCATAAATATTTCTAATTTTAAGATAGTATCTCCATTGGCTTTCTACTTTTT
CTTTTATTTTTTTTTGTCCTCTGTCTTCCATTTGTTGTTGTTGTTGTTTGTTTGTTTGTTTGTTGGTTGGTTGGTTA
ATTTTTTTTTAAAGATCCTACACTATAGTTCAAGCTAGACTATTAGCTACTCTGTAACCCAGGGTGACCTTGAAGTC
ATGGGTAGCCTGCTGTTTTAGCCTTCCCACATCTAAGATTACAGGTATGAGCTATCATTTTTGGTATATTGATTGAT
TGATTGATTGATGTGTGTGTGTGTGATTGTGTTTGTGTGTGTGACTGTGAAAATGTGTGTATGGGTGTGTGTGAATG
TGTGTATGTATGTGTGTGTGTGAGTGTGTGTGTGTGTGTGTGCATGTGTGTGTGTGTGACTGTGTCTATGTGTATGA
CTGTGTGTGTGTGTGTGTGTGTGTGTGTGTGTGTGTGTGTGTGTGTGTTGTGAAAAAATATTCTATGGTAGTGAGAG
CCAACGCTCCGGCTCAGGTGTCAGGTTGGTTTTTGAGACAGAGTCTTTCACTTAGCTTGGAATTCACTGGCCGTCGT
TTTACAACGTCGTGACTGGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAATGGCGAATGGCGCCTGATG
CGGTATTTTCTCCTTACGCATCTGTGCGGTATTTCACACCGCATATGGTGCACTCTCAGTACAATCTGCTCTGATGC
CGCATAGTTAAGCCAGCCCCGACACCCGCCAACACCCGCTGACGCGCCCTGACGGGCTTGTCTGCTCCCGGCATCCG
CTTACAGACAAGCTGTGACCGTCTCCGGGAGCTGCATGTGTCAGAGGTTTTCACCGTCATCACCGAAACGCGCGATG
ACGAAAGGGCCTCGTGATACGCCTATTTTTATAGGTTAATGTCATGATAATAATGGTTTCTTAGACGTCAGGTGGCA
CTTTTCGGGGAAATGTGCGCGGAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAGTAAAAGATGCTGAAGATC
AGTTGGGTGCACGAGTGGGTTACATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCCGTATTGACGCCGGGCAAGAGCA
ACTCGGTCGCCGCATACACTATTCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAACTCGCCTTGATCGTTGGGAACC
GGAGCTGAATGAAGCCATACCAAACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGTGGGTCTCGCGG
TATCATTGCAGCACTGGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTGAAGATCCTTTTTGATAATCT
CATGACCAAAATCCCTTAACGTGAGTTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACTGAGATACCTACAGCG
TGAGCATTGAGAAAGCGCCACGCTTCCCGAAGGGAGAAAGGCGGACAGGTATCCGGTAAGCGGCAGGGTCGGAACAG
GAGAGCGCACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTTTACGGTT
CCTGGCCTTTTGCTGGCCTTTTGCTCACATGTTCTTTCCTGCGTTATCCCCTGATTCTGTGGATAACCGTATTACCG
CCTTTGAGTGAGCTGATACCGCTCGCCGCAGCCGAACGACCGAGCGCAGCGAGTCAGTGAGCGAGGAAGCGGAAGAG
CGCCCAATACGCAAACCGCCTCTCCCCGCGCGTTGGCCGATTCATTAATGCAGCTGGCACGACAGGTTTCCCGACTG
GAAAGCGGGCAGTGAGCGCAACGCAATTAATGTGAGTTAGCTCACTCATTAGGCACCCCAGGCTTTACACTTTATGC
TTCCGGCTCGTATGTTGTGTGGAATTGTGAGCGGATAACAATTTCACACAGGAAACAGCTATGACCATGATTACGCC
AAGCTTTGCTCTTAGGAGTTTCCTAATACATCCCAAACTCAAATATATAAAGCATTTGACTTGTTCTATGCCCTAGG
GGGCGGGGGGAAGCTAAGCCAGCTTTTTTTAACATTTAAAATGTTAATTCCATTTTAAATGCACAGATGTTTTTATT
TCATAAGGGTTTCAATGTGCATGAATGCTGCAATATTCCTGTTACCAAAGCTAGTATAAATAAAAATAGATAAACGT
GGAAATTACTTAGAGTTTCTGTCATTAACGTTTCCTTCCTCAGTTGACAACATAAATGCGCTGCTGAGCAAGCCAGT
TTGCATCTGTCAGGATCAATTTCCCATTATGCCAGTCATATTAATTACTAGTCAATTAGTTGATTTTTATTTTTGAC
ATATACATGTGATGAAAGACCCCACCTGTAGGTTTGGCAAGCTAGCTTAAGTAACGCCATTTTGCAAGGCATGGAAA
AATACATAACTGAGAATAGAAAAGTTCAGATCAAGGTCAGGAACAGATGGAACAGCTGAATATGGGCCAAACAGGAT
ATCTGTGGTAAGCAGTTCC
chPD1 pSFG plasmid (encoding ″Chimeric PD1 Molecule A″) (SEQ ID NO: 219):
TGCCCCGGCTCAGGGCCAAGAACAGATGGAACAGCTGAATATGGGCCAAACAGGATATCTGTGGTAAGCAGTTCCTG
CCCCGGCTCAGGGCCAAGAACAGATGGTCCCCAGATGCGGTCCAGCCCTCAGCAGTTTCTAGAGAACCATCAGATGT
TTCCAGGGTGCCCCAAGGACCTGAAATGACCCTGTGCCTTATTTGAACTAACCAATCAGTTCGCTTCTCGCTTCTGT
TCGCGCGCTTATGCTCCCCGAGCTCAATAAAAGAGCCCACAACCCCTCACTCGGGGCGCCAGTCCTCCGATTGACTG
AGTCGCCCGGGTACCCGTGTATCCAATAAACCCTCTTGCAGTTGCATCCGACTTGTGGTCTCGCTGTTCCTTGGGAG
GGTCTCCTCTGAGTGATTGACTACCCGTCAGCGGGGGTCTTTCATTTGGGGGCTCGTCCGGGATCGGGAGACCCCTG
CCCAGGGACCACCGACCCACCACCGGGAGGTAAGCTGGCCAGCAACTTATCTGTGTCTGTCCGATTGTCTAGTGTCT
ATGACTGATTTTATGCGCCTGCGTCGGTACTAGTTAGCTAACTAGCTCTGTATCTGGCGGACCCGTGGTGGAACTGA
CGAGTTCGGAACACCCGGCCGCAACCCTGGGAGACGTCCCAGGGACTTCGGGGGCCGTTTTTGTGGCCCGACCTGAG
TCCTAAAATCCCGATCGTTTAGGACTCTTTGGTGCACCCCCCTTAGAGGAGGGATATGTGGTTCTGGTAGGAGACGA
GAACCTAAAACAGTTCCCGCCTCCGTCTGAATTTTTGCTTTCGGTTTGGGACCGAAGCCGCGCCGCGCGTCTTGTCT
GCTGCAGCATCGTTCTGTGTTGTCTCTGTCTGACTGTGTTTCTGTATTTGTCTGAAAATATGGGCCCGGGCTAGCCT
GTTACCACTCCCTTAAGTTTGACCTTAGGTCACTGGAAAGATGTCGAGCGGATCGCTCACAACCAGTCGGTAGATGT
CAAGAAGAGACGTTGGGTTACCTTCTGCTCTGCAGAATGGCCAACCTTTAACGTCGGATGGCCGCGAGACGGCACCT
TTAACCGAGACCTCATCACCCAGGTTAAGATCAAGGTCTTTTCACCTGGCCCGCATGGACACCCAGACCAGGTGGGG
TACATCGTGACCTGGGAAGCCTTGGCTTTTGACCCCCCTCCCTGGGTCAAGCCCTTTGTACACCCTAAGCCTCCGCC
TCCTCTTCCTCCATCCGCCCCGTCTCTCCCCCTTGAACCTCCTCGTTCGACCCCGCCTCGATCCTCCCTTTATCCAG
CCCTCACTCCTTCTCTAGGCGCCCCCATATGGCCATATGAGATCTTATATGGGGCACCCCCGCCCCTTGTAAACTTC
CCTGACCCTGACATGACAAGAGTTACTAACAGCCCCTCTCTCCAAGCTCACTTACAGGCTCTCTACTTAGTCCAGCA
CGAAGTCTGGAGACCTCTGGCGGCAGCCTACCAAGAACAACTGGACCGACCGGTGGTACCTCACCCTTACCGAGTCG
GCGACACAGTGTGGGTCCGCCGACACCAGACTAAGAACCTAGAACCTCGCTGGAAAGGACCTTACACAGTCCTGCTG
ACCACCCCCACCGCCCTCAAAGTAGACGGCATCGCAGCTTGGATACGACGCCGCCCACGTGAAGGCTGCCGACCCCG
GGGGTGGACCATCCTCTAGACTGCCAGCGGCCGCGCCACCATGCAGATCCCCCAGGCCCCCTGGCCCGTGGTGTGGG
CCGTGCTGCAGCTGGGCTGGAGGCCCGGCTGGTTCCTGGACAGCCCCGACAGGCCCTGGAACCCCCCCACCTTCAGC
CCCGCCCTGCTGGTGGTGACCGAGGGCGACAACGCCACCTTCACCTGCAGCTTCAGCAACACCAGCGAGAGCTTCGT
GCTGAACTGGTACAGGATGAGCCCCAGCAACCAGACCGACAAGCTGGCCGCCTTCCCCGAGGACAGGAGCCAGCCCG
GCCAGGACTGCAGGTTCAGGGTGACCCAGCTGCCCAACGGCAGGGACTTCCACATGAGCGTGGTGAGGGCCAGGAGG
AACGACAGCGGCACCTACCTGTGCGGCGCCATCAGCCTGGCCCCCAAGGCCCAGATCAAGGAGAGCCTGAGGGCCGA
GCTGAGGGTGACCGAGAGGAGGGCCGAGGTGCCCACCGCCCACCCCAGCCCCAGCCCCAGGCCCGCCGGCCAGTTCC
AGACCCTGGTGTGCCCCAGCCCCCTGTTCCCCGGCCCCAGCAAGCCCTTCTGGGTGCTGGTGGTGGTGGGCGGCGTG
CTGGCCTGCTACAGCCTGCTGGTGACCGTGGCCTTCATCATCTTCTGGGTGCTGTGCGCCAGGCCCAGGAGGAGCCC
CGCCCAGGAGGACGGCAAGGTGTACATCAACATGCCCGGCAGGGGCAGGGTGAAGTTCAGCAGGAGCGCCGACGCCC
CCGCCTACCAGCAGGGCCAGAACCAGCTGTACAACGAGCTGAACCTGGGCAGGAGGGAGGAGTACGACGTGCTGGAC
AAGAGGAGGGGCAGGGACCCCGAGATGGGCGGCAAGCCCCAGAGGAGGAAGAACCCCCAGGAGGGCCTGTACAACGA
GCTGCAGAAGGACAAGATGGCCGAGGCCTACAGCGAGATCGGCATGAAGGGCGAGAGGAGGAGGGGCAAGGGCCACG
ACGGCCTGTACCAGGGCCTGAGCACCGCCACCAAGGACACCTACGACGCCCTGCACATGCAGGCCCTGCCCCCCAGG
TGATAACGCGTCATCATCGATCCGGATTAGGATCGATCCGTCCAATTTGTTAAAGACAGGATATCAGTGGTCCAGGC
TCTAGTTTTGACTCAACAATATCACCAGCTGAAGCCTATAGAGTACGAGCCATAGATAAAATAAAAGATTTTATTTA
GTCTCCAGAAAAAGGGGGGAATGAAAGACCCCACCTGTAGGTTTGGCAAGCTAGCTTAAGTAACGCCATTTTGCAAG
GCATGGAAAAATACATAACTGAGAATAGAGAAGTTCAGATCAAGGTCAGGAACAGATGGAACAGCTGAATATGGGCC
AAACAGGATATCTGTGGTAAGCAGTTCCTGCCCCGGCTCAGGGCCAAGAACAGATGGAACAGCTGAATATGGGCCAA
ACAGGATATCTGTGGTAAGCAGTTCCTGCCCCGGCTCAGGGCCAAGAACAGATGGTCCCCAGATGCGGTCCAGCCCT
CAGCAGTTTCTAGAGAACCATCAGATGTTTCCAGGGTGCCCCAAGGACCTGAAATGACCCTGTGCCTTATTTGAACT
AACCAATCAGTTCGCTTCTCGCTTCTGTTCGCGCGCTTCTGCTCCCCGAGCTCAATAAAAGAGCCCACAACCCCTCA
CTCGGGGCGCCAGTCCTCCGATTGACTGAGTCGCCCGGGTACCCGTGTATCCAATAAACCCTCTTGCAGTTGCATCC
GACTTGTGGTCTCGCTGTTCCTTGGGAGGGTCTCCTCTGAGTGATTGACTACCCGTCAGCGGGGGTCTTTCACACAT
GCAGCATGTATCAAAATTAATTTGGTTTTTTTTCTTAAGTATTTACATTAAATGGCCATAGTACTTAAAGTTACATT
GGCTTCCTTGAAATAAACATGGAGTATTCAGAATGTGTCATAAATATTTCTAATTTTAAGATAGTATCTCCATTGGC
TTTCTACTTTTTCTTTTATTTTTTTTTGTCCTCTGTCTTCCATTTGTTGTTGTTGTTGTTTGTTTGTTTGTTTGTTG
GTTGGTTGGTTAATTTTTTTTTAAAGATCCTACACTATAGTTCAAGCTAGACTATTAGCTACTCTGTAACCCAGGGT
GACCTTGAAGTCATGGGTAGCCTGCTGTTTTAGCCTTCCCACATCTAAGATTACAGGTATGAGCTATCATTTTTGGT
ATATTGATTGATTGATTGATTGATGTGTGTGTGTGTGATTGTGTTTGTGTGTGTGACTGTGAAAATGTGTGTATGGG
TGTGTGTGAATGTGTGTATGTATGTGTGTGTGTGAGTGTGTGTGTGTGTGTGTGCATGTGTGTGTGTGTGACTGTGT
CTATGTGTATGACTGTGTGTGTGTGTGTGTGTGTGTGTGTGTGTGTGTGTGTGTGTGTGTTGTGAAAAAATATTCTA
TGGTAGTGAGAGCCAACGCTCCGGCTCAGGTGTCAGGTTGGTTTTTGAGACAGAGTCTTTCACTTAGCTTGGAATTC
ACTGGCCGTCGTTTTACAACGTCGTGACTGGGAAAACCCTGGCGTTACCCAACTTAATCGCCTTGCAGCACATCCCC
CTTTCGCCAGCTGGCGTAATAGCGAAGAGGCCCGCACCGATCGCCCTTCCCAACAGTTGCGCAGCCTGAATGGCGAA
TGGCGCCTGATGCGGTATTTTCTCCTTACGCATCTGTGCGGTATTTCACACCGCATATGGTGCACTCTCAGTACAAT
CTGCTCTGATGCCGCATAGTTAAGCCAGCCCCGACACCCGCCAACACCCGCTGACGCGCCCTGACGGGCTTGTCTGC
TCCCGGCATCCGCTTACAGACAAGCTGTGACCGTCTCCGGGAGCTGCATGTGTCAGAGGTTTTCACCGTCATCACCG
AAACGCGCGATGACGAAAGGGCCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTCTAAATACATTCAAATATGTAT
CCGCTCATGAGACAATAACCCTGATAAATGCTTCAATAATATTGAAAAAGGAAGAGTATGAGTATTCAACATTTCCG
TGTCGCCCTTATTCCCTTTTTTGCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACAGCGGTAAGATCCTTGAGAGTTTT
CGCCCCGAAGAACGTTTTCCAATGATGAGCACTTTTAAAGTTCTGCTATGTGGCGCGGTATTATCCCGTATTGACGC
CGGGCAAGAGCAACTCGGTCGCCGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATGGGGGATCATGTAACTCGCCTTGA
TCGTTGGGAACCGGAGCTGAATGAAGCCATACCAAACGACGAGCGTGACACCACGATGCCTGTAGCAATGGCAACAA
CGTTGCGCAAACTATTAACTGGCGAACTACTTACTCTAGCTTCCCGGCAACAATTAATAGACTGGATGGAGGCGGAT
AAAGTTGCAGGACCACTTCTGCGCTCGGCCCTTCCGGCTGGCTGGTTTATTGCTGATAAATCTGGAGCCGGTGAGCG
TGGGTCTCGCGGTATCATTGCAGCACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCACTGATTAAGCATTGGTAACTGTCA
GACCAAGTTTACTCATATATACTTTAGATTGATTTAAAACTTCATTTTTAATTTAAAAGGATCTAGGTGAAGATCCT
TTTTGATAATCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTCCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTTGGAGCGAACGACCTACACCGAACTGAG
ATACCTACAGCGTGAGCATTGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCTTTGCTCTTAGGAGTTTCCTAATACATCCCAAACTCAAATATATAAAGCATTTGACTTGTT
CTATGCCCTAGGGGGCGGGGGGAAGCTAAGCCAGCTTTTTTTAACATTTAAAATGTTAATTCCATTTTAAATGCACA
GATGTTTTTATTTCATAAGGGTTTCAATGTGCATGAATGCTGCAATATTCCTGTTACCAAAGCTAGTATAAATAAAA
ATAGATAAACGTGGAAATTACTTAGAGTTTCTGTCATTAACGTTTCCTTCCTCAGTTGACAACATAAATGCGCTGCT
GAGCAAGCCAGTTTGCATCTGTCAGGATCAATTTCCCATTATGCCAGTCATATTAATTACTAGTCAATTAGTTGATT
TTTATTTTTGACATATACATGTGATGAAAGACCCCACCTGTAGGTTTGGCAAGCTAGCTTAAGTAACGCCATTTTGC
AAGGCATGGAAAAATACATAACTGAGAATAGAAAAGTTCAGATCAAGGTCAGGAACAGATGGAACAGCTGAATATGG
GCCAAACAGGATATCTGTGGTAAGCAGTTCC
pKB0174 plasmid (SEQ ID NO: 220):
TGCCCCGGCTCAGGGCCAAGAACAGATGGAACAGCTGAATATGGGCCAAACAGGATATCTGTGGTAAGCAGTTCCTG
CCCCGGCTCAGGGCCAAGAACAGATGGTCCCCAGATGCGGTCCAGCCCTCAGCAGTTTCTAGAGAACCATCAGATGT
TTCCAGGGTGCCCCAAGGACCTGAAATGACCCTGTGCCTTATTTGAACTAACCAATCAGTTCGCTTCTCGCTTCTGT
TCGCGCGCTTATGCTCCCCGAGCTCAATAAAAGAGCCCACAACCCCTCACTCGGGGCGCCAGTCCTCCGATTGACTG
AGTCGCCCGGGTACCCGTGTATCCAATAAACCCTCTTGCAGTTGCATCCGACTTGTGGTCTCGCTGTTCCTTGGGAG
GGTCTCCTCTGAGTGATTGACTACCCGTCAGCGGGGGTCTTTCATTTGGGGGCTCGTCCGGGATCGGGAGACCCCTG
CCCAGGGACCACCGACCCACCACCGGGAGGTAAGCTGGCCAGCAACTTATCTGTGTCTGTCCGATTGTCTAGTGTCT
ATGACTGATTTTATGCGCCTGCGTCGGTACTAGTTAGCTAACTAGCTCTGTATCTGGCGGACCCGTGGTGGAACTGA
CGAGTTCGGAACACCCGGCCGCAACCCTGGGAGACGTCCCAGGGACTTCGGGGGCCGTTTTTGTGGCCCGACCTGAG
TCCTAAAATCCCGATCGTTTAGGACTCTTTGGTGCACCCCCCTTAGAGGAGGGATATGTGGTTCTGGTAGGAGACGA
GAACCTAAAACAGTTCCCGCCTCCGTCTGAATTTTTGCTTTCGGTTTGGGACCGAAGCCGCGCCGCGCGTCTTGTCT
GCTGCAGCATCGTTCTGTGTTGTCTCTGTCTGACTGTGTTTCTGTATTTGTCTGAAAATATGGGCCCGGGCTAGCCT
GTTACCACTCCCTTAAGTTTGACCTTAGGTCACTGGAAAGATGTCGAGCGGATCGCTCACAACCAGTCGGTAGATGT
CAAGAAGAGACGTTGGGTTACCTTCTGCTCTGCAGAATGGCCAACCTTTAACGTCGGATGGCCGCGAGACGGCACCT
TTAACCGAGACCTCATCACCCAGGTTAAGATCAAGGTCTTTTCACCTGGCCCGCATGGACACCCAGACCAGGTGGGG
TACATCGTGACCTGGGAAGCCTTGGCTTTTGACCCCCCTCCCTGGGTCAAGCCCTTTGTACACCCTAAGCCTCCGCC
TCCTCTTCCTCCATCCGCCCCGTCTCTCCCCCTTGAACCTCCTCGTTCGACCCCGCCTCGATCCTCCCTTTATCCAG
CCCTCACTCCTTCTCTAGGCGCCCCCATATGGCCATATGAGATCTTATATGGGGCACCCCCGCCCCTTGTAAACTTC
CCTGACCCTGACATGACAAGAGTTACTAACAGCCCCTCTCTCCAAGCTCACTTACAGGCTCTCTACTTAGTCCAGCA
CGAAGTCTGGAGACCTCTGGCGGCAGCCTACCAAGAACAACTGGACCGACCGGTGGTACCTCACCCTTACCGAGTCG
GCGACACAGTGTGGGTCCGCCGACACCAGACTAAGAACCTAGAACCTCGCTGGAAAGGACCTTACACAGTCCTGCTG
ACCACCCCCACCGCCCTCAAAGTAGACGGCATCGCAGCTTGGATACGACGCCGCCCACGTGAAGGCTGCCGACCCCG
GGGGTGGACCATCCTCTAGACTGCCAGCGGCCGCTCGAGCCACCATGGCTCTTCCGGTAACCGCTCTGCTGCTTCCT
TTGGCTCTGCTTCTTCACGCGGCTAGGCCAGGGAGCGAATTGCCTACGCAGGGGACTTTTTCAAATGTAAGCACGAA
TGTGAGCTCTCCGGGATCCCCAGGATGGTTCCTGGACAGCCCAGATCGGCCCTGGAATCCCCCTACCTTTTCCCCTG
CCCTGCTGGTGGTGACAGAGGGCGACAACGCCACCTTCACATGCAGCTTTTCCAACACCTCTGAGAGCTTCGTGCTG
AATTGGTACAGAATGTCCCCATCTAACCAGACAGATAAGCTGGCCGCATTTCCAGAGGACAGGTCCCAGCCTGGACA
GGATTGCCGCTTCCGGGTGACCCAGCTGCCCAATGGCAGAGACTTTCACATGAGCGTGGTGAGGGCCCGGAGAAACG
ATTCCGGCACATACCTGTGCGGAGCCATCTCTCTGGCCCCAAAGGCACAGATCAAGGAGAGCCTGAGGGCAGAGCTG
AGGGTGACCGAGAGGAGGGCAGAGGTGCCTACAGCACACCCAAGCCCTTCCCCAAGACCAGCAGGACAGTTCCAGAC
CCTGGTGTGCCCTTCCCCACTGTTCCCAGGACCTTCTAAGCCCTTTTGGGTGCTGGTGGTGGTGGGAGGCGTGCTGG
CCTGCTACTCTCTGCTGGTGACAGTGGCCTTCATCATCTTTTGGGTGCTGTGCGCCCGCCCTCGGAGAAGCCCAGCA
CAGGAGGACGGCAAGGTGTATATCAATATGCCAGGCAGAGGCAGGGTGAAGTTTTCTCGGAGCGCCGATGCACCAGC
ATACCAGCAGGGACAGAACCAGCTGTATAACGAGCTGAATCTGGGCAGGCGCGAGGAGTACGACGTGCTGGATAAGC
GGAGAGGCAGAGACCCAGAGATGGGAGGCAAGCCACAGAGGAGGAAGAACCCTCAGGAGGGCCTGTACAATGAGCTG
CAGAAGGACAAGATGGCCGAGGCCTATTCCGAGATCGGCATGAAGGGAGAGCGGAGAAGGGGCAAGGGACACGATGG
CCTGTACCAGGGCCTGTCTACCGCCACAAAGGACACCTATGATGCCCTGCACATGCAGGCCCTGCCACCCAGGTGAT
AACGCGTCATCATCGATCCGGATTAGGATCGATCCGTCCAATTTGTTAAAGACAGGATATCAGTGGTCCAGGCTCTA
GTTTTGACTCAACAATATCACCAGCTGAAGCCTATAGAGTACGAGCCATAGATAAAATAAAAGATTTTATTTAGTCT
CCAGAAAAAGGGGGGAATGAAAGACCCCACCTGTAGGTTTGGCAAGCTAGCTTAAGTAACGCCATTTTGCAAGGCAT
GGAAAAATACATAACTGAGAATAGAGAAGTTCAGATCAAGGTCAGGAACAGATGGAACAGCTGAATATGGGCCAAAC
AGGATATCTGTGGTAAGCAGTTCCTGCCCCGGCTCAGGGCCAAGAACAGATGGAACAGCTGAATATGGGCCAAACAG
GATATCTGTGGTAAGCAGTTCCTGCCCCGGCTCAGGGCCAAGAACAGATGGTCCCCAGATGCGGTCCAGCCCTCAGC
AGTTTCTAGAGAACCATCAGATGTTTCCAGGGTGCCCCAAGGACCTGAAATGACCCTGTGCCTTATTTGAACTAACC
AATCAGTTCGCTTCTCGCTTCTGTTCGCGCGCTTCTGCTCCCCGAGCTCAATAAAAGAGCCCACAACCCCTCACTCG
GGGCGCCAGTCCTCCGATTGACTGAGTCGCCCGGGTACCCGTGTATCCAATAAACCCTCTTGCAGTTGCATCCGACT
TGTGGTCTCGCTGTTCCTTGGGAGGGTCTCCTCTGAGTGATTGACTACCCGTCAGCGGGGGTCTTTCACACATGCAG
CATGTATCAAAATTAATTTGGTTTTTTTTCTTAAGTATTTACATTAAATGGCCATAGTACTTAAAGTTACATTGGCT
TCCTTGAAATAAACATGGAGTATTCAGAATGTGTCATAAATATTTCTAATTTTAAGATAGTATCTCCATTGGCTTTC
TACTTTTTCTTTTATTTTTTTTTGTCCTCTGTCTTCCATTTGTTGTTGTTGTTGTTTGTTTGTTTGTTTGTTGGTTG
GTTGGTTAATTTTTTTTTAAAGATCCTACACTATAGTTCAAGCTAGACTATTAGCTACTCTGTAACCCAGGGTGACC
TTGAAGTCATGGGTAGCCTGCTGTTTTAGCCTTCCCACATCTAAGATTACAGGTATGAGCTATCATTTTTGGTATAT
TGATTGATTGATTGATTGATGTGTGTGTGTGTGATTGTGTTTGTGTGTGTGACTGTGAAAATGTGTGTATGGGTGTG
TGTGAATGTGTGTATGTATGTGTGTGTGTGAGTGTGTGTGTGTGTGTGTGCATGTGTGTGTGTGTGACTGTGTCTAT
GTGTATGACTGTGTGTGTGTGTGTGTGTGTGTGTGTGTGTGTGTGTGTGTGTGTGTTGTGAAAAAATATTCTATGGT
AGTGAGAGCCAACGCTCCGGCTCAGGTGTCAGGTTGGTTTTTGAGACAGAGTCTTTCACTTAGCTTGGAATTCACTG
GCCGTCGTTTTACAACGTCGTGACTGGGAAAACCCTGGCGTTACCCAACTTAATCGCCTTGCAGCACATCCCCCTTT
CGCCAGCTGGCGTAATAGCGAAGAGGCCCGCACCGATCGCCCTTCCCAACAGTTGCGCAGCCTGAATGGCGAATGGC
GCCTGATGCGGTATTTTCTCCTTACGCATCTGTGCGGTATTTCACACCGCATATGGTGCACTCTCAGTACAATCTGC
TCTGATGCCGCATAGTTAAGCCAGCCCCGACACCCGCCAACACCCGCTGACGCGCCCTGACGGGCTTGTCTGCTCCC
GGCATCCGCTTACAGACAAGCTGTGACCGTCTCCGGGAGCTGCATGTGTCAGAGGTTTTCACCGTCATCACCGAAAC
GCGCGATGACGAAAGGGCCTCGTGATACGCCTATTTTTATAGGTTAATGTCATGATAATAATGGTTTCTTAGACGTC
AGGTGGCACTTTTCGGGGAAATGTGCGCGGAACCCCTATTTGTTTATTTTTCTAAATACATTCAAATATGTATCCGC
TCATGAGACAATAACCCTGATAAATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAACGCTGGTGAAAGTAAAAGATGC
TGAAGATCAGTTGGGTGCACGAGTGGGTTACATCGAACTGGATCTCAACAGCGGTAAGATCCTTGAGAGTTTTCGCC
CCGAAGAACGTTTTCCAATGATGAGCACTTTTAAAGTTCTGCTATGTGGCGCGGTATTATCCCGTATTGACGCCGGG
CAAGAGCAACTCGGTCGCCGCATACACTATTCTCAGAATGACTTGGTTGAGTACTCACCAGTCACAGAAAAGCATCT
TACGGATGGCATGACAGTAAGAGAATTATGCAGTGCTGCCATAACCATGAGTGATAACACTGCGGCCAACTTACTTC
TGACAACGATCGGAGGACCGAAGGAGCTAACCGCTTTTTTGCACAACATGGGGGATCATGTAACTCGCCTTGATCGT
TGGGAACCGGAGCTGAATGAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGATTAAGCATTGGTAACTGTCAGACC
AAGTTTACTCATATATACTTTAGATTGATTTAAAACTTCATTTTTAATTTAAAAGGATCTAGGTGAAGATCCTTTTT
GATAATCTCATGACCAAAATCCCTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAACCACCGCTACCAGCGGTGGTTT
GTTTGCCGGATCAAGAGCTACCAACTCTTTTTCCGAAGGTAACTGGCTTCAGCAGAGCGCAGATACCAAATACTGTC
CTTCTAGTGTAGCCGTAGTTAGGCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAACGACCTACACCGAACTGAGATAC
CTACAGCGTGAGCATTGAGAAAGCGCCACGCTTCCCGAAGGGAGAAAGGCGGACAGGTATCCGGTAAGCGGCAGGGT
CGGAACAGGAGAGCGCACGAGGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAAAACGCCAGCAACGCGGCCTTT
TTACGGTTCCTGGCCTTTTGCTGGCCTTTTGCTCACATGTTCTTTCCTGCGTTATCCCCTGATTCTGTGGATAACCG
TATTACCGCCTTTGAGTGAGCTGATACCGCTCGCCGCAGCCGAACGACCGAGCGCAGCGAGTCAGTGAGCGAGGAAG
CGGAAGAGCGCCCAATACGCAAACCGCCTCTCCCCGCGCGTTGGCCGATTCATTAATGCAGCTGGCACGACAGGTTT
CCCGACTGGAAAGCGGGCAGTGAGCGCAACGCAATTAATGTGAGTTAGCTCACTCATTAGGCACCCCAGGCTTTACA
CTTTATGCTTCCGGCTCGTATGTTGTGTGGAATTGTGAGCGGATAACAATTTCACACAGGAAACAGCTATGACCATG
ATTACGCCAAGCTTTGCTCTTAGGAGTTTCCTAATACATCCCAAACTCAAATATATAAAGCATTTGACTTGTTCTAT
GCCCTAGGGGGCGGGGGGAAGCTAAGCCAGCTTTTTTTAACATTTAAAATGTTAATTCCATTTTAAATGCACAGATG
TTTTTATTTCATAAGGGTTTCAATGTGCATGAATGCTGCAATATTCCTGTTACCAAAGCTAGTATAAATAAAAATAG
ATAAACGTGGAAATTACTTAGAGTTTCTGTCATTAACGTTTCCTTCCTCAGTTGACAACATAAATGCGCTGCTGAGC
AAGCCAGTTTGCATCTGTCAGGATCAATTTCCCATTATGCCAGTCATATTAATTACTAGTCAATTAGTTGATTTTTA
TTTTTGACATATACATGTGATGAAAGACCCCACCTGTAGGTTTGGCAAGCTAGCTTAAGTAACGCCATTTTGCAAGG
CATGGAAAAATACATAACTGAGAATAGAAAAGTTCAGATCAAGGTCAGGAACAGATGGAACAGCTGAATATGGGCCA
AACAGGATATCTGTGGTAAGCAGTTCC

### EXAMPLES

### Example 1: Peripheral Blood Mononuclear Cell (PBMC) Isolation

Peripheral blood mononuclear cells (PBMCs) were separated from the Buffy Coat leukopak blood product (Gulf Coast Blood Center, Houston, TX) prior to culture initiation to reduce red blood cell and platelet contamination within the initial cell population. Buffy coats product was diluted with an equal volume of 1×PBS and subjected to density gradient centrifugation in Ficoll-Paque medium according to manufacturer's instruction. The harvested PBMCs were resuspended in 45 ml OpTmizer media (Gibco) and the cell number was quantified and cell viability determined.

### Example 2: Day 0—Culture Initiation

Cells harvested as described in Example 1 were transferred to 750-AC bags (750×10⁶ cells/bag) and brought up to a final volume of 750 mL/bag using Complete Media. IL-2, IL-7 and Zoledronic Acid were added to each bag at a final concentration of 300 IU/mL, 250 U/mL and 5 μM, respectively. The cells were incubated at 37° C. in 5% CO₂ for 2-3 days.

### Example 3: Day 3 & 4—Feeding

IL-2 and IL-7 were added at day 3 to a final concentration of 300 IU/mL and 250 U/mL, respectively, to further stimulate T-cell expansion. The cells were incubated at 37° C. in 5% CO₂ for 3 days

### Example 4: Day 6—TCR αβ Depletion

Following 6 days of culture, TCR α/β⁺ T-cells were removed by paramagnetic bead labeling in conjunction with magnetic depletion on a CliniMACs plus device (Miltenyi). Cells from Example 3 were pelleted by centrifugation and resuspended in Running Buffer (PBS/EDTA supplemented with 0.5% (w/v) Human serum albumin (HSA)). The cells were stained with CliniMACs TCRα/β-Biotin reagent and then washed. The cells were resuspended in Running Buffer and stained with CliniMACs Anti-Biotin Reagent and then washed. The cells were then transferred to a cell preparation bag and connected to a CliniMACs depletion tubing set and the α/β⁺ T-cells were removed on the CliniMacs plus device. The cells not removed by the magnet (the γδ-T cell enriched fraction) were pelleted by centrifugation and washed. Cells were then resuspended in Complete Media with 300 U/ml IL-2 and 250 U/ml IL-7 to obtain a 1×10⁶ cells/ml suspension. This suspension was then transferred to a C bag and incubated at 37° C. in 5% CO₂ overnight.

### Example 5: Day 7—Cryopreservation of γδ T-cells

The cells from the culture enriched for γδ T-cells of Example 4 were harvested and cryopreserved on Day 7. Cells were pelleted by centrifugation and re-suspended in ice cold Cryostor CS5 at a concentration of 20×10⁶ cells/ml. Cells were then cryopreserved by being placed in in a controlled rate freezer (CRF) pre-cooled to 4° C. The freeze rate for cryopreservation was −1° C./min starting from 4° C. to until reaching −80° C. Cells were transferred in liquid nitrogen vapor phase until use.

### Example 6: Transduction of Culture Enriched for γδ T-cells

For cells enriched for γδ T-cells that are to be transduced, the cell culture of Example 4 was continued for 1-2 days (2-3 days total) and the cell culture C bags were coated with Retronectin to enhance retroviral-mediated gene transduction. Transduction was carried out be standard methods and the cells cryopreserved as described in Example 5.

### Example 7: Gamma Delta T-Cell Composition (KB-GDT-01 Drug Product (DP))

KB-GDT-01 is an allogeneic, “off-the-shelf”, gamma delta T cell therapy. Bulk Drug Substance is formulated, filled, finished, and cryopreserved as KB-GDT-01 Drug Product (DP).

KB-GDT-01 DP is formulated as a cell suspension at 2×10⁷ cells per mL in 10 mL CryoStor® CS5 and filled into CryoMACS® cryogenic freezing bags (Miltenyi Biotec). The CryoMACS® bags are indicated for cryopreservation applications at ultra-low temperatures (down to −196° C.) and marketed in the US under FDA 510(k) clearance. CryoStor® CS5 is a serum-free, protein-free, chemically defined cryopreservation medium designed to prepare and preserve cells in ultra-low temperature environments (−196° C. to −70° C.). It is pre-formulated with 5% dimethyl sulfoxide (DMSO) (v/v). The composition of KB-GDT-01 DP is summarized in Table 1.

TABLE 1
KB-GDT-01 Drug Product Composition

| | | Amount in | | Manufact- |
|---|---|---|---|---|
| | | KB-GDT-01 | | uring |
| | Manufac- | Drug | | Quality |
| Component | turer | Product | Function | Level |
| KB-GDT-01 | Kiromic | 2 × 10⁷ viable | Investigational | cGMP |
| DP | BioPharma | cells/mL | drug | |
| CryoStor ® | BioLife | 10 mL | Cryopreser- | cGMP |
| CS5 | Solutions | | vative | |

Abbreviations: cGMP = Current Good Manufacturing Practice, mL = milliliter

### Example 8: KB-GDT-01 DP Manufacturing Process

### Manufacturing Process Summary

KB-GDT-01 Drug Product (DP) is manufactured in an uninterrupted process from KB-GDT-01 Drug Substance (DS), a transient process intermediate, which is formulated, filled, finished, and cryopreserved as DP without the use of a hold step. Peripheral blood mononuclear cells (PBMCs) are isolated from fresh healthy donor leukapheresis, washed, and activated with zoledronic acid. Activated PBMCs are expanded in the presence of IL-2 and IL-7 then depleted of alpha beta T cells. The resulting cell product, which contains gamma delta T cells, is further expanded in media containing IL-2 and IL-7 before being formulated, filled, and finished into KB-GDT-01 drug product.

### Excipients

The excipient has been chosen to maintain cell viability during storage, shipment, and administration. KB-GDT-01 DP is formulated in CryoStor® CS5, a serum-free, protein-free, chemically defined cryopreservation medium designed to prepare and preserve cells in ultra-low temperature environments (−70° C. to −196° C.). It is pre-formulated with 5% dimethyl sulfoxide (DMSO) (v/v).

### Container

The final container closure for KB-GDT-01 consists of a CryoMACS© cryogenic freezing bag (Miltenyi Biotec).

### Description of Manufacturing Process

KB-GDT-01 is an allogeneic gamma delta T cell therapy. The manufacturing process for KB-GDT-01 Drug Substance is described herein.

Briefly, the source material for KB-GDT-01 is fresh healthy donor leukapheresis. The manufacture of KB-GDT-01 Drug Substance begins with isolating PBMCs from healthy donor leukapheresis and then washing the PBMCs. The PBMCs are activated with zoledronic acid and expanded in media containing IL-2 and IL-7 before undergoing depletion of alpha beta T cells. The resulting enriched gamma delta T cell product is further expanded in media containing IL-2 and IL-7 before being formulated, filled, and finished into KB-GDT-01 Drug Product (DP).

Fresh leukapheresis is used for KB-GDT-01 DS manufacture within 24 hours of collection.

Prior to processing, the leukapheresis is sampled for cell count, cell viability, and cell phenotype by flow cytometry assessment. Additionally, a sample is archived if sterility analysis is required in the case of an investigation.

On Day 0 of the manufacturing process, Peripheral blood mononuclear cells (PBMCs) are isolated from the leukapheresis by Ficoll density gradient centrifugation. The leukapheresis is diluted with DPBS without calcium chloride and magnesium chloride and then layered over Ficoll-Paque™PREMIUM. The suspension is centrifuged, and the lymphocyte layer is harvested. The harvested PBMCs are washed and concentrated by centrifugation. The supernatant is aspirated and the washed PBMCs are resuspended in Complete CTS™ Medium, which is comprised of OpTmizer CTS™ Basal Medium, OpTmizer CTS™ Supplement, Human AB Serum, and GlutaMAX™.

The washed PBMCs are resuspended in a Complete CTS™ Medium supplemented with Zoledronic Acid (in NaOH), Interleukin-2 (IL-2), and Interleukin-7 (IL-7). A sample is taken for a cell count before the cells are transferred to Vuelife 750-AC Cell Culture Bags (CCBs) at a fixed cell density and cultured for three days.

On Day 3 of the manufacturing process, the cultured PBMCs are replenished with Complete CTS™ Medium supplemented with IL-2 and IL-7 and without bisphosphonate (e.g. Zoledronic acid) supplementation.

On Day 6 of the manufacturing process, the media replenished PBMCs are pooled and depleted. The pooled cells are centrifuged and resuspended in CliniMACS® PBS/EDTA Buffer supplemented with Human Serum Albumin. Alpha beta T cell depletion is performed using a two-step labelling process with reagents from the CliniMACS® TCRα/β Kit:
1) The cells are incubated with TCRa/b Biotin for 30 minutes at room temperature. The cells are centrifuged, washed, and resuspended in CliniMACS© PBS/EDTA Buffer supplemented with Human Serum Albumin.
2) The washed cells are incubated and labeled with Anti-Biotin Reagent for 30 minutes at room temperature.

The labeled cells are centrifuged, resuspended, and washed with CliniMACS© PBS/EDTA Buffer supplemented with Human Serum Albumin prior to transfer to a cell preparation bag and welded to a CliniMACS© Depletion Tubing Set. The CliniMACS© Depletion Tubing Set is installed on the CliniMACS® Plus, where the alpha beta T-cells are separated from the starting population using a pre-defined program.

Following alpha beta T cell depletion, the target cells (gamma delta T cells) are resuspended in Complete CTS™ Medium with IL-2 and IL-7 and transferred to Vuelife 750-C CCBs at a set cell density for further expansion.

On Day 8, the expanded gamma delta T cells are pooled and harvested, created KB-GDT-01 Bulk Drug Substance. Cells from the KB-GDT-01 Bulk Drug Substance are sampled for mycoplasma testing.

### Example 9: Development of Formulation, Fill, Finish, and Cryopreservation to Generate KB-GDT-01 DP

A series of development runs were performed (DEV1-DEV7) with the primary goals to:
Optimize the length of culture before cryopreservation: day 8 versus day 9 from start of culture;
Optimize the cell density for cryopreservation; and
Optimize cryopreservation media (cryomedia).

Gamma Delta T cells are a viable alternative to traditional immunotherapies utilizing alpha beta T cells due to their independence from MHC-class restriction or neoantigen burden while exhibiting biophysical properties similar to natural killer (NK) cells with potent antitumor activity. Activation and expansion of gdT cells has been studied in academic settings but upscale and manufacture in GMP settings has not been widely developed.

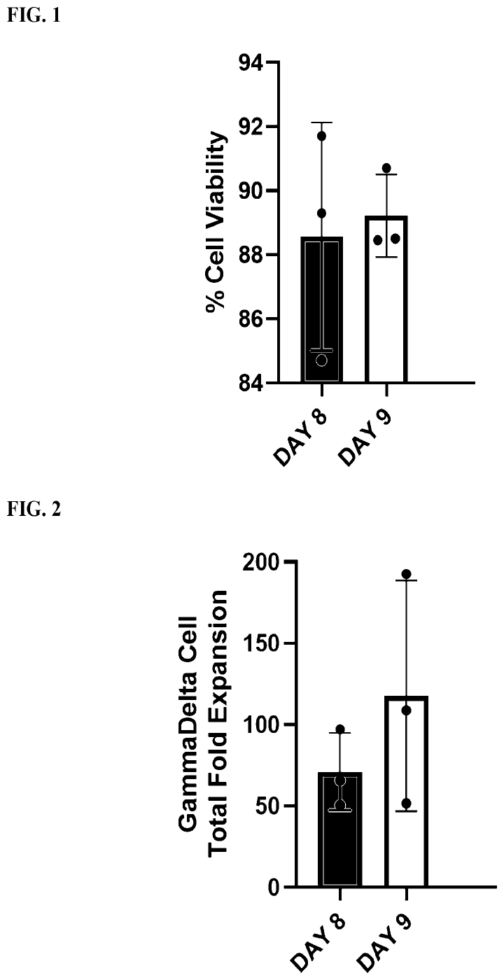

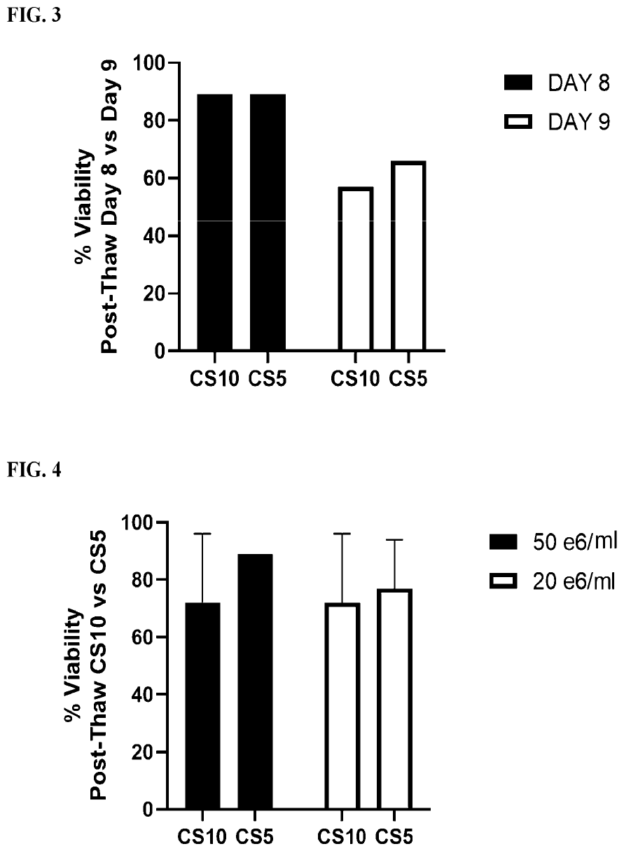

Briefly, fresh leukapheresis was obtained from healthy donors and mononuclear cells were isolated and cultured in a culture media containing zoledronic acid, IL2 and IL7 for about 1 to about 3 days to form a monocyte-depleted cell culture. Then, the cells were cultured in a culture media containing IL2 and IL7 and without zoledronic acid for about 1 to about 3 days. At Day 6 alpha beta (ab) T-cells were depleted and the remaining cells were cultured in a culture media containing IL2 and IL7 and without zoledronic acid for at least 1 day to form a gamma delta T-cell enriched culture. The cells were cultured using this protocol for either 8 or 9 days. While the viability of the culture expanded to either Day 8 or 9 did not exhibit any significant differences (FIG. 1—Day8: 88.6, Day 9: 89.2), the total fold expansion was more pronounced at Day 9 (FIG. 2—117.6 (day9) vs 71.1 (day8)). With the goal of generating an “off-the-shelf” cryopreserved product, we tested the viability post-thaw of the cell product cryopreserved in either CS10 or CS5, 2 different commercially available cryopreservation media (FIG. 3). At Day 9 there was a decrease in total cell viability in either cryopreservation media, suggesting that while more cells could be generated ex vivo through 9 days culture, Day 8 harvest appeared to yield optimal viable product.

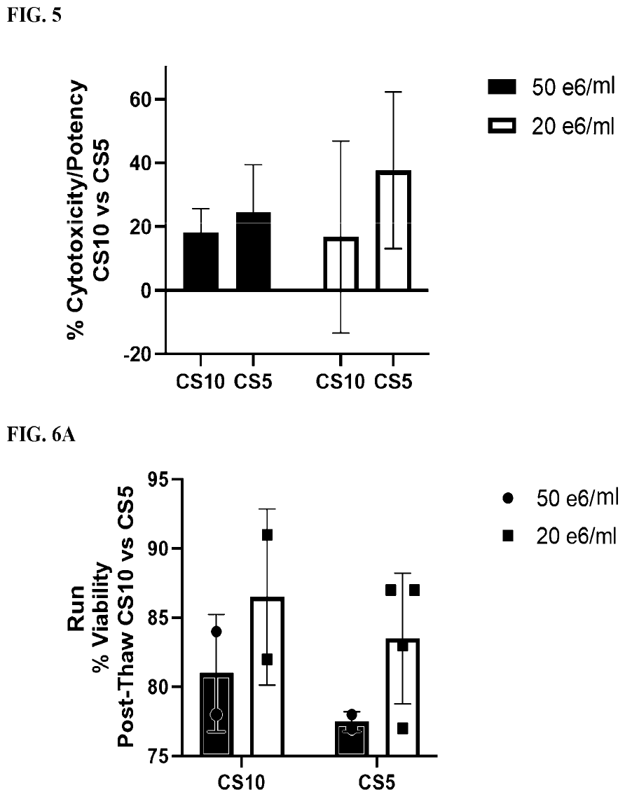

We tested cryopreservation of Day 8 ex vivo expanded gdT cells in either CS10 or CS5 and at 20e6 cells/ml and 50e6 cells/ml concentrations. gdT cells cryopreserved in CS5 demonstrated higher post-thaw viability at both 50e6/ml and 20e6/ml cell concentrations, compared to CS10 (FIG. 4). Additionally, in post-thaw functionality testing utilizing a cytotoxicity co-culture targeting SKOV3 ovarian cancer cells, CS5 cryopreserved gdT cells were more efficient than CS10 cryopreserved cells in promoting cytotoxicity of SKOV3 cells (FIG. 5). Cancer cell cytotoxicity was more pronounced at cell concentrations of 20e6 cells/ml compared to 50e6 cells/ml.

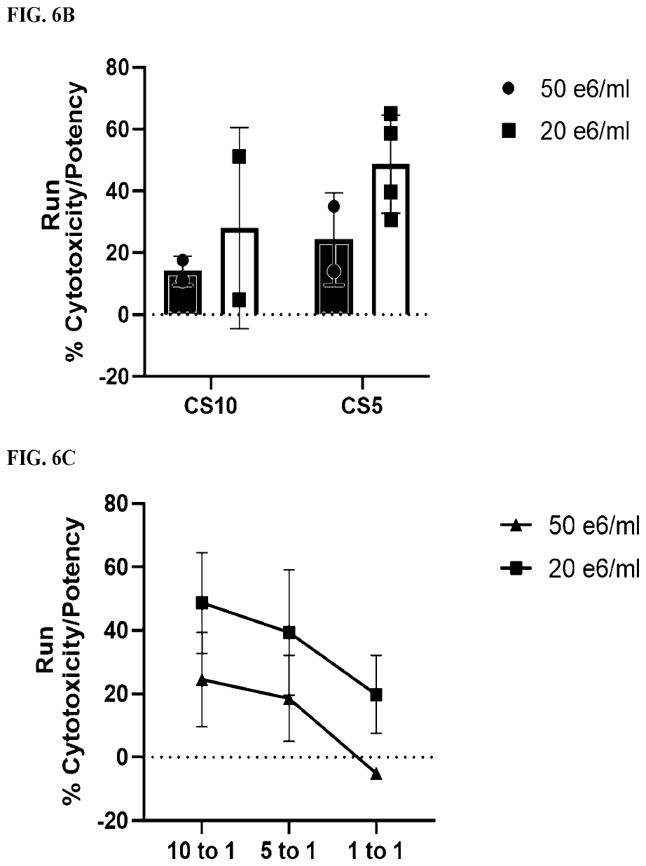

Finally, CS5 cryopreserved gdT cells at 20e6 cells/ml demonstrated higher viability (FIG. 6A) and cytotoxicity (FIG. 6B). CS5 cryopreserved gdT cells also maintained higher potency levels at 10:1, 5:1 and 1:1 ratios at 20e6 cells/ml.

### Example 10: Materials and Methods for Experiments with Gamma Delta T Cells Engineered with an Anti-PD-1 CAR

Cell Lines. Human cancer cell lines HCC827 (lung adenocarcinoma), NCI-H226 (mesothelioma), SKOV3 (ovarian) and normal healthy cells BEAS-2B (lung epithelial) and Hs888Lu (lung fibroblasts) were purchased from American Type Culture Collection (ATCC, Manassas, VA). PBMCs were purchased from ZenBio (Durham, NC). Cells were grown in complete RMPI (HCC827, NCI-H226) or DMEM (SKOV3, Hs888Lu, B16, ID8) media supplemented with 10% heat-inactivated fetal bovine serum, 100 U/ml penicillin, 100 g/ml streptomycin, 1 mM pyruvate, 10 mM Hepes, and 0.1 mM non-essential amino acids. BEAS-2B cells were grown in BEBM Bronchial Epithelial Cell Growth Basal Medium from Lonza.

Primary cells. Human bone marrow mononuclear cells, human skeletal muscle cells, human peripheral blood mononuclear cells, pooled donor human hepatocytes (20 donors), normal human astrocytes, human peripheral blood CD14+ monocytes, human CD19+ B cells, human intestinal epithelial cells, and human intestinal myofibroblasts were purchased from Lonza (Walkersville, MD, USA). Human bone marrow mononuclear cells and human peripheral blood CD14+ monocytes were grown in X-VIVO 15 media with L-Glutamine, gentamicin and phenol red. Human peripheral blood mononuclear cells and human CD19+ B cells were grown in X-VIVO 20 media with L-Glutamine, gentamicin and phenol red. Human skeletal muscle cells were grown in Skeletal Muscle Growth Medium. Pooled donor human hepatocytes were grown in Hepatocyte Culture Medium. Normal human astrocytes were grown in Astrocyte Growth Medium from Lonza. Human intestinal epithelial and myofibroblast cells were grown in Smooth Muscle Cell Growth Medium-2. Media and supplements were purchased from Lonza.

Generation of human chPD1-DAP10 receptor and retroviral supernatants. The human chPD1-DAP10 receptor consists of, from N- to C-terminus: extracellular region of human PD1, CD28 juxta-membrane region, CD28 transmembrane domain, DAP10 cytoplasmatic region, CD3z cytoplasmatic region. The retroviral vector is based on a Recombinant Moloney murine leukemia virus (MMLV), which uses the RD114 envelope protein. The system consists of a 3-plasmid transfection: one for the gag-pol protein, one for the envelope protein, and one containing the gene of interest, i.e., SFG. The chPD1-DAP10 sequence was inserted into the SFG vector under control of the LTR promoter. All sequence was verified by DNA sequencing. To generate retrovirus particles, Lenti-X 293T cells were maintained in DMEM+10% FBS, transfected with TransIT-LT1 transfection reagent (Mirus) mixed with pEQ-Pam3 package plasmid, RD114 package plasmid and SFG plus CAR plasmid. Virus supernatant were harvested at 48 h and 72 h post transfection, filtered with 45 uM filter and were frozen at −80° C. for later use.

Expression of chPD1-DAP10 receptor in human γδT cells. Human Gamma Delta T cells (gd-T) were isolated from cryopreserved human normal peripheral blood mononuclear cells (PBMCs) (ZenBio). The human PBMCs were extracted from whole blood using ficoll and were stored at −80 C until use. Human GDT were retrovirally transduced to express the chPD1-DAP10 receptor and non-transduced cells acted as a control. Briefly, freshly isolated PBMCs were cultured in CTS™ OpTmizer™ T Cell Expansion with 2% heat-inactivated human serum and 1% pen-strep and 300 IU/mL human IL-2 (Miltenyi Biotec) and 5 M zoledronic acid (Sigma). Every 2 to 3 days, cells were feed with fresh culture medium containing human IL-2 300 IU/ml. On day 7, cells were harvested and resuspended in medium. γδ-T cells were further purified using the γδ-T cells isolation kit (Miltenyi Biotec) following the manufacture's protocol. Purified γδ-T cells were subjected to retrovirus transduction or continue expansion with human IL-2 300 IU/ml for additional 7-14 days. On the day before the transduction, retronectin (Takara Bio) was coated onto 6-well non-TC treated plates at 10 μg/ml and incubated at 4° C. overnight. The next day, the retronectin solution was removed, the plate was rinsed with 2 ml sterile phosphate-buffered saline (PBS) and 5 ml of frozen CAR retrovirus supernatants were added into the retronectin-coated plate. The viral supernatants were spun at 2000×g for 2 h at 32° C. The viral supernatant was removed, the plate was washed with fresh medium and 2 million gd-T cells that were purified on day 7 culture were resuspended in CTS medium with 5% human serum, P/S, and 300 IU/ml hIL-2 were added into each well. The cells were spun at 1000×g for 10 min at 32° C. and were cultured at 37° C. for additional 7-14 days. Fresh media with hIL-2 was added every 2-3 days. The transduction efficiency was determined on day 3-7 post transduction based on the detection of PD-1.

Flow cytometry. The purity of γδT cells and the expression of PD1 on T cells was tested using flow cytometry. Cells were stained with FITC-labelled anti-CD3 (clone UCHT1), PE-labelled anti-PD1 (clone RMP1-30), PerCP-labelled anti-Vδ2 antibodies or isotype controls. All antibodies were purchased from BioLegend. Cell fluorescence was measured using an Accuri C6 flow cytometer.

Cytotoxicity by chPD1-DAP10 γST cells. To determine lysis of tumor cells, chPD1 or non-transduced T cells (1×10) were cultured with tumor cells at various effector to target ratios (E:T 25:1, 5:1, 4:1, 1:1, 0.5:1, 0.25:1). Specific lysis was measured using an LDH cytotoxicity assay kit (Pierce) according to manufacturer's instructions. In some experiments, a more sensitive flow-cytometry based method was used to detect target cell death. Target cells were labelled with CFSE prior to incubation with T cells. Viable and dead cells were discriminated using the eFluor™ 450 Viability Dye (eBioscience), and the number of CFSE+, eFluor-negative cells was determined in the CD3-negative gate.

### Example 11: Gamma Delta T Cells Engineered with a Chimeric PD-1 Receptor Effectively Control PD-L1 Positive Tumors In Vitro and In Vivo with Minimal Toxicities

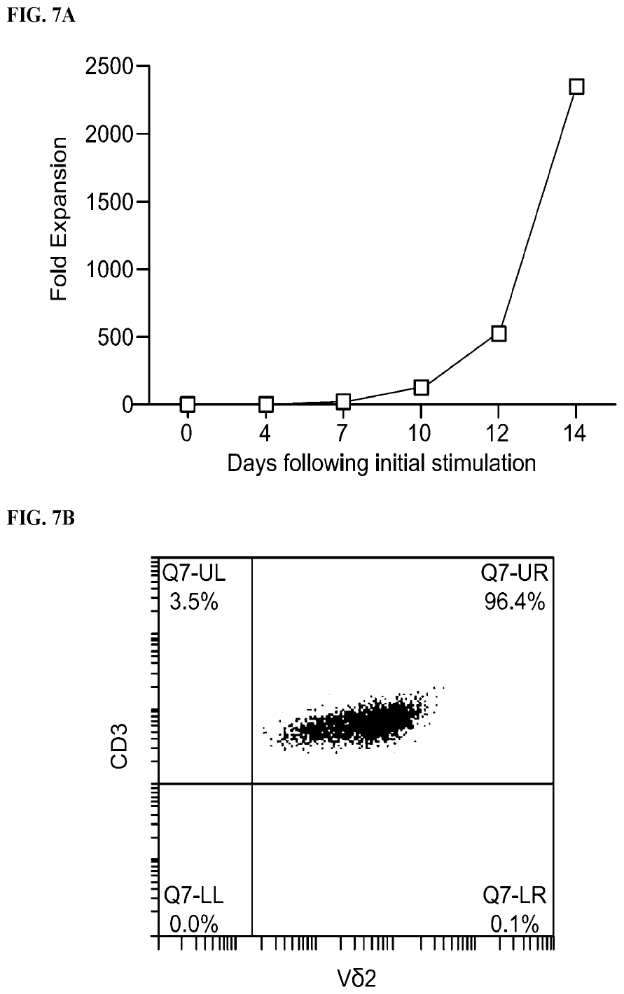

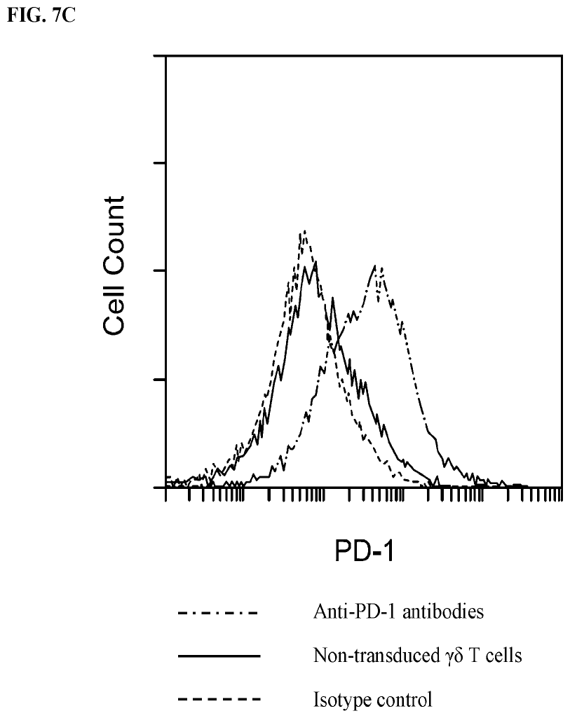

Transduced human gamma delta T cells express chPD1 receptor and expand in vitro. Previously, a chPD1 receptor consisting of the extracellular region of the PD1 receptor fused with the intracellular regions of the DAP10 costimulatory receptor and CD3ζ was created and murine αβT cells expressing the chPD1-DAP10 receptor decreased tumor burden and increased survival in multiple models of cancer. However, the anti-tumor activity of a human chPD1-DAP10 receptor expressed in γδT cells is still unknown. Therefore, human γδT cells were isolated from cryopreserved PBMCs and were retrovirally transduced to express the chPD1-DAP10 receptor. Similar to non-transduced T cells, transduced chPD1-DAP10 γδT cells expanded more than 2,000-fold over a 14-day period in vitro (FIG. 7A). After in vitro expansion, the chPD1-DAP10 T cell population consisted of 99.9% CD3+ cells and >95% Vδ2+ and Vγ9+ cells (FIG. 7B). Seven days after transduction, chPD1-DAP10 γδT cells had increased cell surface expression of PD1 compared to non-transduced γδT cells, indicating that the chPD1-DAP10 receptor was expressed on the γδT cells (FIG. 7C). These data demonstrate that the human chPD1-DAP10 receptor can be expressed on PBMC-derived γδT cells and that expression of this receptor does not alter γδT cell expansion in vitro.

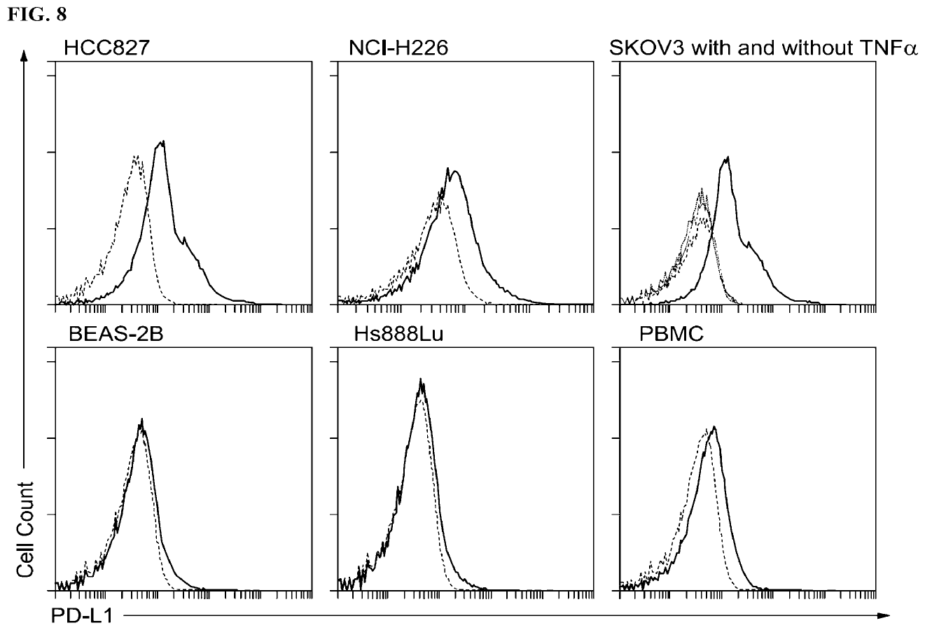

Human gamma delta chPD1-expressing T cells lyse PD-L1-expressing tumor cells. ChPD1-DAP10 γδT cell responses against human tumor cell lines was determined. HCC827 (human lung adenocarcinoma) and NCI-H226 (human mesothelioma) cell lines expressed cell surface PD-L1 as determined by flow cytometry (FIG. 8). The SKOV3 (human ovarian cancer) cell line did not express high levels of PD-L1 however incubation of SKOV3 cells with 50 pg/mL TNFα increased cell surface expression of PD-L1 after 48 hours. Compared to human cancer cell lines, human lung epithelial (BEAS-2B) cells, human lung fibroblasts (Hs888Lu), and human PBMCs did not have high expression of cell surface PD-L1. These data suggest that these PD-L1-positive human cancer cell lines, but not normal human tissues, may be potential targets for chPD1-DAP10 γST cells.

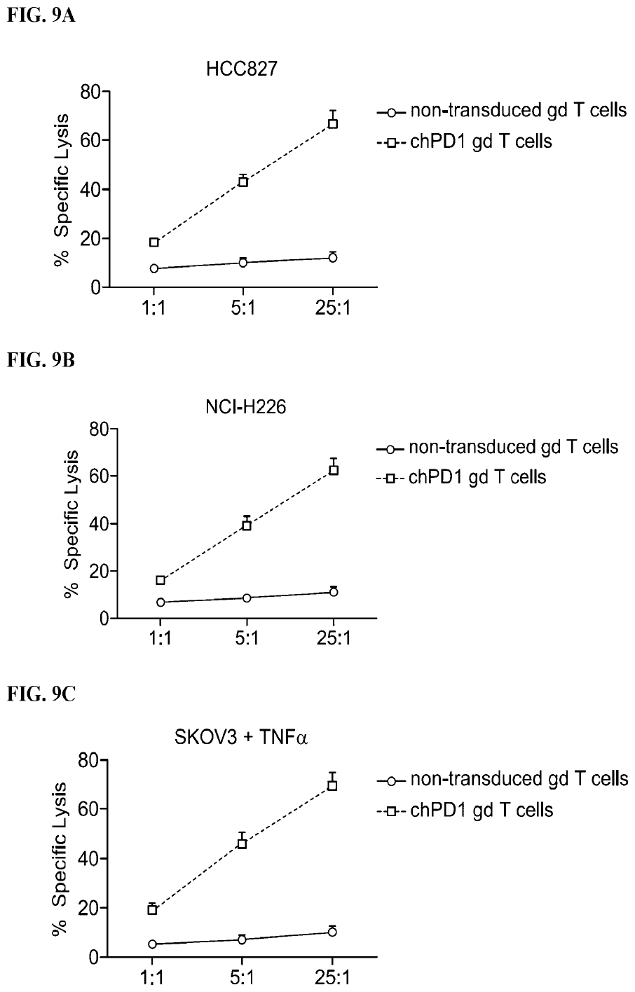

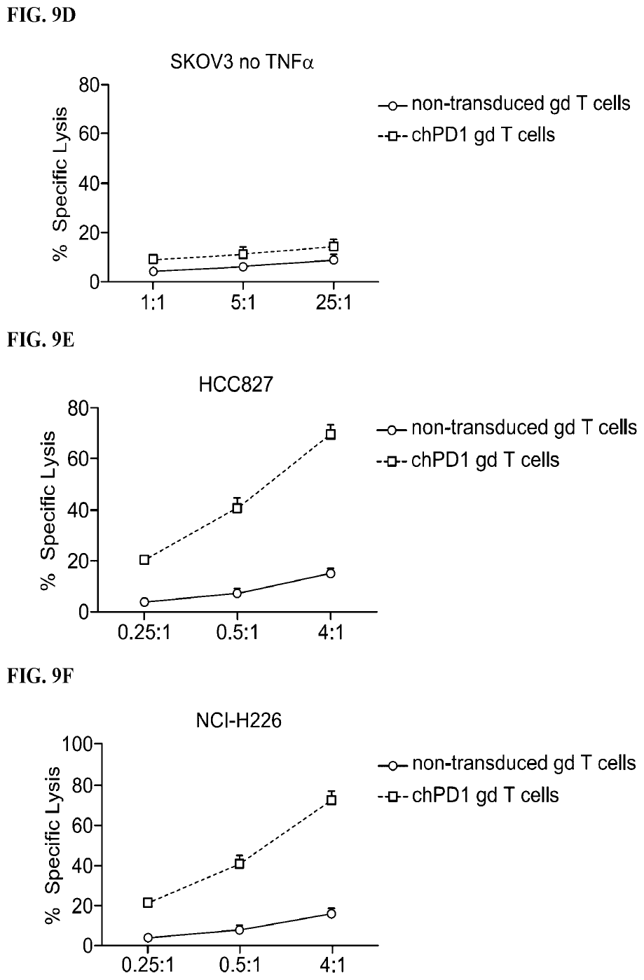

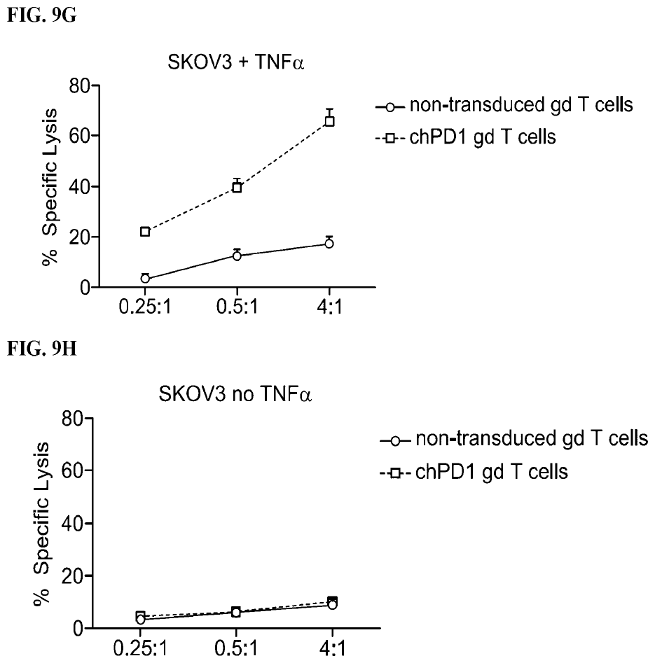

Compared to non-transduced γδT cells, chPD1-DAP10 γδT cells demonstrated significant lysis of PD-L1-positive tumor cells HCC827 and NCI-H226 (FIGS. 9A-9B and 9E-9F). ChPD1-DAP10 γST cells also lysed SKOV3 cells that were preincubated with TNFα (FIGS. 9C and 9G) but did lyse SKOV3 cells without TNFα pre-treatment (FIGS. 9D and 9H). Furthermore, chPD1-DAP10 γδT cells did not show significant lysis of human lung epithelial cells, lung fibroblasts, allogeneic PBMCs, or autologous PBMCs (and data not shown). This demonstrates that chPD1-DAP10 γδT cells lyse PD-L1 positive cells but do not lyse cells that have low or no cell surface expression of PD-L1.

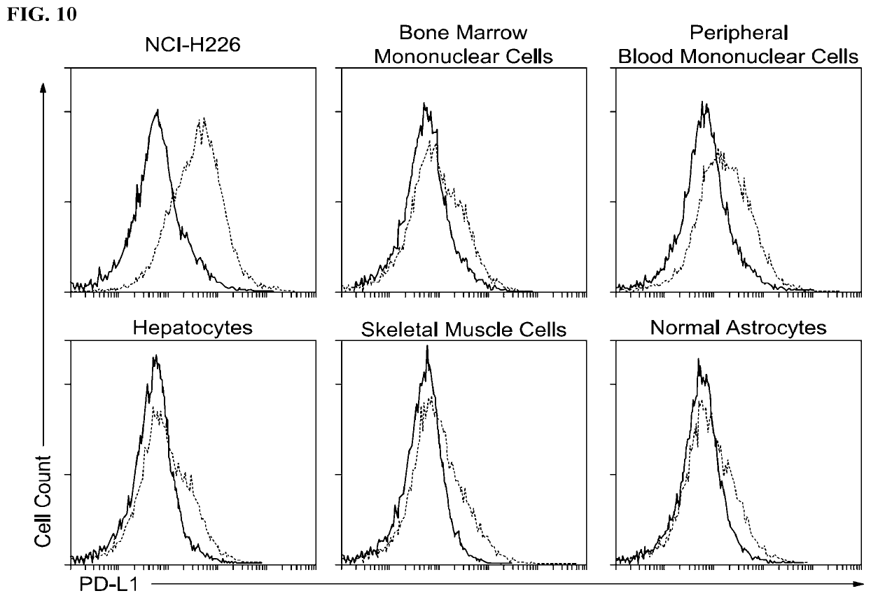

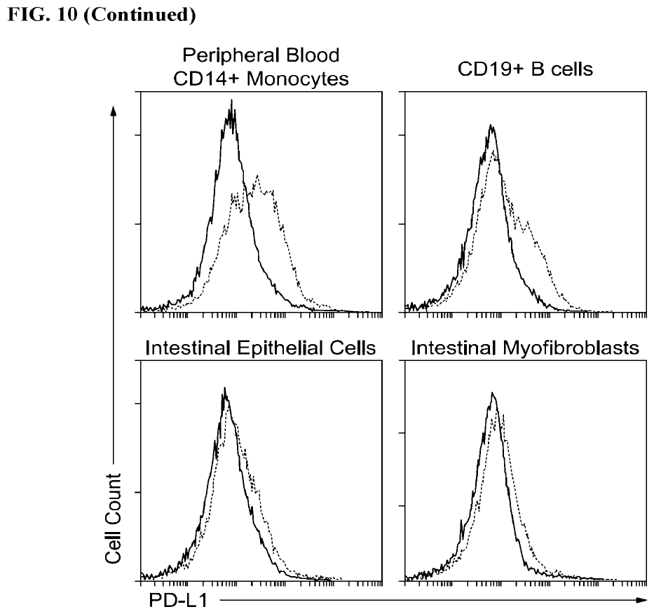

Primary cells from healthy tissues express variable PD-L1 levels on the membrane surface. Expression of PD-L1 was evaluated on the human cancer cell line NCI-H226 (mesothelioma), and normal human healthy cells: human bone marrow mononuclear cells, human skeletal muscle cells, human peripheral blood mononuclear cells, pooled donor human hepatocytes, normal human astrocytes, human peripheral blood CD14+ monocytes, human CD19+ B cells, human intestinal epithelial, and human intestinal myofibroblast cells (FIG. 10). The NCI-H226 cell line expressed cell surface PD-L1 as determined by flow cytometry (FIG. 10). PD-L1 expression in normal healthy cells was variable with human bone marrow mononuclear cells, peripheral blood mononuclear cells, peripheral blood CD14+ monocytes, CD19+ B cells, hepatocytes, skeletal muscle cells, and normal astrocytes showing moderate cell surface expression of PD-L1 and intestinal epithelial cells and myofibroblasts showing low cell surface expression of PD-L1 (FIG. 10).

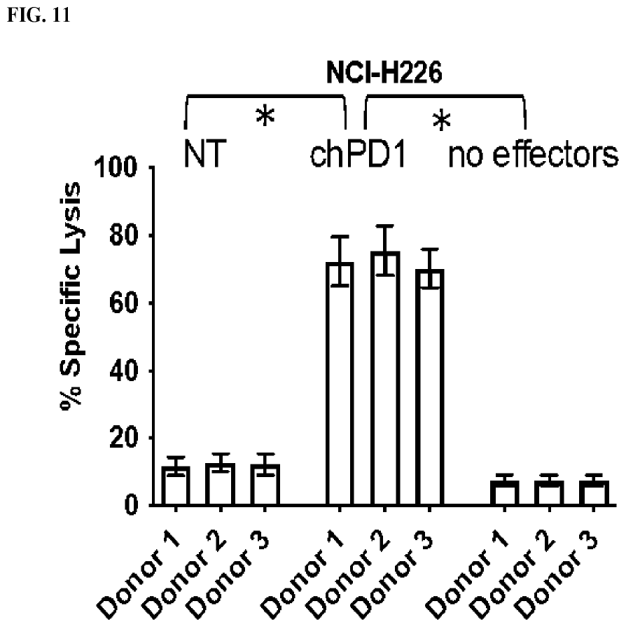

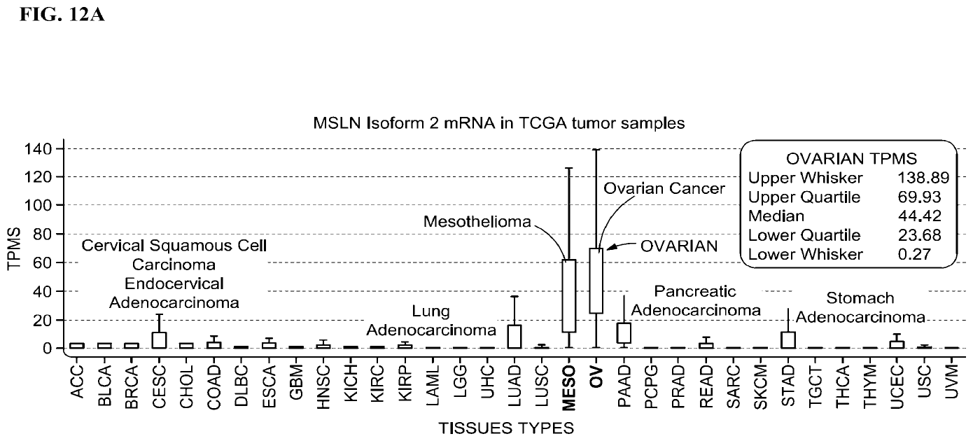

Human gamma delta chPD1-expressing T cells do not respond to cultured primary human cells from healthy tissues. chPD1-GDT demonstrated significant lysis of the PD-L1-positive NCI-H226 mesothelioma tumor cells while non-transduced GDT had a limited effect (FIG. 11). Furthermore, chPD1-GDT had no effect on the viability of healthy human cells that showed moderate cell surface expression of PD-L1 including human bone marrow mononuclear cells, peripheral blood mononuclear cells, peripheral blood CD14+ monocytes, CD19+ B cells, hepatocytes, skeletal muscle cells, and normal astrocytes or with low PD-L1 cell surface expression including human intestinal epithelial cells and human intestinal myofibroblasts. A total of three independent donors was assessed with generally similar results obtained for each as shown in FIG. 8. These data demonstrate that chPD1-GDT lyse PD-L1 tumor cells but do not show on-target/off-tumor cytotoxicity on healthy human cells.

### Discussion

Here, we tested the chPD1 receptor in unconventional T cells. Such cells present several advantages. Unconventional T cells, namely represented by γδT cells, are independent from HLA-peptide complexes, therefore allowing for allogenic administration, which means that such cells can be engineered and prepared “off-the-shelf”.

Our results show that peripheral blood γδT cells can be efficiently transduced ex vivo with a retroviral vector encoding the chPD1-DAP10-CD3z construct, without affecting viability or proliferation in vitro. The chPD1 receptor made γδT cells capable of responding to target cells in a PDL1-specific manner, which resulted in efficient cytotoxicity and in the production of Th1-type cytokines.

### Example 12: Materials and Methods for Experiments with Gamma Delta T Cells Engineered with an Anti-Mesothelin Isoform 2 CAR

Cell lines and culture medium. All parental cell lines were purchased from ATCC and cultured in medium based on the product sheet's recommendation. Human cervical cancer cell line HeLa cells (ATCC) were cultured in Eagle's Minimum Essential Medium (EMEM) supplement of 10% heat-inactivated fetal bovine serum (FBS, Gibco) and 1% penicillin streptomycin (P/S, Gibco); IsoMSLN-overexpressing HeLa cells (HeLa-IsoMSLN) were generated by transduction of HeLa cells with Lentivirus expressing IsoMSLN and eGFP and maintained in EMEM medium with 10% FBS and 1% P/S. Human mesothelioma cell line NCI H226 were cultured in RPMI-1640 Medium (Gibo) with 10% FBS and 1% P/S.

Cell transfection and flow cytometry. Lenti-X 293T cells (Takara Bio) were transiently transfected with the Lenti-X plasmid encoding eGFP and MSLN isoform 1 or isoform 2 with Genejuice transfection reagent (EMD Millipore). 3 days after transfection, the transfected cells were harvested, counted, and wash once with FACS buffer, and resuspended in 100 μl FACS buffer containing 0.1 g human FcR blocker (BD Bioscience) per 0.1 million cells for 10 min, 4° C. For anti-isoMSLN mAb binding, the cells were incubated with 100 μl FACS containing anti-iso MSLN mIgG1 mAbs or control mIgG1 (range from 20 μg/ml to 4.096E-07 ug/ml; 1 to 5 dilution), 4° C. for 45 min. Centrifuge cells at 400 g×5 min, carefully remove supernatant; wash cells with 200 μl FACS buffer. Cells were incubated with 100 μl FACS buffer containing APC-conjugated goat anti-mouse 2° antibody (1 to 100 dilution), for 30 min, 4° C. Centrifuge cells at 400 g×5 min, carefully remove supernatant; wash cells with 200 μl FACS buffer for twice. Resuspend the cells pellet in 100 μl FACS buffer. Samples were acquired with NovoCyte 3000 and analyzed by NovoExpress (ACEA Biosciences Inc).

Chimeric antigen receptor (CAR) retrovirus construct and virus packaging. Anti-IsoMSLN CARs were designed as shown in FIG. 3A. The synthesized DNA fragments encoding scFv of anti-IsoMSLN mAb clones #1B6 and 11C11 were inserted into the SFG vector under LTR promoter with restriction enzymes NcoI and BsiWI. All the sequence was verified by DNA sequencing. To generate retrovirus particles, Lenti-X 293T cells were maintained in DMEM+10% FBS, transfected with Genejuice transfection reagent (EMD Millipore) mixed with pEQ-Pam3 package plasmid, RD114 package plasmid and SFG plus CAR plasmid. Virus supernatant were harvested at 48 h and 72 h post transfection, filtered with 45 uM filter and ready for transfection or freeze down in −80° C. for later use.

Generation and expansion of human γδ T cells. Frozen PBMCs from healthy donors (ZenBio) were cultured in CTS™ OpTmizer™ T Cell Expansion with 2% heat-inactivated human serum and 1% P/S and 300 IU/mL human IL-2 (Miltenyi Biotec) and 5 μM zoledronic acid (Sigma). Every 2 to 3 days, cells were feed with fresh culture medium containing human IL-2 300 IU/ml. On day 7, cells were harvested and resuspended in medium and count cells. γδ T cells are further purified using the CliniMACS TCRα/β Kit (Miltenyi Biotec #200-070-410) following the manufacture's protocol. Enriched γδ T cells were subjected to retrovirus transduction or continue expansion with human IL-2 300 IU/ml for additional 7 days.

Retrovirus transduction of γδ T cells. On the day before the transduction, retronectin (Takara Bio) was coated into 6-well non-TC treated plate at 10 μg/ml, 4° C. incubation overnight; 2nd day, remove the retronectin solution, rinse the plate with 2 ml sterile phosphate-buffered saline (PBS) and aspirate PBS; 5 ml fresh or frozen CAR retrovirus supernatant were added into in the retronectin-coated plate, spin 2000× g, 2 h, 32° C.; after removing the virus supernatant and wash plate with fresh medium, 2 million gd-T cells purified on day 7 culture resuspended in CTS medium+5% human serum+P/S plus 300 IU/ml hIL-2 were added into each well; spin 1000× g, 10 min, 32° C.; culture the cells in incubator for additional 7-14 days. The transduced cells were feed with fresh medium with hIL-2 every 2-3 days. The transduction efficiency was determined on day 3-7 post transduction based on the detection of CD34 tag in the CAR construct.

CAR γδ T cell killing assay. IsoMSLN-expressing tumor cells, NCI-H226 were resuspended in γδ T cell culture medium and added into a 96-well flat-bottom plate, then cultured in the incubator overnight. On the following day, CAR γδ T cells or non-transduced γδ T cells were harvested and resuspended the γδ T cell culture medium as above at different cell concentrations. Different Effector to Target (E:T) ratio, and incubated for 48 hours. The CAR-specific γδ T cell cytotoxicity is measured by the LDH release method, using the CyQUANT™ LDH Cytotoxicity Assay (ThermoFisher).

### Example 13: Mesothelin Isoform 2 is a Specific Target for CAR Gamma Delta T Cell Solid Tumor Therapy

### Bioinformatics Analysis Identified Mesothelin Isoform 2 is Cancer Specific Isoform With Unique Protein Peptides.

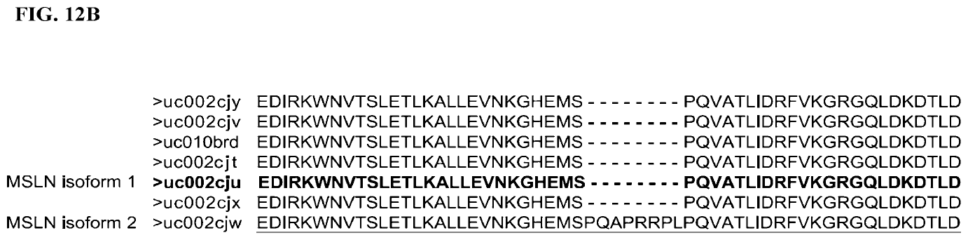

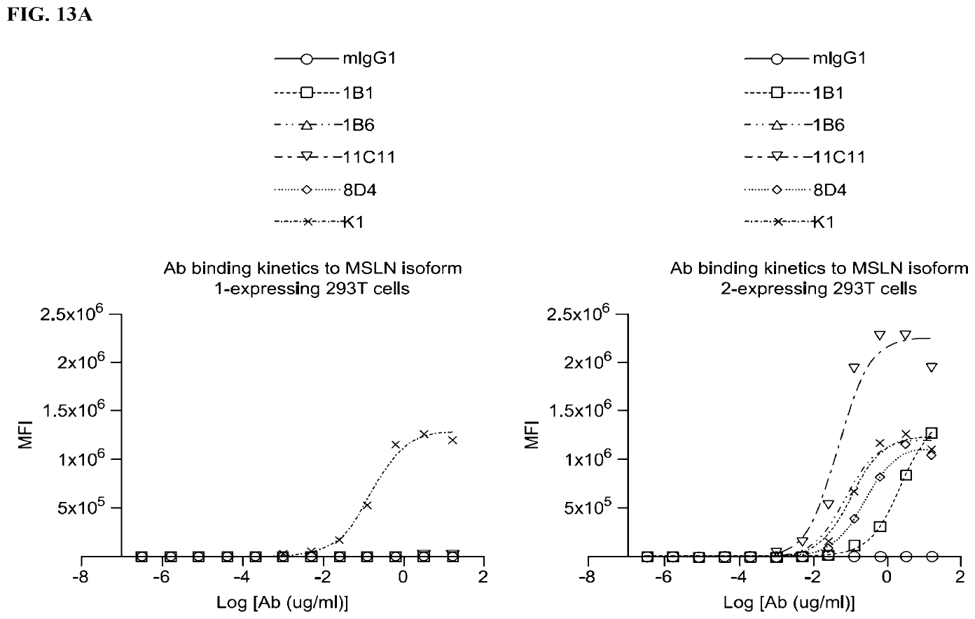

The multi-sequence alignment (MSA) tool of CancerSplice was used to identify all gene isoforms and the unique protein peptides of the cancer specific isoform. MSLN isoform 2 was identified as a tumor-specific isoform with 8 unique amino acids (PQAPRRPL; SEQ ID NO:131) that are absent in the MSLN isoform 1 (transcript variant uc002cjw/ENST00000382862) (FIGS. 12A-12B). To determine if the MSLN isoform 2 unique peptide is present in primary human ovarian tumors, we conducted data mining from public proteomic repositories to confirm using an independent cohort of samples. Of the ovarian tumor samples analyzed, 100% were ovarian serous adenocarcinomas, with 81% grade G3 and 64% stage IIIC and 15% stage IV. The anatomic site of the specimens were 53% ovary, 41% omentum, and 6% peritoneum. The global proteomic data of these specimens were mined for MSLN isoforms. In accordance with the bioinformatic prediction, fragments of the MSLN isoform 2 unique signature peptide RPLPQVATLIDR (SEQ ID NO:132) were detected in 71% of the OV clinical specimens.

### Detection of MSLN Isoform 2 in Cell Lines and Tumor Samples

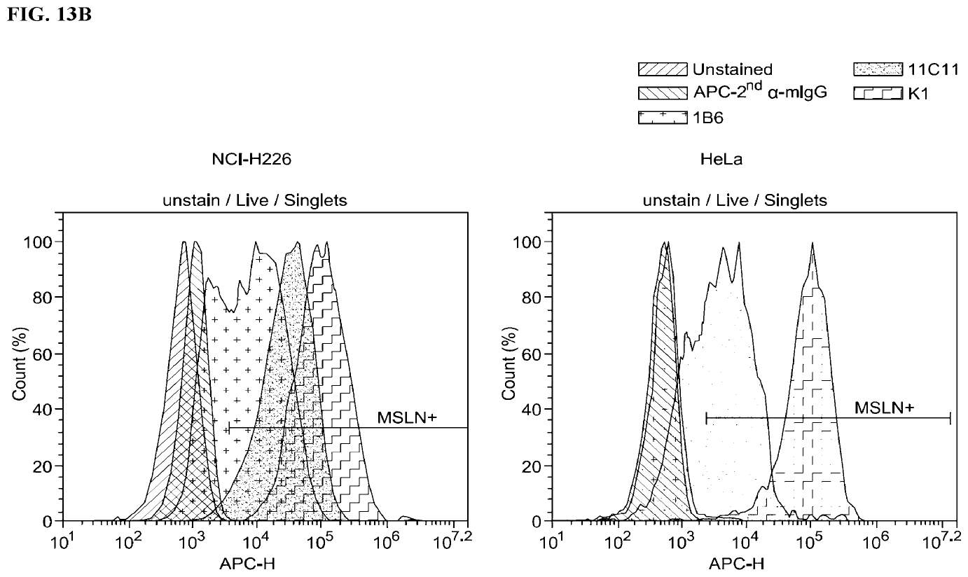

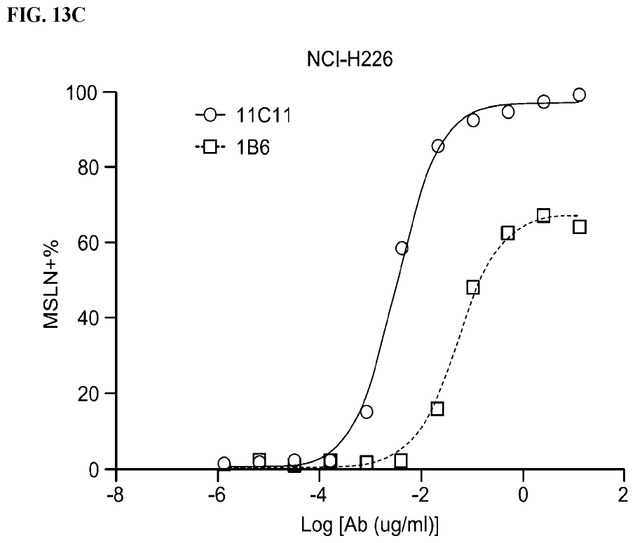

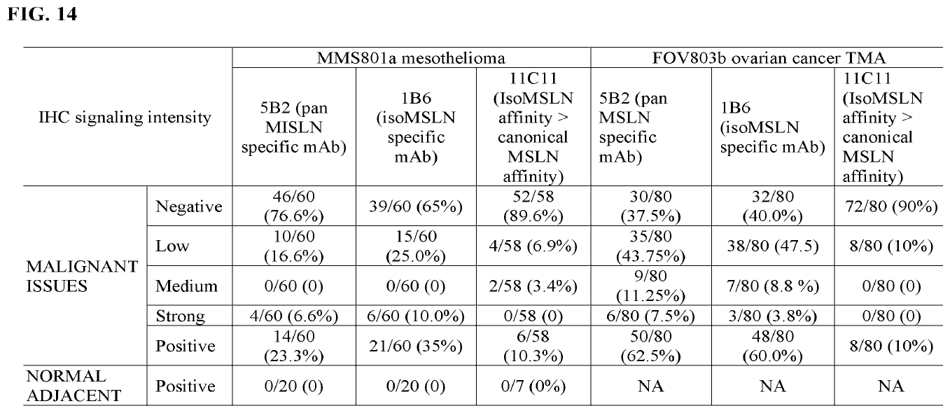

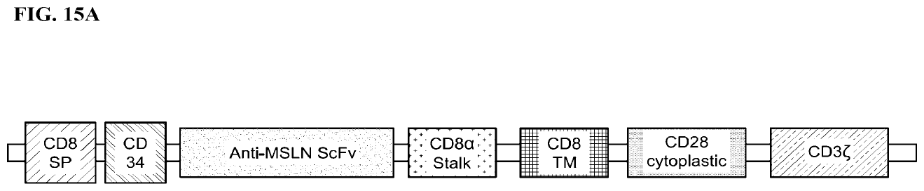

To study the binding pattern of our anti-IsoMSLN monoclonal antibodies (clones 1B6 and 11C11) to tumor cell lines and human primary tumor tissues, we validated the antibody staining procedure and conditions using tumor cell lines with or without MSLN isoform expression. Anti-MSLN antibody 5B2, used in clinical diagnosis of MSLN expression by IHC, was included as a positive control. Both 11C11 and 5B2 detected MSLN isoform 2 expression on IsoMSLN2 expressing tumor cells including 293-T cells overexpressing IsoMSLN2 and NCI H226 with endogenous IsoMSLN 5B2. 11C11, also detected weak expression of MSLN isoform 1 in Hela cells, which lack MSLN isoform 2 expression. 11C11 has weak reactivity to MSLN isoform 1 by flow cytometry and does not detect the MSLN isoform 1 in IHC. In primary ovarian tissues, 11C11 detected strong IsoMSLN expression in ovarian stage IIIc and in tissues from ovarian stage 1 patients but not in normal ovarian tissues. Based on the higher sensitivity shown by clone 11C11 compared with clone 1B6 for cell binding by flow cytometry (FIGS. 13A-13C) and IHC binding patterns, we selected the 11C11 clone for CAR development, and further analyzed its binding specificity to a panel of cancer and normal tissues by IHC using tissue microarrays (TMA). Percentages of 52B, 1B6, and 11C11 DAB staining positive cells were evaluated in two multiple tissue microarrays (TMA) by US Biolab: mesothelioma (MMS801a) and ovarian adenocarcinoma (FOV803b). As a control for normal tissues, TMA cores containing normal adjacent tissues from MMS801a were used. The normal tissues expressing the highest IHC signaling intensity were used as the reference threshold (FIG. 14).

TABLE 2
EC50 of anti-MSLN mAb clones to different MSLN isoforms.

| | |
|---|---|
| Anti-MSLN | |
| mAb | EC50 to bind MSLN (μg/ml) |

| clone # | Isoform 1 | Isoform 2 |
|---|---|---|
| K1 | 0.152 | 0.106 |
| 1B1 | No binding | 2.308 |
| 1B6 | No binding | 0.073 |
| 11C11 | 6.879* | 0.046 |
| 8D4 | No binding | 0.238 |

Percentages of 52B, 1B6, and 11C11 DAB staining positive cells have been evaluated in mesothelioma (MMS801a) and ovarian adenocarcinoma (FOV803b) Tissue Micro Arrays (TMA). Each TMA was processed at full resolution (264.58 mm/px), with a threshold of 0.1 (THC signaling intensity above this threshold was considered positive). All the images were acquired on channel DAB, prefilter Gaussian, with smoothing sigma=2. Percentages were calculated based on the total number of cells for each TMA core. As control on normal tissues, TMA cores containing normal adjacent tissues from MMS801a were used. The normal adjacent expressing the highest IHC signaling intensity was used as the reference threshold). The IHC signaling intensity was used for classifying negative, low, medium and strong positivity.

### CAR γδ T Cells Recognizing MSLN Isoform 2 Show Target-Dependent Killing of Tumor Cells.

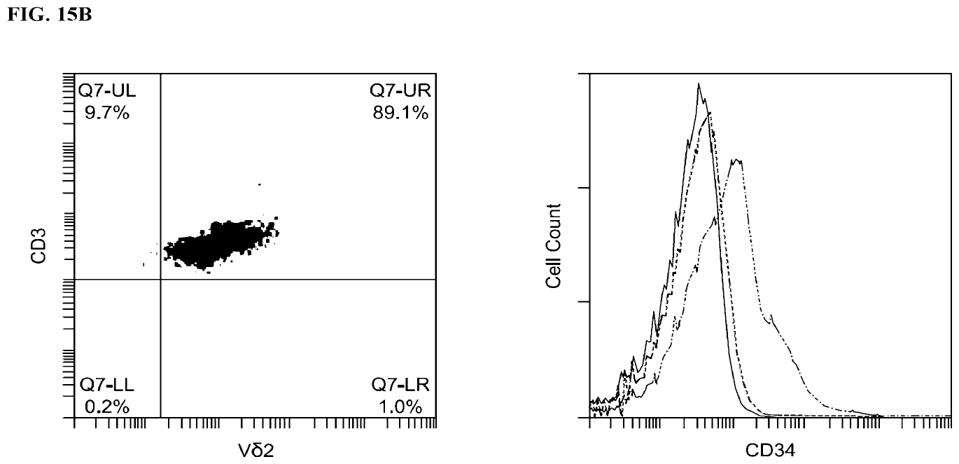

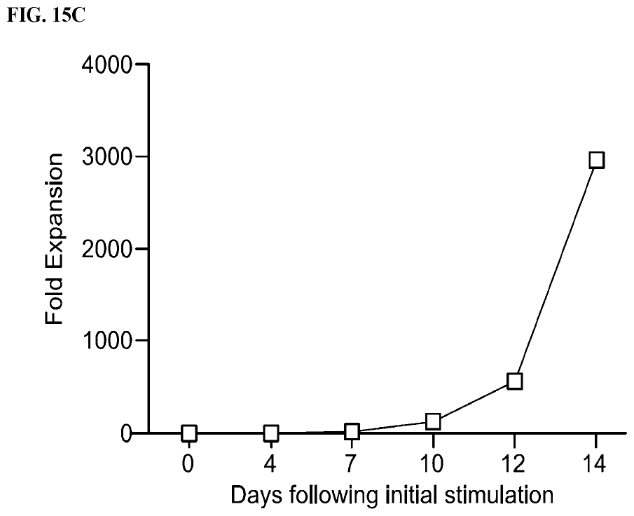

Gamma Delta T (γδ T) cells were analyzed by flow-cytometry to determine purity and transduction efficiency at 48 hours after exposure to the retroviral vector, with the structure shown in FIG. 15A. FIG. 15B shows that the purity of γδ T cells was over 89% with 69% of the cells positive for the CD34 tag (CD34 minimal epitope tagging the N-terminus of the chimeric antigen receptor molecule). During the 14 days of culture in vitro, the γδ T cells expanded more than 2,500-fold (FIG. 15C).

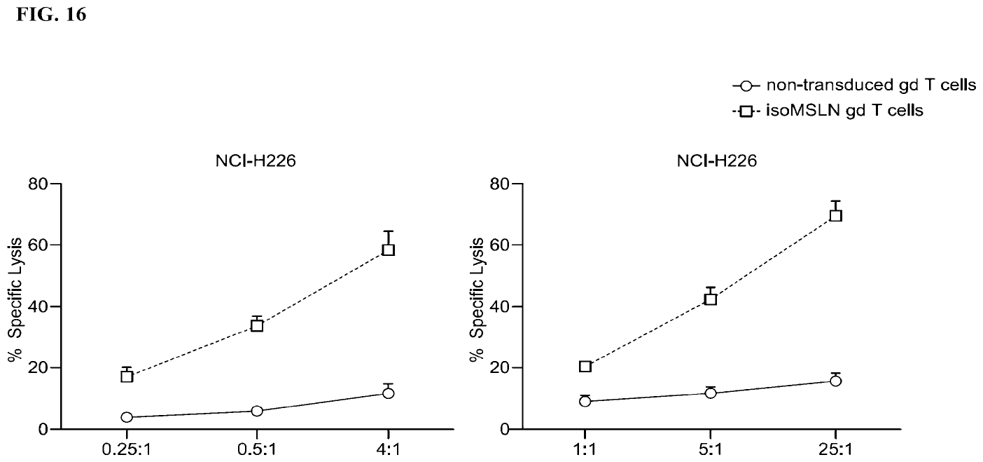

Cytotoxicity of γδ T cells was evaluated to determine potency. FIG. 16 shows the cytotoxicity of the γδ T cells expressing the IsoMSLN receptor with NCI-H226 cells as the target. Anti-IsoMSLN CAR γδ T cells specifically lysed the NCI-H226 cells at low E:T ratios (0.25:1) whereas non-transduced γδ T cells from the same donor did not exhibit significant tumor cell lysis.

### Discussion

It was demonstrated that CAR γδ T cells targeting MSLN isoform 2 can control MSLN isoform 2-expressing tumors without on-target/off-tumor, and without off-target toxicities in vitro and in vivo.

### P EMBODIMENTS

P embodiment 1. A method for generating a T-cell culture enriched for gamma delta T-cells (gdT-cells) comprising: (a) contacting a population of immune cells with a first media composition thereby forming an initial cell culture, wherein said population of immune cells comprises alpha beta T-cells (abT-cells), gdT-cells, and monocytes, wherein the ratio of abT-cells to gdT-cells in said population of immune cells is at least about 16:1, wherein the ratio of monocytes to gdT-cells is at least about 2:1, and wherein said first media composition comprises a bisphosphonate, interleukin-7 (IL-7) and interleukin-2 (IL-2); (b) incubating said initial cell culture for a first time period of about one to about three days, thereby forming a monocyte-depleted immune cell culture; (c) after said first time period, contacting said monocyte-depleted cell culture with interleukin-7 (IL-7) and interleukin-2 (IL-2), but not a bisphosphonate, thereby forming a monocyte-depleted cell culture in contact with a second media composition; (d) incubating said monocyte-depleted cell culture in contact with said second media composition for a second time period of about one to about three days; thereby forming an expanded gdT cell culture, wherein the ratio of abT-cells to gdT-cells in said expanded gdT cell culture is less than about 10:1; (e) removing abT-cells from said expanded gdT cell culture thereby forming an abT-cell-depleted culture; (f) replacing said second media composition with a third media composition comprising interleukin-7 (IL-7) and interleukin-2 (IL-2), wherein said third media composition does not comprise a bisphosphonate; and, (g) incubating said abT-cell-depleted culture for at least one day in contact with said third media composition, thereby forming a T-cell culture enriched for gdT-cells, wherein the ratio of abT-cells to gdT-cells in said T-cell culture enriched for gdT-cells is less than about 1:2.
P embodiment 2. The method of P embodiment 1, wherein said population of immune cells is obtained from peripheral blood.
P embodiment 3. The method of P embodiment 1, wherein said population of immune cells is obtained by fresh leukapheresis.
P embodiment 4. The method of P embodiment 1, wherein said bisphosphonate is zoledronate (zoledronic acid), clodronate, etidronate, alendronate, pamidronate, or neridronate.
P embodiment 5. The method of P embodiment 1, wherein abT-cells are not removed prior to step (e).
P embodiment 6. The method of P embodiment 1, wherein the concentration of IL-7 is about 250 U/mL.
P embodiment 7. The method of P embodiment 1, wherein the ratio of abT-cells to gdT-cells of said expanded gdT cell culture is less than about 5:1.
P embodiment 8. The method of P embodiment 1, wherein the ratio of abT-cells to gdT-cells of said expanded gdT cell culture is less than about 1:1
P embodiment 9. The method of P embodiment 1, wherein the ratio of abT-cells to gdT-cells of said T-cell culture enriched for gdT-cells is less than about 1:5.
P embodiment 10. The method of P embodiment 1, wherein the ratio of abT-cells to gdT-cells of said T-cell culture enriched for gdT-cells is less than about 1:10.
P embodiment 11. The method of P embodiment 1, wherein the ratio of abT-cells to gdT-cells of said T-cell culture enriched for gdT-cells is less than about 1:50.
P embodiment 12. The method of P embodiment 1, wherein the ratio of abT-cells to gdT-cells of said T-cell culture enriched for gdT-cells is less than about 1:100.
P embodiment 13. The method of P embodiment 1, wherein the ratio of abT-cells to gdT-cells of said T-cell culture enriched for gdT-cells is less than about 1:500.
P embodiment 14. The method of P embodiment 1, wherein the ratio of abT-cells to gdT-cells of said T-cell culture enriched for gdT-cells is less than about 1:1000.
P embodiment 15. The method of P embodiment 1, wherein the percentage of abT-cells of total cells in said T-cell culture enriched for gdT-cells is about 1% or less.
P embodiment 16. The method of any of P embodiments 1 to 15, said method further comprising incubating said abT-cell-depleted culture 2, 3, 4, 5, or 6 days.
P embodiment 17. A method of generating a gdT-cell expressing a Chimeric Antigen Receptor (CAR), said method comprising introducing a nucleic acid encoding a CAR to a gdT-cell obtained by any of the methods of embodiments 1 to 16.
P embodiment 18. A T-cell culture enriched for gdT cells obtained by any of the methods of P embodiments 1 to 16.
P embodiment 19. A T-cell population expressing a CAR, comprising the T-cell culture enriched for gdT cells of P embodiment 18, wherein the gdT cells comprise a nucleic acid encoding said CAR.
P embodiment 20. The T-cell population of P embodiment 19, wherein said CAR comprises an anti-IsoMSLN binding molecule.
P embodiment 21. The T-cell population of P embodiment 19, wherein said CAR comprises a chimeric PD1 binding molecule.

### EMBODIMENTS

Embodiment 1. A method for generating a T-cell culture enriched for gamma delta T-cells (gdT-cells) comprising: (a) contacting a population of immune cells with a first media composition thereby forming an initial cell culture, wherein said population of immune cells comprises alpha beta T-cells (abT-cells), gdT-cells, and monocytes, wherein the ratio of abT-cells to gdT-cells in said population of immune cells is at least about 16:1, wherein the ratio of monocytes to gdT-cells is at least about 2:1, and wherein said first media composition comprises a bisphosphonate, interleukin-7 (IL-7) and interleukin-2 (IL-2); (b) incubating said initial cell culture for a first time period of about one to about three days, thereby forming a monocyte-depleted immune cell culture; (c) after said first time period, contacting said monocyte-depleted cell culture with interleukin-7 (IL-7) and interleukin-2 (IL-2), but not a bisphosphonate, thereby forming a monocyte-depleted cell culture in contact with a second media composition; (d) incubating said monocyte-depleted cell culture in contact with said second media composition for a second time period of about one to about three days; thereby forming an expanded gdT cell culture, wherein the ratio of abT-cells to gdT-cells in said expanded gdT cell culture is less than about 10:1; (e) removing abT-cells from said expanded gdT cell culture thereby forming an abT-cell-depleted culture; (f) replacing said second media composition with a third media composition comprising interleukin-7 (IL-7) and interleukin-2 (IL-2), wherein said third media composition does not comprise a bisphosphonate; and, (g) incubating said abT-cell-depleted culture for at least one day in contact with said third media composition, thereby forming a T-cell culture enriched for gdT-cells, wherein the ratio of abT-cells to gdT-cells in said T-cell culture enriched for gdT-cells is less than about 1:2.
Embodiment 2. The method of embodiment 1, wherein said population of immune cells is obtained from peripheral blood.
Embodiment 3. The method of embodiment 1, wherein said population of immune cells is obtained by fresh leukapheresis.
Embodiment 4. The method of embodiment 1, wherein said bisphosphonate is zoledronate (zoledronic acid), clodronate, etidronate, alendronate, pamidronate, or neridronate.
Embodiment 5. The method of embodiment 1, wherein abT-cells are not removed prior to step (e).
Embodiment 6. The method of embodiment 1, wherein the concentration of IL-7 is about 250 U/mL.
Embodiment 7. The method of embodiment 1, wherein the ratio of abT-cells to gdT-cells of said expanded gdT cell culture is less than about 5:1.
Embodiment 8. The method of embodiment 1, wherein the ratio of abT-cells to gdT-cells of said expanded gdT cell culture is less than about 1:1
Embodiment 9. The method of embodiment 1, wherein the ratio of abT-cells to gdT-cells of said T-cell culture enriched for gdT-cells is less than about 1:5.
Embodiment 10. The method of embodiment 1, wherein the ratio of abT-cells to gdT-cells of said T-cell culture enriched for gdT-cells is less than about 1:10.
Embodiment 11. The method of embodiment 1, wherein the ratio of abT-cells to gdT-cells of said T-cell culture enriched for gdT-cells is less than about 1:50.
Embodiment 12. The method of embodiment 1, wherein the ratio of abT-cells to gdT-cells of said T-cell culture enriched for gdT-cells is less than about 1:100.
Embodiment 13. The method of embodiment 1, wherein the ratio of abT-cells to gdT-cells of said T-cell culture enriched for gdT-cells is less than about 1:500.
Embodiment 14. The method of embodiment 1, wherein the ratio of abT-cells to gdT-cells of said T-cell culture enriched for gdT-cells is less than about 1:1000.
Embodiment 15. The method of embodiment 1, wherein the percentage of abT-cells of total cells in said T-cell culture enriched for gdT-cells is about 1% or less.
Embodiment 16. The method of any of embodiments 1 to 15, said method further comprising incubating said abT-cell-depleted culture 2, 3, 4, 5, or 6 days.
Embodiment 17. The method of any of embodiments 1 to 16, said method further comprising: (h) harvesting and cryopreserving said T-cell culture enriched for gdT-cells.
Embodiment 18. The method of embodiment 17, wherein said cryopreserving comprises suspending said T-cell culture enriched for gdT-cells in a cryopreservation medium.
Embodiment 19. The method of embodiment 17 or 18, wherein said cryopreservation medium comprises between about 5% and about 10% dimethylsulfoxide (DMSO).
Embodiment 20. A method of generating a gdT-cell expressing a Chimeric Antigen Receptor (CAR), said method comprising introducing a nucleic acid encoding a CAR to a gdT-cell obtained by any of the methods of embodiments 1 to 19.
Embodiment 21. A T-cell culture enriched for gdT cells obtained by any of the methods of embodiments 1 to 19.
Embodiment 22. A T-cell population expressing a Chimeric Antigen Receptor (CAR), comprising the T-cell culture enriched for gdT cells of embodiment 21, wherein the gdT cells comprise a nucleic acid encoding said CAR.
Embodiment 23. The T-cell population of embodiment 22, wherein said CAR comprises an anti-IsoMSLN binding molecule.
Embodiment 24. The T-cell population of embodiment 22, wherein said CAR comprises a chimeric PD1 binding molecule.

What is claimed is:

1. A method for generating a T-cell culture enriched for gamma delta T-cells (gdT-cells) comprising:
(a) contacting a population of immune cells with a first media composition thereby forming an initial cell culture, wherein said population of immune cells comprises alpha beta T-cells (abT-cells), gdT-cells, and monocytes, wherein the ratio of abT-cells to gdT-cells in said population of immune cells is at least about 16:1, wherein the ratio of monocytes to gdT-cells is at least about 2:1, and wherein said first media composition comprises a bisphosphonate, interleukin-7 (IL-7) and interleukin-2 (IL-2);
(b) incubating said initial cell culture for a first time period of about one to about three days, thereby forming a monocyte-depleted immune cell culture;
(c) after said first time period, contacting said monocyte-depleted cell culture with interleukin-7 (IL-7) and interleukin-2 (IL-2), but not a bisphosphonate, thereby forming a monocyte-depleted cell culture in contact with a second media composition;
(d) incubating said monocyte-depleted cell culture in contact with said second media composition for a second time period of about one to about three days;
thereby forming an expanded gdT cell culture, wherein the ratio of abT-cells to gdT-cells in said expanded gdT cell culture is less than about 10:1;
(e) removing abT-cells from said expanded gdT cell culture thereby forming an abT-cell-depleted culture;
(f) replacing said second media composition with a third media composition comprising interleukin-7 (IL-7) and interleukin-2 (IL-2), wherein said third media composition does not comprise a bisphosphonate; and,
(g) incubating said abT-cell-depleted culture for at least one day in contact with said third media composition, thereby forming a T-cell culture enriched for gdT-cells, wherein the ratio of abT-cells to gdT-cells in said T-cell culture enriched for gdT-cells is less than about 1:2.

2. The method of claim 1, wherein said population of immune cells is obtained from peripheral blood.

3. The method of claim 1, wherein said population of immune cells is obtained by fresh leukapheresis.

4. The method of claim 1, wherein said bisphosphonate is zoledronate (zoledronic acid), clodronate, etidronate, alendronate, pamidronate, or neridronate.

5. The method of claim 1, wherein abT-cells are not removed prior to step (e).

6. The method of claim 1, wherein the concentration of IL-7 is about 250 U/mL.

7. The method of claim 1, wherein the ratio of abT-cells to gdT-cells of said expanded gdT cell culture is less than about 5:1.

8. The method of claim 1, wherein the ratio of abT-cells to gdT-cells of said expanded gdT cell culture is less than about 1:1.

9. The method of claim 1, wherein the ratio of abT-cells to gdT-cells of said T-cell culture enriched for gdT-cells is less than about 1:5.

10. The method of claim 1, wherein the ratio of abT-cells to gdT-cells of said T-cell culture enriched for gdT-cells is less than about 1:10.

11. The method of claim 1, wherein the ratio of abT-cells to gdT-cells of said T-cell culture enriched for gdT-cells is less than about 1:50.

12. The method of claim 1, wherein the ratio of abT-cells to gdT-cells of said T-cell culture enriched for gdT-cells is less than about 1:100.

13. The method of claim 1, wherein the ratio of abT-cells to gdT-cells of said T-cell culture enriched for gdT-cells is less than about 1:500.

14. The method of claim 1, wherein the ratio of abT-cells to gdT-cells of said T-cell culture enriched for gdT-cells is less than about 1:1000.

15. The method of claim 1, wherein the percentage of abT-cells of total cells in said T-cell culture enriched for gdT-cells is about 1% or less.

16. The method of any of claims 1 to 15, said method further comprising incubating said abT-cell-depleted culture 2, 3, 4, 5, or 6 days.

17. The method of any of claims 1 to 16, said method further comprising: (h) cryopreserving said T-cell culture enriched for gdT-cells.

18. The method of claim 17, wherein said cryopreserving comprises suspending said T-cell culture enriched for gdT-cells in a cryopreservation medium.

19. The method of claim 17 or 18, wherein said cryopreservation medium comprises between about 5% and about 10% dimethylsulfoxide (DMSO).

20. A method of generating a gdT-cell expressing a Chimeric Antigen Receptor (CAR), said method comprising introducing a nucleic acid encoding a CAR to a gdT-cell obtained by any of the methods of claims 1 to 19.

21. A T-cell culture enriched for gdT cells obtained by any of the methods of claims 1 to 19.

22. A T-cell population expressing a Chimeric Antigen Receptor (CAR), comprising the T-cell culture enriched for gdT cells of claim 21, wherein the gdT cells comprise a nucleic acid encoding said CAR.

23. The T-cell population of claim 22, wherein said CAR comprises an anti-IsoMSLN binding molecule.

24. The T-cell population of claim 22, wherein said CAR comprises a chimeric PD1 binding molecule.


SEQUENCE LISTING (220 sequences)

SEQ ID NO: 1 - protein, 134 aa, Synthetic construct
MNFGFSLIFLVLVLKGVQCEVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSA

SEQ ID NO: 2 - protein, 115 aa, Synthetic construct
EVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSA

SEQ ID NO: 3 - protein, 5 aa, Synthetic construct
RYAMS

SEQ ID NO: 4 - protein, 16 aa, Synthetic construct
SITFGGDPYYPDGLKG

SEQ ID NO: 5 - protein, 7 aa, Synthetic construct
HESWFAY

SEQ ID NO: 6 - protein, 30 aa, Synthetic construct
EVKLVESGGGLVKPGGSLKLSCEASGFTFS

SEQ ID NO: 7 - protein, 14 aa, Synthetic construct
WVRHTPEKRLAWVA

SEQ ID NO: 8 - protein, 32 aa, Synthetic construct
RFTISRDNTRNILYLQMNSLRSEDTAMYYCVR

SEQ ID NO: 9 - protein, 11 aa, Synthetic construct
WGQGTLVTVSA

SEQ ID NO: 10 - protein, 131 aa, Synthetic construct
MKLPVRLLVLMFWIPASSSDVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIK

SEQ ID NO: 11 - protein, 112 aa, Synthetic construct
DVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIK

SEQ ID NO: 12 - protein, 16 aa, Synthetic construct
RSSQTIVHTDGNIYLE

SEQ ID NO: 13 - protein, 7 aa, Synthetic construct
KISNRFS

SEQ ID NO: 14 - protein, 9 aa, Synthetic construct
FQASHVPYT

SEQ ID NO: 15 - protein, 23 aa, Synthetic construct
DVLMTQTPLSLPVSLGEQVSISC

SEQ ID NO: 16 - protein, 15 aa, Synthetic construct
WYLQNPGQSPRLLIY

SEQ ID NO: 17 - protein, 32 aa, Synthetic construct
GVPDRFSGSGSGTDFTLKISRVEAEDLGIYYC

SEQ ID NO: 18 - protein, 10 aa, Synthetic construct
FGGGTKLEIK

SEQ ID NO: 19 - DNA, 402 bp, Synthetic construct
  source 1..402: mol_type = other DNA
atgaacttcgggttcagcttgattttccttgtccttgttttaaaaggtgtccagtgtgaagtgaagctggtggagtctgggggaggcttagtgaagcctggggggtccctgaaactctcctgtgaggcctctgggttcactttcagtcgctatgccatgtcttgggttcgccatactccagagaagagactggcctgggtcgcatccataacttttggtggtgacccgtattatcccgacggtttgaagggccgattcaccatctccagagataataccaggaacatcctgtacctgcaaatgaacagtctgaggtctgaagacacggccatgtattattgtgtacgacacgagtcatggtttgcttactggggccaagggactctggtcactgtctctgca

SEQ ID NO: 20 - DNA, 345 bp, Synthetic construct
  source 1..345: mol_type = other DNA
gaagtgaagctggtggagtctgggggaggcttagtgaagcctggggggtccctgaaactctcctgtgaggcctctgggttcactttcagtcgctatgccatgtcttgggttcgccatactccagagaagagactggcctgggtcgcatccataacttttggtggtgacccgtattatcccgacggtttgaagggccgattcaccatctccagagataataccaggaacatcctgtacctgcaaatgaacagtctgaggtctgaagacacggccatgtattattgtgtacgacacgagtcatggtttgcttactggggccaagggactctggtcactgtctctgca

SEQ ID NO: 21 - DNA, 15 bp, Synthetic construct
  source 1..15: mol_type = other DNA
cgctatgccatgtct

SEQ ID NO: 22 - DNA, 48 bp, Synthetic construct
  source 1..48: mol_type = other DNA
tccataacttttggtggtgacccgtattatcccgacggtttgaagggc

SEQ ID NO: 23 - DNA, 21 bp, Synthetic construct
  source 1..21: mol_type = other DNA
cacgagtcatggtttgcttac

SEQ ID NO: 24 - DNA, 90 bp, Synthetic construct
  source 1..90: mol_type = other DNA
gaagtgaagctggtggagtctgggggaggcttagtgaagcctggggggtccctgaaactctcctgtgaggcctctgggttcactttcagt

SEQ ID NO: 25 - DNA, 42 bp, Synthetic construct
  source 1..42: mol_type = other DNA
tgggttcgccatactccagagaagagactggcctgggtcgca

SEQ ID NO: 26 - DNA, 96 bp, Synthetic construct
  source 1..96: mol_type = other DNA
cgattcaccatctccagagataataccaggaacatcctgtacctgcaaatgaacagtctgaggtctgaagacacggccatgtattattgtgtacga

SEQ ID NO: 27 - DNA, 33 bp, Synthetic construct
  source 1..33: mol_type = other DNA
tggggccaagggactctggtcactgtctctgca

SEQ ID NO: 28 - DNA, 393 bp, Synthetic construct
  source 1..393: mol_type = other DNA
atgaagttgcctgttaggctgttggtgctgatgttctggattcctgcttccagcagtgatgttttgatgacccaaactccactctccctgcctgtcagtcttggagaacaagtctccatctcttgcagatctagtcagaccattgtacatactgatggcaacatctatttagaatggtacctccagaacccaggccagtctcctaggctcctgatctacaaaatttccaaccgattttctggggtccctgacaggttcagtggcagtggatcagggacagatttcacactcaagatcagcagagtggaggctgaggatctgggaatttattactgctttcaagcttcacatgttccgtacacgttcggaggggggaccaagctggaaataaaa

SEQ ID NO: 29 - DNA, 336 bp, Synthetic construct
  source 1..336: mol_type = other DNA
gatgttttgatgacccaaactccactctccctgcctgtcagtcttggagaacaagtctccatctcttgcagatctagtcagaccattgtacatactgatggcaacatctatttagaatggtacctccagaacccaggccagtctcctaggctcctgatctacaaaatttccaaccgattttctggggtccctgacaggttcagtggcagtggatcagggacagatttcacactcaagatcagcagagtggaggctgaggatctgggaatttattactgctttcaagcttcacatgttccgtacacgttcggaggggggaccaagctggaaataaaa

SEQ ID NO: 30 - DNA, 48 bp, Synthetic construct
  source 1..48: mol_type = other DNA
agatctagtcagaccattgtacatactgatggcaacatctatttagaa

SEQ ID NO: 31 - DNA, 21 bp, Synthetic construct
  source 1..21: mol_type = other DNA
aaaatttccaaccgattttct

SEQ ID NO: 32 - DNA, 27 bp, Synthetic construct
  source 1..27: mol_type = other DNA
tttcaagcttcacatgttccgtacacg

SEQ ID NO: 33 - DNA, 69 bp, Synthetic construct
  source 1..69: mol_type = other DNA
gatgttttgatgacccaaactccactctccctgcctgtcagtcttggagaacaagtctccatctcttgc

SEQ ID NO: 34 - DNA, 45 bp, Synthetic construct
  source 1..45: mol_type = other DNA
tggtacctccagaacccaggccagtctcctaggctcctgatctac

SEQ ID NO: 35 - DNA, 96 bp, Synthetic construct
  source 1..96: mol_type = other DNA
ggggtccctgacaggttcagtggcagtggatcagggacagatttcacactcaagatcagcagagtggaggctgaggatctgggaatttattactgc

SEQ ID NO: 36 - DNA, 30 bp, Synthetic construct
  source 1..30: mol_type = other DNA
ttcggaggggggaccaagctggaaataaaa

SEQ ID NO: 37 - protein, 134 aa, Synthetic construct
MNFGFSLIFLVLVLKGVQCEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSA

SEQ ID NO: 38 - protein, 115 aa, Synthetic construct
EVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSA

SEQ ID NO: 39 - protein, 5 aa, Synthetic construct
RYAMS

SEQ ID NO: 40 - protein, 16 aa, Synthetic construct
SITFGGSAYYLDSVKG

SEQ ID NO: 41 - protein, 7 aa, Synthetic construct
HQPWFDY

SEQ ID NO: 42 - protein, 30 aa, Synthetic construct
EVKLVESGGGLVKPGGSLKVSCTASGFTFS

SEQ ID NO: 43 - protein, 14 aa, Synthetic construct
WVRQTPERRLEWVA

SEQ ID NO: 44 - protein, 32 aa, Synthetic construct
RFTISRDNAQNILYLQMNSLVSEDTAIYNCAR

SEQ ID NO: 45 - protein, 11 aa, Synthetic construct
WGQGTLVTVSA

SEQ ID NO: 46 - protein, 131 aa, Synthetic construct
  VARIANT 49: note = x is i or v
MNLPIRLLVLMFWIPASSSDVLLTQTPLSLPVSLGDQASISCRSSQNIXHSDGDTYLDWFLQKPGQSPNLLIYKVSNRFSGVPDRFSGSGSGTDFTLKISRVEADDLGVYYCFQASHVPYTFGGGTKLEVK

SEQ ID NO: 47 - protein, 112 aa, Synthetic construct
  VARIANT 30: note = x is i or v
DVLLTQTPLSLPVSLGDQASISCRSSQNIXHSDGDTYLDWFLQKPGQSPNLLIYKVSNRFSGVPDRFSGSGSGTDFTLKISRVEADDLGVYYCFQASHVPYTFGGGTKLEVK

SEQ ID NO: 48 - protein, 16 aa, Synthetic construct
  VARIANT 7: note = x is i or v
RSSQNIXHSDGDTYLD

SEQ ID NO: 49 - protein, 7 aa, Synthetic construct
KVSNRFS

SEQ ID NO: 50 - protein, 9 aa, Synthetic construct
FQASHVPYT

SEQ ID NO: 51 - protein, 23 aa, Synthetic construct
DVLLTQTPLSLPVSLGDQASISC

SEQ ID NO: 52 - protein, 15 aa, Synthetic construct
WFLQKPGQSPNLLIY

SEQ ID NO: 53 - protein, 32 aa, Synthetic construct
GVPDRFSGSGSGTDFTLKISRVEADDLGVYYC

SEQ ID NO: 54 - protein, 10 aa, Synthetic construct
FGGGTKLEVK

SEQ ID NO: 55 - DNA, 402 bp, Synthetic construct
  source 1..402: mol_type = other DNA
atgaacttcgggttcagcttgattttccttgtccttgttttaaaaggtgtccagtgcgaagtgaagctggtggagtctgggggaggcttagtgaagcctggagggtccctgaaagtctcctgtacagcctctggattcactttcagtcgttatgccatgtcttgggttcgccagactccagagaggaggctggagtgggtcgcatccattacttttggtggtagcgcctactatttagacagtgtgaagggccgattcaccatctccagagataatgcccagaacatcctgtacctgcaaatgaacagtctggtgtctgaggacacggccatttataactgtgcaagacaccagccctggtttgattactggggccaagggactctggtcactgtctctgca

SEQ ID NO: 56 - DNA, 345 bp, Synthetic construct
  source 1..345: mol_type = other DNA
gaagtgaagctggtggagtctgggggaggcttagtgaagcctggagggtccctgaaagtctcctgtacagcctctggattcactttcagtcgttatgccatgtcttgggttcgccagactccagagaggaggctggagtgggtcgcatccattacttttggtggtagcgcctactatttagacagtgtgaagggccgattcaccatctccagagataatgcccagaacatcctgtacctgcaaatgaacagtctggtgtctgaggacacggccatttataactgtgcaagacaccagccctggtttgattactggggccaagggactctggtcactgtctctgca

SEQ ID NO: 57 - DNA, 15 bp, Synthetic construct
  source 1..15: mol_type = other DNA
cgttatgccatgtct

SEQ ID NO: 58 - DNA, 48 bp, Synthetic construct
  source 1..48: mol_type = other DNA
tccattacttttggtggtagcgcctactatttagacagtgtgaagggc

SEQ ID NO: 59 - DNA, 21 bp, Synthetic construct
  source 1..21: mol_type = other DNA
caccagccctggtttgattac

SEQ ID NO: 60 - DNA, 90 bp, Synthetic construct
  source 1..90: mol_type = other DNA
gaagtgaagctggtggagtctgggggaggcttagtgaagcctggagggtccctgaaagtctcctgtacagcctctggattcactttcagt

SEQ ID NO: 61 - DNA, 42 bp, Synthetic construct
  source 1..42: mol_type = other DNA
tgggttcgccagactccagagaggaggctggagtgggtcgca

SEQ ID NO: 62 - DNA, 96 bp, Synthetic construct
  source 1..96: mol_type = other DNA
cgattcaccatctccagagataatgcccagaacatcctgtacctgcaaatgaacagtctggtgtctgaggacacggccatttataactgtgcaaga

SEQ ID NO: 63 - DNA, 33 bp, Synthetic construct; identical to SEQ ID NO: 27
  source 1..33: mol_type = other DNA

SEQ ID NO: 64 - DNA, 393 bp, Synthetic construct
  source 1..393: mol_type = other DNA
  misc_difference 145: note = r is a or g
  misc_difference 147: note = w is a or t
atgaacttgcctattaggctgttggtgctgatgttctggattcctgcttccagcagtgatgtcttgctgacccagactccactctccctgcctgtcagtcttggagatcaggcctccatctcttgcagatctagtcagaacattrtwcatagtgatggagacacctatttagactggttcctgcagaaaccaggccagtctccaaacctcctaatctacaaagtttccaaccgattttctggggtcccagacaggttcagtggcagtggatcagggacagatttcaccctcaagatcagcagagtggaggctgacgatctgggagtttattactgctttcaagcttcacatgttccgtacacgttcggaggggggaccaagctggaagtaaaa

SEQ ID NO: 65 - DNA, 336 bp, Synthetic construct
  source 1..336: mol_type = other DNA
  misc_difference 88: note = r is a or g
  misc_difference 90: note = w is a or t
gatgtcttgctgacccagactccactctccctgcctgtcagtcttggagatcaggcctccatctcttgcagatctagtcagaacattrtwcatagtgatggagacacctatttagactggttcctgcagaaaccaggccagtctccaaacctcctaatctacaaagtttccaaccgattttctggggtcccagacaggttcagtggcagtggatcagggacagatttcaccctcaagatcagcagagtggaggctgacgatctgggagtttattactgctttcaagcttcacatgttccgtacacgttcggaggggggaccaagctggaagtaaaa

SEQ ID NO: 66 - DNA, 48 bp, Synthetic construct
  source 1..48: mol_type = other DNA
  misc_difference 21: note = w is a or t
  misc_difference 19: note = r is a or g
agatctagtcagaacattrtwcatagtgatggagacacctatttagac

SEQ ID NO: 67 - DNA, 21 bp, Synthetic construct
  source 1..21: mol_type = other DNA
aaagtttccaaccgattttct

SEQ ID NO: 68 - DNA, 27 bp, Synthetic construct
  source 1..27: mol_type = other DNA
tttcaagcttcacatgttccgtacacg

SEQ ID NO: 69 - DNA, 69 bp, Synthetic construct
  source 1..69: mol_type = other DNA
gatgtcttgctgacccagactccactctccctgcctgtcagtcttggagatcaggcctccatctcttgc

SEQ ID NO: 70 - DNA, 45 bp, Synthetic construct
  source 1..45: mol_type = other DNA
tggttcctgcagaaaccaggccagtctccaaacctcctaatctac

SEQ ID NO: 71 - DNA, 96 bp, Synthetic construct
  source 1..96: mol_type = other DNA
ggggtcccagacaggttcagtggcagtggatcagggacagatttcaccctcaagatcagcagagtggaggctgacgatctgggagtttattactgc

SEQ ID NO: 72 - DNA, 30 bp, Synthetic construct
  source 1..30: mol_type = other DNA
ttcggaggggggaccaagctggaagtaaaa

SEQ ID NO: 73 - protein, 521 aa, Synthetic construct
MALPVTALLLPLALLLHAARPGSELPTQGTFSNVSTNVSSPEVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSAGGGGSGGGGSGGGGSDVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1563 bp, Synthetic construct
  source 1..1563: mol_type = other DNA
atggctcttccggtaaccgctctgctgcttcctttggctctgcttcttcacgcggctaggccagggagcgaattgcctacgcaggggactttttcaaatgtaagcacgaatgtgagctctccggaagtgaaattggtagagtctggtgggggactggttaaacctggaggcagtctgaagctctcctgcgaagccagcggctttacgttcagtcggtacgctatgtcctgggtgcgacacacaccagaaaagcgactcgcttgggtagcgagcataacgtttgggggcgatccttattacccggatggtttgaaaggtcgcttcactatcagtcgagataatacgcgaaacattctttatcttcaaatgaatagcctgcgctctgaagacaccgcgatgtattattgtgttaggcatgagtcttggtttgcatactggggtcagggtactcttgtcactgtatctgccggaggtgggggtagcggcggcggaggtagtgggggaggcggcagcgacgtgttgatgactcaaactccgcttagcttgccagtgtctcttggagaacaggtctccatatcatgtcgatccagtcaaactatcgttcataccgatgggaatatctatctcgaatggtatctgcaaaatccagggcagagtccccggcttctgatatataagattagcaatagatttagtggtgttcccgacaggttttcaggttctgggtccggaacggattttaccctgaagataagtcgagttgaagcggaagacctgggcatatactattgcttccaggcgtcccacgtcccctatacatttgggggtgggactaaactggaaattaaacgtacgacgacaccagccccaagacccccaactcccgctcctaccatagcttcccaacccctgtcactgaggccagaggcatgcaggcccgctgcgggtggcgcggtacatacgcggggactggactttgcatgtgacatttacatatgggcgccactggcgggaacatgtggagttttgttgcttagcctggtcataacactgtattgcaatcatcgcaacagacgccgcgtctgcaagtgtcccagggtcgacagatctaagagaagtagacttcttcacagtgattatatgaatatgacgcctcgaagacccggcccgacacgcaaacactatcagccgtatgccccccctcgggattttgctgcctaccgcagccgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctagg

SEQ ID NO: 75 - protein, 21 aa, Synthetic construct
MALPVTALLLPLALLLHAARP

SEQ ID NO: 76 - DNA, 63 bp, Synthetic construct
  source 1..63: mol_type = other DNA
atggctcttccggtaaccgctctgctgcttcctttggctctgcttcttcacgcggctaggcca

SEQ ID NO: 77-78 - skipped (2 entries)

SEQ ID NO: 79 - protein, 16 aa, Synthetic construct
ELPTQGTFSNVSTNVS

SEQ ID NO: 80 - DNA, 48 bp, Synthetic construct
  source 1..48: mol_type = other DNA
gaattgcctacgcaggggactttttcaaatgtaagcacgaatgtgagc

SEQ ID NO: 81-82 - skipped (2 entries)

SEQ ID NO: 83 - protein, 115 aa, Synthetic construct; identical to SEQ ID NO: 2

SEQ ID NO: 84 - DNA, 345 bp, Synthetic construct
  source 1..345: mol_type = other DNA
gaagtgaaattggtagagtctggtgggggactggttaaacctggaggcagtctgaagctctcctgcgaagccagcggctttacgttcagtcggtacgctatgtcctgggtgcgacacacaccagaaaagcgactcgcttgggtagcgagcataacgtttgggggcgatccttattacccggatggtttgaaaggtcgcttcactatcagtcgagataatacgcgaaacattctttatcttcaaatgaatagcctgcgctctgaagacaccgcgatgtattattgtgttaggcatgagtcttggtttgcatactggggtcagggtactcttgtcactgtatctgcc

SEQ ID NO: 85 - protein, 15 aa, Synthetic construct
GGGGSGGGGSGGGGS

SEQ ID NO: 86 - DNA, 45 bp, Synthetic construct
  source 1..45: mol_type = other DNA
ggaggtgggggtagcggcggcggaggtagtgggggaggcggcagc

SEQ ID NO: 87 - protein, 112 aa, Synthetic construct; identical to SEQ ID NO: 11

SEQ ID NO: 88 - DNA, 336 bp, Synthetic construct
  source 1..336: mol_type = other DNA
gacgtgttgatgactcaaactccgcttagcttgccagtgtctcttggagaacaggtctccatatcatgtcgatccagtcaaactatcgttcataccgatgggaatatctatctcgaatggtatctgcaaaatccagggcagagtccccggcttctgatatataagattagcaatagatttagtggtgttcccgacaggttttcaggttctgggtccggaacggattttaccctgaagataagtcgagttgaagcggaagacctgggcatatactattgcttccaggcgtcccacgtcccctatacatttgggggtgggactaaactggaaattaaa

SEQ ID NO: 89-90 - skipped (2 entries)

SEQ ID NO: 91 - protein, 45 aa, Synthetic construct
TTTPAPRPPTPAPTIASQPLSLRPEACRPAAGGAVHTRGLDFACD

SEQ ID NO: 92 - DNA, 135 bp, Synthetic construct
  source 1..135: mol_type = other DNA
acgacgacaccagccccaagacccccaactcccgctcctaccatagcttcccaacccctgtcactgaggccagaggcatgcaggcccgctgcgggtggcgcggtacatacgcggggactggactttgcatgtgac

SEQ ID NO: 93 - protein, 37 aa, Synthetic construct
IYIWAPLAGTCGVLLLSLVITLYCNHRNRRRVCKCPR

SEQ ID NO: 94 - DNA, 111 bp, Synthetic construct
  source 1..111: mol_type = other DNA
atttacatatgggcgccactggcgggaacatgtggagttttgttgcttagcctggtcataacactgtattgcaatcatcgcaacagacgccgcgtctgcaagtgtcccagg

SEQ ID NO: 95-96 - skipped (2 entries)

SEQ ID NO: 97 - protein, 41 aa, Synthetic construct
RSKRSRLLHSDYMNMTPRRPGPTRKHYQPYAPPRDFAAYRS

SEQ ID NO: 98 - DNA, 123 bp, Synthetic construct
  source 1..123: mol_type = other DNA
agatctaagagaagtagacttcttcacagtgattatatgaatatgacgcctcgaagacccggcccgacacgcaaacactatcagccgtatgccccccctcgggattttgctgcctaccgcagc

SEQ ID NO: 99 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 336 bp, Synthetic construct
  source 1..336: mol_type = other DNA
cgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctagg

SEQ ID NO: 101 - protein, 521 aa, Synthetic construct
  VARIANT 201: note = x is i or v
MALPVTALLLPLALLLHAARPGSELPTQGTFSNVSTNVSSPEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSAGGGGSGGGGSGGGGSDVLLTQTPLSLPVSLGDQASISCRSSQNIXHSDGDTYLDWFLQKPGQSPNLLIYKVSNRFSGVPDRFSGSGSGTDFTLKISRVEADDLGVYYCFQASHVPYTFGGGTKLEVKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1563 bp, Synthetic construct
  source 1..1563: mol_type = other DNA
  misc_difference 601: note = r is a or g
  misc_difference 603: note = w is a or t
atggctcttccggtaaccgctctgctgcttcctttggctctgcttcttcacgcggctaggccagggagcgaattgcctacgcaggggactttttcaaatgtaagcacgaatgtgagctctccggaagtcaaattggtggagagtggcggtgggctcgtaaaaccaggaggcagcttgaaagtaagttgcacagcgtctgggttcactttcagccgatacgctatgagttgggtgcggcaaacgcccgaaagacgattggagtgggtagctagtattacttttgggggttccgcgtattacttggatagcgttaaagggagattcaccatatcacgagataatgcccaaaacatactctacctccagatgaactcattggtgtctgaagatactgcaatttacaactgcgccaggcaccagccatggtttgactattggggtcaaggtacgttggtcacagtctcagccggcggtggcggatctggagggggaggcagtggaggaggtggttcagatgttctcctgacgcaaactcctttgtcattgccggtaagtctgggcgaccaagcgagcatttcctgtcgcagcagccagaacatartwcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaacgtacgacgacaccagccccaagacccccaactcccgctcctaccatagcttcccaacccctgtcactgaggccagaggcatgcaggcccgctgcgggtggcgcggtacatacgcggggactggactttgcatgtgacatttacatatgggcgccactggcgggaacatgtggagttttgttgcttagcctggtcataacactgtattgcaatcatcgcaacagacgccgcgtctgcaagtgtcccagggtcgacagatctaagagaagtagacttcttcacagtgattatatgaatatgacgcctcgaagacccggcccgacacgcaaacactatcagccgtatgccccccctcgggattttgctgcctaccgcagccgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctagg

SEQ ID NO: 103 - protein, 21 aa, Synthetic construct
MALPVTALLLPLALLLHAARP

SEQ ID NO: 104 - DNA, 63 bp, Synthetic construct; identical to SEQ ID NO: 76
  source 1..63: mol_type = other DNA

SEQ ID NO: 105-106 - skipped (2 entries)

SEQ ID NO: 107 - protein, 16 aa, Synthetic construct
ELPTQGTFSNVSTNVS

SEQ ID NO: 108 - DNA, 48 bp, Synthetic construct; identical to SEQ ID NO: 80
  source 1..48: mol_type = other DNA

SEQ ID NO: 109-110 - skipped (2 entries)

SEQ ID NO: 111 - protein, 115 aa, Synthetic construct; identical to SEQ ID NO: 38

SEQ ID NO: 112 - DNA, 345 bp, Synthetic construct
  source 1..345: mol_type = other DNA
gaagtcaaattggtggagagtggcggtgggctcgtaaaaccaggaggcagcttgaaagtaagttgcacagcgtctgggttcactttcagccgatacgctatgagttgggtgcggcaaacgcccgaaagacgattggagtgggtagctagtattacttttgggggttccgcgtattacttggatagcgttaaagggagattcaccatatcacgagataatgcccaaaacatactctacctccagatgaactcattggtgtctgaagatactgcaatttacaactgcgccaggcaccagccatggtttgactattggggtcaaggtacgttggtcacagtctcagcc

SEQ ID NO: 113 - protein, 15 aa, Synthetic construct
GGGGSGGGGSGGGGS

SEQ ID NO: 114 - DNA, 45 bp, Synthetic construct
  source 1..45: mol_type = other DNA
ggcggtggcggatctggagggggaggcagtggaggaggtggttca

SEQ ID NO: 115 - protein, 112 aa, Synthetic construct; identical to SEQ ID NO: 47
  VARIANT 30: note = x is i or v

SEQ ID NO: 116 - DNA, 336 bp, Synthetic construct
  source 1..336: mol_type = other DNA
  misc_difference 90: note = w is a or t
  misc_difference 88: note = r is a or g
gatgttctcctgacgcaaactcctttgtcattgccggtaagtctgggcgaccaagcgagcatttcctgtcgcagcagccagaacatartwcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaa

SEQ ID NO: 117-118 - skipped (2 entries)

SEQ ID NO: 119 - protein, 45 aa, Synthetic construct; identical to SEQ ID NO: 91

SEQ ID NO: 120 - DNA, 135 bp, Synthetic construct; identical to SEQ ID NO: 92
  source 1..135: mol_type = other DNA

SEQ ID NO: 121 - protein, 37 aa, Synthetic construct; identical to SEQ ID NO: 93

SEQ ID NO: 122 - DNA, 111 bp, Synthetic construct; identical to SEQ ID NO: 94
  source 1..111: mol_type = other DNA

SEQ ID NO: 123-124 - skipped (2 entries)

SEQ ID NO: 125 - protein, 41 aa, Synthetic construct; identical to SEQ ID NO: 97

SEQ ID NO: 126 - DNA, 123 bp, Synthetic construct; identical to SEQ ID NO: 98
  source 1..123: mol_type = other DNA

SEQ ID NO: 127 - protein, 112 aa, Synthetic construct; identical to SEQ ID NO: 99

SEQ ID NO: 128 - DNA, 336 bp, Synthetic construct; identical to SEQ ID NO: 100
  source 1..336: mol_type = other DNA

SEQ ID NO: 129 - protein, 630 aa, Synthetic construct
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 130 - protein, 622 aa, Synthetic construct
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 131 - protein, 8 aa, Synthetic construct
PQAPRRPL

SEQ ID NO: 132 - protein, 12 aa, Synthetic construct
RPLPQVATLIDR

SEQ ID NO: 133 - protein, 14 aa, Synthetic construct
GHEMSPQVATLIDR

SEQ ID NO: 134 - DNA, 69 bp, Synthetic construct
  source 1..69: mol_type = other DNA
atgcagatcccccaggccccctggcccgtggtgtgggccgtgctgcagctgggctggaggcccggctgg

SEQ ID NO: 135 - protein, 23 aa, Synthetic construct
MQIPQAPWPVVWAVLQLGWRPGW

SEQ ID NO: 136 - DNA, 441 bp, Synthetic construct
  source 1..441: mol_type = other DNA
ttcctggacagccccgacaggccctggaacccccccaccttcagccccgccctgctggtggtgaccgagggcgacaacgccaccttcacctgcagcttcagcaacaccagcgagagcttcgtgctgaactggtacaggatgagccccagcaaccagaccgacaagctggccgccttccccgaggacaggagccagcccggccaggactgcaggttcagggtgacccagctgcccaacggcagggacttccacatgagcgtggtgagggccaggaggaacgacagcggcacctacctgtgcggcgccatcagcctggcccccaaggcccagatcaaggagagcctgagggccgagctgagggtgaccgagaggagggccgaggtgcccaccgcccaccccagccccagccccaggcccgccggccagttccagaccctggtg

SEQ ID NO: 137 - protein, 147 aa, Synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 138 - DNA, 36 bp, Synthetic construct
  source 1..36: mol_type = other DNA
tgccccagccccctgttccccggccccagcaagccc

SEQ ID NO: 139 - protein, 12 aa, Synthetic construct
CPSPLFPGPSKP

SEQ ID NO: 140 - DNA, 81 bp, Synthetic construct
  source 1..81: mol_type = other DNA
ttctgggtgctggtggtggtgggcggcgtgctggcctgctacagcctgctggtgaccgtggccttcatcatcttctgggtg

SEQ ID NO: 141 - protein, 27 aa, Synthetic construct
FWVLVVVGGVLACYSLLVTVAFIIFWV

SEQ ID NO: 142 - DNA, 72 bp, Synthetic construct
  source 1..72: mol_type = other DNA
ctgtgcgccaggcccaggaggagccccgcccaggaggacggcaaggtgtacatcaacatgcccggcaggggc

SEQ ID NO: 143 - protein, 24 aa, Synthetic construct
LCARPRRSPAQEDGKVYINMPGRG

SEQ ID NO: 144 - DNA, 339 bp, Synthetic construct
  source 1..339: mol_type = other DNA
agggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 145 - protein, 113 aa, Synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 146 - DNA, 1038 bp, Synthetic construct
  source 1..1038: mol_type = other DNA
atgcagatcccccaggccccctggcccgtggtgtgggccgtgctgcagctgggctggaggcccggctggttcctggacagccccgacaggccctggaacccccccaccttcagccccgccctgctggtggtgaccgagggcgacaacgccaccttcacctgcagcttcagcaacaccagcgagagcttcgtgctgaactggtacaggatgagccccagcaaccagaccgacaagctggccgccttccccgaggacaggagccagcccggccaggactgcaggttcagggtgacccagctgcccaacggcagggacttccacatgagcgtggtgagggccaggaggaacgacagcggcacctacctgtgcggcgccatcagcctggcccccaaggcccagatcaaggagagcctgagggccgagctgagggtgaccgagaggagggccgaggtgcccaccgcccaccccagccccagccccaggcccgccggccagttccagaccctggtgtgccccagccccctgttccccggccccagcaagcccttctgggtgctggtggtggtgggcggcgtgctggcctgctacagcctgctggtgaccgtggccttcatcatcttctgggtgctgtgcgccaggcccaggaggagccccgcccaggaggacggcaaggtgtacatcaacatgcccggcaggggcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 147 - protein, 346 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 148 - DNA, 63 bp, Synthetic construct; identical to SEQ ID NO: 76
  source 1..63: mol_type = other DNA

SEQ ID NO: 149 - protein, 21 aa, Synthetic construct
MALPVTALLLPLALLLHAARP

SEQ ID NO: 150-151 - skipped (2 entries)

SEQ ID NO: 152 - DNA, 48 bp, Synthetic construct; identical to SEQ ID NO: 80
  source 1..48: mol_type = other DNA

SEQ ID NO: 153 - protein, 16 aa, Synthetic construct
ELPTQGTFSNVSTNVS

SEQ ID NO: 154 - DNA, 21 bp, Synthetic construct
  source 1..21: mol_type = other DNA
tctccgggatccccaggatgg

SEQ ID NO: 155 - protein, 7 aa, Synthetic construct
SPGSPGW

SEQ ID NO: 156 - DNA, 441 bp, Synthetic construct
  source 1..441: mol_type = other DNA
ttcctggacagcccagatcggccctggaatccccctaccttttcccctgccctgctggtggtgacagagggcgacaacgccaccttcacatgcagcttttccaacacctctgagagcttcgtgctgaattggtacagaatgtccccatctaaccagacagataagctggccgcatttccagaggacaggtcccagcctggacaggattgccgcttccgggtgacccagctgcccaatggcagagactttcacatgagcgtggtgagggcccggagaaacgattccggcacatacctgtgcggagccatctctctggccccaaaggcacagatcaaggagagcctgagggcagagctgagggtgaccgagaggagggcagaggtgcctacagcacacccaagcccttccccaagaccagcaggacagttccagaccctggtg

SEQ ID NO: 157 - protein, 147 aa, Synthetic construct; identical to SEQ ID NO: 137

SEQ ID NO: 158 - DNA, 36 bp, Synthetic construct
  source 1..36: mol_type = other DNA
tgcccttccccactgttcccaggaccttctaagccc

SEQ ID NO: 159 - protein, 147 aa, Synthetic construct; identical to SEQ ID NO: 137

SEQ ID NO: 160 - DNA, 81 bp, Synthetic construct
  source 1..81: mol_type = other DNA
ttttgggtgctggtggtggtgggaggcgtgctggcctgctactctctgctggtgacagtggccttcatcatcttttgggtg

SEQ ID NO: 161 - protein, 27 aa, Synthetic construct
FWVLVVVGGVLACYSLLVTVAFIIFWV

SEQ ID NO: 162 - DNA, 72 bp, Synthetic construct
  source 1..72: mol_type = other DNA
ctgtgcgcccgccctcggagaagcccagcacaggaggacggcaaggtgtatatcaatatgccaggcagaggc

SEQ ID NO: 163 - protein, 24 aa, Synthetic construct
LCARPRRSPAQEDGKVYINMPGRG

SEQ ID NO: 164 - DNA, 339 bp, Synthetic construct
  source 1..339: mol_type = other DNA
agggtgaagttttctcggagcgccgatgcaccagcataccagcagggacagaaccagctgtataacgagctgaatctgggcaggcgcgaggagtacgacgtgctggataagcggagaggcagagacccagagatgggaggcaagccacagaggaggaagaaccctcaggagggcctgtacaatgagctgcagaaggacaagatggccgaggcctattccgagatcggcatgaagggagagcggagaaggggcaagggacacgatggcctgtaccagggcctgtctaccgccacaaaggacacctatgatgccctgcacatgcaggccctgccacccagg

SEQ ID NO: 165 - protein, 113 aa, Synthetic construct; identical to SEQ ID NO: 145

SEQ ID NO: 166 - DNA, 1107 bp, Synthetic construct
  source 1..1107: mol_type = other DNA
atggctcttccggtaaccgctctgctgcttcctttggctctgcttcttcacgcggctaggccagggagcgaattgcctacgcaggggactttttcaaatgtaagcacgaatgtgagctctccgggatccccaggatggttcctggacagcccagatcggccctggaatccccctaccttttcccctgccctgctggtggtgacagagggcgacaacgccaccttcacatgcagcttttccaacacctctgagagcttcgtgctgaattggtacagaatgtccccatctaaccagacagataagctggccgcatttccagaggacaggtcccagcctggacaggattgccgcttccgggtgacccagctgcccaatggcagagactttcacatgagcgtggtgagggcccggagaaacgattccggcacatacctgtgcggagccatctctctggccccaaaggcacagatcaaggagagcctgagggcagagctgagggtgaccgagaggagggcagaggtgcctacagcacacccaagcccttccccaagaccagcaggacagttccagaccctggtgtgcccttccccactgttcccaggaccttctaagcccttttgggtgctggtggtggtgggaggcgtgctggcctgctactctctgctggtgacagtggccttcatcatcttttgggtgctgtgcgcccgccctcggagaagcccagcacaggaggacggcaaggtgtatatcaatatgccaggcagaggcagggtgaagttttctcggagcgccgatgcaccagcataccagcagggacagaaccagctgtataacgagctgaatctgggcaggcgcgaggagtacgacgtgctggataagcggagaggcagagacccagagatgggaggcaagccacagaggaggaagaaccctcaggagggcctgtacaatgagctgcagaaggacaagatggccgaggcctattccgagatcggcatgaagggagagcggagaaggggcaagggacacgatggcctgtaccagggcctgtctaccgccacaaaggacacctatgatgccctgcacatgcaggccctgccacccagg

SEQ ID NO: 167 - protein, 369 aa, Synthetic construct
MALPVTALLLPLALLLHAARPGSELPTQGTFSNVSTNVSSPGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 168 - protein, 521 aa, Synthetic construct
MALPVTALLLPLALLLHAARPGSELPTQGTFSNVSTNVSSPEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSAGGGGSGGGGSGGGGSDVLLTQTPLSLPVSLGDQASISCRSSQNIVHSDGDTYLDWFLQKPGQSPNLLIYKVSNRFSGVPDRFSGSGSGTDFTLKISRVEADDLGVYYCFQASHVPYTFGGGTKLEVKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - DNA, 1563 bp, Synthetic construct
  source 1..1563: mol_type = other DNA
atggctcttccggtaaccgctctgctgcttcctttggctctgcttcttcacgcggctaggccagggagcgaattgcctacgcaggggactttttcaaatgtaagcacgaatgtgagctctccggaagtcaaattggtggagagtggcggtgggctcgtaaaaccaggaggcagcttgaaagtaagttgcacagcgtctgggttcactttcagccgatacgctatgagttgggtgcggcaaacgcccgaaagacgattggagtgggtagctagtattacttttgggggttccgcgtattacttggatagcgttaaagggagattcaccatatcacgagataatgcccaaaacatactctacctccagatgaactcattggtgtctgaagatactgcaatttacaactgcgccaggcaccagccatggtttgactattggggtcaaggtacgttggtcacagtctcagccggcggtggcggatctggagggggaggcagtggaggaggtggttcagatgttctcctgacgcaaactcctttgtcattgccggtaagtctgggcgaccaagcgagcatttcctgtcgcagcagccagaacatagttcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaacgtacgacgacaccagccccaagacccccaactcccgctcctaccatagcttcccaacccctgtcactgaggccagaggcatgcaggcccgctgcgggtggcgcggtacatacgcggggactggactttgcatgtgacatttacatatgggcgccactggcgggaacatgtggagttttgttgcttagcctggtcataacactgtattgcaatcatcgcaacagacgccgcgtctgcaagtgtcccagggtcgacagatctaagagaagtagacttcttcacagtgattatatgaatatgacgcctcgaagacccggcccgacacgcaaacactatcagccgtatgccccccctcgggattttgctgcctaccgcagccgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctagg

SEQ ID NO: 170 - protein, 21 aa, Synthetic construct
MALPVTALLLPLALLLHAARP

SEQ ID NO: 171 - DNA, 63 bp, Synthetic construct; identical to SEQ ID NO: 76
  source 1..63: mol_type = other DNA

SEQ ID NO: 172-173 - skipped (2 entries)

SEQ ID NO: 174 - protein, 16 aa, Synthetic construct
ELPTQGTFSNVSTNVS

SEQ ID NO: 175 - DNA, 48 bp, Synthetic construct; identical to SEQ ID NO: 80
  source 1..48: mol_type = other DNA

SEQ ID NO: 176-177 - skipped (2 entries)

SEQ ID NO: 178 - protein, 115 aa, Synthetic construct; identical to SEQ ID NO: 38

SEQ ID NO: 179 - DNA, 345 bp, Synthetic construct; identical to SEQ ID NO: 112
  source 1..345: mol_type = other DNA

SEQ ID NO: 180 - protein, 15 aa, Synthetic construct
GGGGSGGGGSGGGGS

SEQ ID NO: 181 - DNA, 45 bp, Synthetic construct; identical to SEQ ID NO: 114
  source 1..45: mol_type = other DNA

SEQ ID NO: 182 - protein, 112 aa, Synthetic construct
DVLLTQTPLSLPVSLGDQASISCRSSQNIVHSDGDTYLDWFLQKPGQSPNLLIYKVSNRFSGVPDRFSGSGSGTDFTLKISRVEADDLGVYYCFQASHVPYTFGGGTKLEVK

SEQ ID NO: 183 - DNA, 336 bp, Synthetic construct
  source 1..336: mol_type = other DNA
gatgttctcctgacgcaaactcctttgtcattgccggtaagtctgggcgaccaagcgagcatttcctgtcgcagcagccagaacatagttcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaa

SEQ ID NO: 184-185 - skipped (2 entries)

SEQ ID NO: 186 - protein, 45 aa, Synthetic construct; identical to SEQ ID NO: 91

SEQ ID NO: 187 - DNA, 135 bp, Synthetic construct; identical to SEQ ID NO: 92
  source 1..135: mol_type = other DNA

SEQ ID NO: 188 - protein, 37 aa, Synthetic construct; identical to SEQ ID NO: 93

SEQ ID NO: 189 - DNA, 111 bp, Synthetic construct; identical to SEQ ID NO: 94
  source 1..111: mol_type = other DNA

SEQ ID NO: 190-191 - skipped (2 entries)

SEQ ID NO: 192 - protein, 41 aa, Synthetic construct; identical to SEQ ID NO: 97

SEQ ID NO: 193 - DNA, 123 bp, Synthetic construct; identical to SEQ ID NO: 98
  source 1..123: mol_type = other DNA

SEQ ID NO: 194 - protein, 112 aa, Synthetic construct; identical to SEQ ID NO: 99

SEQ ID NO: 195 - DNA, 336 bp, Synthetic construct; identical to SEQ ID NO: 100
  source 1..336: mol_type = other DNA

SEQ ID NO: 196 - protein, 500 aa, Synthetic construct
GSELPTQGTFSNVSTNVSSPEVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSAGGGGSGGGGSGGGGSDVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 500 aa, Synthetic construct
  VARIANT 180: note = x is i or v
GSELPTQGTFSNVSTNVSSPEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSAGGGGSGGGGSGGGGSDVLLTQTPLSLPVSLGDQASISCRSSQNIXHSDGDTYLDWFLQKPGQSPNLLIYKVSNRFSGVPDRFSGSGSGTDFTLKISRVEADDLGVYYCFQASHVPYTFGGGTKLEVKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 500 aa, Synthetic construct
GSELPTQGTFSNVSTNVSSPEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSAGGGGSGGGGSGGGGSDVLLTQTPLSLPVSLGDQASISCRSSQNIVHSDGDTYLDWFLQKPGQSPNLLIYKVSNRFSGVPDRFSGSGSGTDFTLKISRVEADDLGVYYCFQASHVPYTFGGGTKLEVKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 323 aa, Synthetic construct
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 200 - protein, 348 aa, Synthetic construct
GSELPTQGTFSNVSTNVSSPGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 201 - protein, 50 aa, Synthetic construct
EDIRKWNVTSLETLKALLEVNKGHEMSPQVATLIDRFVKGRGQLDKDTLD

SEQ ID NO: 202 - protein, 50 aa, Synthetic construct; identical to SEQ ID NO: 201

SEQ ID NO: 203 - protein, 50 aa, Synthetic construct; identical to SEQ ID NO: 201

SEQ ID NO: 204 - protein, 50 aa, Synthetic construct; identical to SEQ ID NO: 201

SEQ ID NO: 205 - protein, 50 aa, Synthetic construct; identical to SEQ ID NO: 201

SEQ ID NO: 206 - protein, 50 aa, Synthetic construct; identical to SEQ ID NO: 201

SEQ ID NO: 207 - protein, 58 aa, Synthetic construct
EDIRKWNVTSLETLKALLEVNKGHEMSPQAPRRPLPQVATLIDRFVKGRGQLDKDTLD

SEQ ID NO: 208-216 - skipped (9 entries)

SEQ ID NO: 217 - DNA, 7950 bp, synthetic construct
  source 1..7950: mol_type = other DNA
tgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacgacgccgcccacgtgaaggctgccgaccccgggggtggaccatcctctagactgccagcggccgctcgagccaccatggctcttccggtaaccgctctgctgcttcctttggctctgcttcttcacgcggctaggccagggagcgaattgcctacgcaggggactttttcaaatgtaagcacgaatgtgagctctccggaagtgaaattggtagagtctggtgggggactggttaaacctggaggcagtctgaagctctcctgcgaagccagcggctttacgttcagtcggtacgctatgtcctgggtgcgacacacaccagaaaagcgactcgcttgggtagcgagcataacgtttgggggcgatccttattacccggatggtttgaaaggtcgcttcactatcagtcgagataatacgcgaaacattctttatcttcaaatgaatagcctgcgctctgaagacaccgcgatgtattattgtgttaggcatgagtcttggtttgcatactggggtcagggtactcttgtcactgtatctgccggaggtgggggtagcggcggcggaggtagtgggggaggcggcagcgacgtgttgatgactcaaactccgcttagcttgccagtgtctcttggagaacaggtctccatatcatgtcgatccagtcaaactatcgttcataccgatgggaatatctatctcgaatggtatctgcaaaatccagggcagagtccccggcttctgatatataagattagcaatagatttagtggtgttcccgacaggttttcaggttctgggtccggaacggattttaccctgaagataagtcgagttgaagcggaagacctgggcatatactattgcttccaggcgtcccacgtcccctatacatttgggggtgggactaaactggaaattaaacgtacgacgacaccagccccaagacccccaactcccgctcctaccatagcttcccaacccctgtcactgaggccagaggcatgcaggcccgctgcgggtggcgcggtacatacgcggggactggactttgcatgtgacatttacatatgggcgccactggcgggaacatgtggagttttgttgcttagcctggtcataacactgtattgcaatcatcgcaacagacgccgcgtctgcaagtgtcccagggtcgacagatctaagagaagtagacttcttcacagtgattatatgaatatgacgcctcgaagacccggcccgacacgcaaacactatcagccgtatgccccccctcgggattttgctgcctaccgcagccgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctaggtgaacgcgtcatcatcgatccggattaggatcgatccgtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcc

SEQ ID NO: 218 - DNA, 7950 bp, synthetic construct
  source 1..7950: mol_type = other DNA
tgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacgacgccgcccacgtgaaggctgccgaccccgggggtggaccatcctctagactgccagcggccgctcgagccaccatggctcttccggtaaccgctctgctgcttcctttggctctgcttcttcacgcggctaggccagggagcgaattgcctacgcaggggactttttcaaatgtaagcacgaatgtgagctctccggaagtcaaattggtggagagtggcggtgggctcgtaaaaccaggaggcagcttgaaagtaagttgcacagcgtctgggttcactttcagccgatacgctatgagttgggtgcggcaaacgcccgaaagacgattggagtgggtagctagtattacttttgggggttccgcgtattacttggatagcgttaaagggagattcaccatatcacgagataatgcccaaaacatactctacctccagatgaactcattggtgtctgaagatactgcaatttacaactgcgccaggcaccagccatggtttgactattggggtcaaggtacgttggtcacagtctcagccggcggtggcggatctggagggggaggcagtggaggaggtggttcagatgttctcctgacgcaaactcctttgtcattgccggtaagtctgggcgaccaagcgagcatttcctgtcgcagcagccagaacatagttcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaacgtacgacgacaccagccccaagacccccaactcccgctcctaccatagcttcccaacccctgtcactgaggccagaggcatgcaggcccgctgcgggtggcgcggtacatacgcggggactggactttgcatgtgacatttacatatgggcgccactggcgggaacatgtggagttttgttgcttagcctggtcataacactgtattgcaatcatcgcaacagacgccgcgtctgcaagtgtcccagggtcgacagatctaagagaagtagacttcttcacagtgattatatgaatatgacgcctcgaagacccggcccgacacgcaaacactatcagccgtatgccccccctcgggattttgctgcctaccgcagccgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctaggtgaacgcgtcatcatcgatccggattaggatcgatccgtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcc

SEQ ID NO: 219 - DNA, 7423 bp, synthetic construct
  source 1..7423: mol_type = other DNA
tgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacgacgccgcccacgtgaaggctgccgaccccgggggtggaccatcctctagactgccagcggccgcgccaccatgcagatcccccaggccccctggcccgtggtgtgggccgtgctgcagctgggctggaggcccggctggttcctggacagccccgacaggccctggaacccccccaccttcagccccgccctgctggtggtgaccgagggcgacaacgccaccttcacctgcagcttcagcaacaccagcgagagcttcgtgctgaactggtacaggatgagccccagcaaccagaccgacaagctggccgccttccccgaggacaggagccagcccggccaggactgcaggttcagggtgacccagctgcccaacggcagggacttccacatgagcgtggtgagggccaggaggaacgacagcggcacctacctgtgcggcgccatcagcctggcccccaaggcccagatcaaggagagcctgagggccgagctgagggtgaccgagaggagggccgaggtgcccaccgcccaccccagccccagccccaggcccgccggccagttccagaccctggtgtgccccagccccctgttccccggccccagcaagcccttctgggtgctggtggtggtgggcggcgtgctggcctgctacagcctgctggtgaccgtggccttcatcatcttctgggtgctgtgcgccaggcccaggaggagccccgcccaggaggacggcaaggtgtacatcaacatgcccggcaggggcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtgataacgcgtcatcatcgatccggattaggatcgatccgtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcc

SEQ ID NO: 220 - DNA, 7496 bp, synthetic construct
  source 1..7496: mol_type = other DNA
tgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacgacgccgcccacgtgaaggctgccgaccccgggggtggaccatcctctagactgccagcggccgctcgagccaccatggctcttccggtaaccgctctgctgcttcctttggctctgcttcttcacgcggctaggccagggagcgaattgcctacgcaggggactttttcaaatgtaagcacgaatgtgagctctccgggatccccaggatggttcctggacagcccagatcggccctggaatccccctaccttttcccctgccctgctggtggtgacagagggcgacaacgccaccttcacatgcagcttttccaacacctctgagagcttcgtgctgaattggtacagaatgtccccatctaaccagacagataagctggccgcatttccagaggacaggtcccagcctggacaggattgccgcttccgggtgacccagctgcccaatggcagagactttcacatgagcgtggtgagggcccggagaaacgattccggcacatacctgtgcggagccatctctctggccccaaaggcacagatcaaggagagcctgagggcagagctgagggtgaccgagaggagggcagaggtgcctacagcacacccaagcccttccccaagaccagcaggacagttccagaccctggtgtgcccttccccactgttcccaggaccttctaagcccttttgggtgctggtggtggtgggaggcgtgctggcctgctactctctgctggtgacagtggccttcatcatcttttgggtgctgtgcgcccgccctcggagaagcccagcacaggaggacggcaaggtgtatatcaatatgccaggcagaggcagggtgaagttttctcggagcgccgatgcaccagcataccagcagggacagaaccagctgtataacgagctgaatctgggcaggcgcgaggagtacgacgtgctggataagcggagaggcagagacccagagatgggaggcaagccacagaggaggaagaaccctcaggagggcctgtacaatgagctgcagaaggacaagatggccgaggcctattccgagatcggcatgaagggagagcggagaaggggcaagggacacgatggcctgtaccagggcctgtctaccgccacaaaggacacctatgatgccctgcacatgcaggccctgccacccaggtgataacgcgtcatcatcgatccggattaggatcgatccgtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcc